(19) United States
(12) Patent Application Publication
(10) Pub. No.: US 2008/0050762 A1
(43) Pub. Date: Feb. 28, 2008

(54) METHODS AND COMPOSITIONS FOR COUPLED LUMINESCENT ASSAYS

(76) Inventors: Michael J. Corey, Bellevue, WA (US); Sumant K. Dhawan, Palo Alto, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/748,967

(22) Filed: May 15, 2007

Related U.S. Application Data
(62) Division of application No. 10/976,199, filed on Oct. 28, 2004, now abandoned.
Application No. 10/976,199 is a continuation-in-part of application No. 10/071,350, filed on Feb. 8, 2002, now Pat. No. 6,811,990.
(60) Provisional application No. 60/607,027, filed on Sep. 2, 2004.
(60) Provisional application No. 60/269,227, filed on Feb. 13, 2001.

(51) Int. Cl. C12Q 1/48

(52) U.S. Cl. 435/15

(57) ABSTRACT

The invention provides methods for measuring the amount or activity of a component in a sample using a luminescent assay comprising a luciferase capable of generating light from a high-energy molecule. In one aspect, the invention provides methods for measuring the amount of NAD+ or the activity of an enzyme or enzyme series that results in the interconversion of NDA+ and NADH. In another aspect, the invention provides methods for measuring the amount of a kinase substrate, free inorganic phosphate, and phosphatase activity. In a further aspect, the invention provides methods for measuring the amount of cAMP in a sample.

### CROSS-REFERENCES TO RELATED APPLICATIONS

This is a divisional application of U.S. application Ser. No. 10/976,199, filed Oct. 28, 2004, which is a continuation-in-part application of U.S. application Ser. No. 10/071,350, filed Feb. 8, 2002 (now U.S. Pat. No. 6,811,990), and claims the benefit of U.S. Provisional Application No. 60/607,027, filed Sep. 2, 2004. U.S. application Ser. No. 10/071,350 claims the benefit of U.S. Provisional Application No. 60/269,227, filed Feb. 13, 2001.

### FIELD OF THE INVENTION

The present invention is directed to coupled luminescent methods and compositions for use in biological assays.

### BACKGROUND OF THE INVENTION

The present invention is generally directed toward luminescent methods and compositions for measuring various biological events, such as cell death, membrane damage, cell proliferation, or enzyme activities. In these methods, something occurring as a result of enzyme activity is able to produce light, which is detected in a luminometer or other instrument capable of detecting light. The invention is more particularly directed to methods of measuring various biological events, such as cytotoxicity, membrane damage, cell proliferation, enzyme activities, or some combination of these events, by coupling the activities of enzymes, which may be supplied by the investigator, or which may have been released from dead or damaged cells, with production or consumption of high-energy molecules such as adenosine triphosphate (ATP) or nicotinamide adenine dinucleotide (reduced form) (NADH), and subsequently measuring the concentrations of these high-energy molecules by evaluation of the light produced by a light-producing molecule, such as a luciferase. The invention is also particularly directed to methods of measuring the concentrations of molecules that may be coupled by enzyme activities to production or consumption of high-energy molecules such as adenosine triphosphate (ATP) or nicotinamide adenine dinucleotide (reduced form) (NADH), and subsequently measuring the concentrations of these high-energy molecules by evaluation of the light produced by a light-producing molecule, such as a luciferase.

Cytotoxicity And Proliferation Assays: Assays for cell death and cell proliferation are very widely performed in many areas of biological and clinical research. They may be used to assess the cytotoxic effects of a drug candidate (such toxicity may be either desirable or undesirable), measure the activity of complement, measure programmed cell death (apoptosis), quantify growth-inhibitory or growth-enhancing effects, detect and characterize environmental toxins, determine the sterility or bioburden of a sample, assess drug sensitivity or resistance of a patient's tumor cells or a culture of an infectious organism, or simply determine cell number. One of the most useful and efficient applications of cell death and proliferation assays is in high-throughput screening (HTS), a collection of methods currently used by many pharmaceutical and biotechnology companies to determine the properties of large libraries of drug candidates very rapidly. However, the methods of determining cell death and proliferation currently in use all suffer from important limitations. Some of these limitations make the assays impractical for use in HTS, and also limit their utility in traditional research environments.

Assays in current use for cell death, or cytotoxicity assays, fall into several categories. One category is “release” assays, in which a substance released by dying cells is measured. Often the substance is an enzyme, such as lactate dehydrogenase (LDH) or glyceraldehyde-3-phosphate dehydrogenase (G3PDH). Traditional enzyme-release assays have exploited the fact that these enzymes create NADH, which can be observed by UJV spectroscopy at 340 nm. An alternative is to couple production of NADH to generation of a colored dye, as in the LDH-based CellTiter® assays currently available from Promega. However, these processes are slow and lack sensitivity. For example, the current product from Promega recommends seeding of 5,000-100,000 cell per well, depending on the cell type, and an incubation time with the chromogenic reagents of one hour or more. Other enzymes used in this way include phosphatases, transaminases, and argininosuccinate lyase. These enzymes are typically present in low quantities in most cells, and they do not lend themselves to simple activity assays, making the process of determining cell death cumbersome and insensitive.

Another variety of release assay involves pretreatment of the target cells with a radioactive isotope, generally ⁵¹Cr or ³H. Upon lysis, the radioactive contents are released and counted in a scintillation counter. Aside from the problems of handling and waste disposal of radioactive materials, these assays also suffer from various artifacts, and are tedious because of the pretreatment and recovery steps required. The same process can also be carried out with fluorescent dyes, such as bis-carboxyethyl-carboxyfluorescein or calcein-AM, but, again, pretreatment is required, and the dyes are spontaneously released at a significant rate by healthy cells.

Another type of release assay is the luminescent assay of ATP released from dead or damaged cells. However, as it is actually used, this is a proliferation assay, and it is discussed further below along with other proliferation assays.

Another category of cytotoxicity assay makes use of dyes which are able to invade dead cells, but not living cells. An example of such a dye is trypan blue. These assays are useful for examining individual cells, but for quantification of overall cytotoxicity they are inefficient because each cell must be counted individually, either by laborious microscopic analysis or by very expensive and time-consuming flow cytometry. Moreover, some modes of death (such as complement-mediated lysis) are not easily assessed by this method, because the dead cell remains intact for a limited period of time, after which it can no longer be counted because it has disintegrated.

Yet another category of cytotoxicity assays includes those methods directly related to apoptosis. These assays typically look for either protein markers of apoptotic processes or particular effects on DNA that are uniquely associated with apoptosis. The methods are generally slow and tedious, and thus are not suitable for high-throughput screening applications. Another method of studying apoptosis is to look at the ATP:ADP ratios in a cell, which change in a distinct way as the cell enters apoptosis. These assays may be performed by coupled luminescent methods (Bradbury et al. (2000) J. Immunol. Methods 240:79). However, while these methods are useful for qualitative definition of the mode of death, they have no advantages over the ATP-release assay in quantitative determinations of cytotoxicity or proliferation.

Proliferation assays are methods of measuring numbers of live cells. This may be better for some applications than measuring cell death or damage. For example, proliferation assays are able to reveal cytostatic, growth-inhibitory, and growth-enhancing effects which yield no readout in a cytotoxicity assay. Proliferation assays are also in common use as indirect cytotoxicity assays, but there are serious drawbacks with this approach; these are discussed below in connection with the ATP-release assay. Proliferation assays also fall into several categories. Assays of metabolic activity are in widespread use in research laboratories. The commonly used methods make use of tetrazolium salts, which are reduced in living cells to colored formazan dyes. One advantage of these methods is convenience, especially with the newer dyes (MTT and WST-1). The dye is added to the cell culture, and the absorbance of the formazan is read, typically after 0.5-12 hours. However, there are several important disadvantages. Metabolically active cells reduce the dyes at rates much greater than quiescent cells; the readout may therefore be a poor reflection of the cell number. Moreover, the readout is not an instantaneous “snapshot” of the quantity of live cells when the measurement is taken, but rather a complex integral of metabolic activity over the preceding time interval, whose mathematical relationship to the actual live cell number involves the half-life of the dye as well as variations in metabolic activity. Metabolism-based assays are not suitable for measurement of cellular cytotoxicity (for example, the activities of cytotoxic T lymphocytes), or any other assay system in which live cells other than the target cells are present, because these other cells will yield a substantial and often ill-defined background signal. Finally, various artifacts have been associated with the use of these dyes (see for example O'Brien et al, (2000) Eur. J. Biochem. 267:5421-5426; Natarajan et al. (2000) Cancer Detection and Prevention 24:405-414). Although they have not been thoroughly characterized with respect to their effects on cell metabolism, it is known that various agents, such as antioxidants, can interfere with performance of the dyes.

Another kind of proliferation assay actually measures the ability of the cells to grow. This is the colony-forming unit (CFU) assay. It is typically used with cells that grow rapidly and are capable of growth from single cells. The cells are diluted and plated on appropriate growth media, and the colonies are counted when they appear. This method is quite accurate, but is extremely tedious and quite expensive. The labor-intensive aspect of this method is exacerbated by the fact that multiple dilutions of each sample must usually be plated in order to ensure that at least one plate will yield a countable number of colonies.

Finally, cytotoxicity assays can be used as proliferation assays (and vice versa). To use a cytotoxicity assay to count live cells, one simply kills all the cells and performs the assay. (In some cases it may be necessary to wash the cells first, because the readout may depend on a molecule that may have been released into the supernatant by cells that have already died.) The most important example of this approach is the ATP-release assay, mentioned above (Crouch et al. (1993) J. Immunol. Methods 160:81). Although strictly speaking this is a cytotoxicity assay, in that ATP released by dead cells is measured, it is rarely used as a direct cytotoxicity assay, because of the very short lifetime of extracellular ATP. Instead, the cells are killed with a lytic agent before the ATP is measured by the luciferase reaction. Thus even though the assay is basically a cytotoxicity assay, if it is to be used to measure cytotoxicity, it is an indirect method, like the other proliferation assays. The ATP-release assay has a number of advantages not enjoyed by many other proliferation assays. It is more sensitive, with a limit of detection of 10-100 cells. It is much faster, with completion of the lysis and assay steps in as little as 3 minutes. Because of the sensitivity, relatively low volumes and small numbers of cells are required. It is really the only assay currently on the market that is sufficiently rapid and sensitive for use in HTS. However, important disadvantages should be noted. The ATP content of cells is subject to strong metabolic fluctuations, which will cause artifacts. Moreover, the assay can be performed only a single time, immediately after cell lysis; if that opportunity is somehow missed, the experiment must be repeated. Finally, in cytotoxicity mode, the assay suffers from very important drawbacks that are common to all proliferation assays used in this mode. The initial seeding of the wells or reaction vessels with cells must be very accurate, because the cytotoxicity readout depends on differences (which may be small) between numbers of surviving cells, and any scatter in the initial seeding contributes substantially to the noise in the results. This leads to the second problem, which is that a direct readout is almost always preferable to a signal that depends on subtracting two large numbers, as the user must do to use a proliferation assay to measure cytotoxicity. Another very important difficulty is a time-consuming problem with this approach which does not involve the actual assay step. Typically the user adds a potentially toxic compound or agent, waits for death or damage to occur, and then measures the result. The length of time the user must wait depends on the method. If the user is measuring cell death directly, then it can be measured as soon as it occurs, perhaps within minutes. However, if the user is measuring live cells in order to derive the cytotoxicity signal, then the user must wait much longer, until the cytotoxic effect has had sufficient time to cause a detectable difference between the test sample and the control. Furthermore, the required time interval is not known in advance, and if the experiment is stopped too soon, it must be repeated (or abandoned, since the user will not know whether a result showing no difference between test and control is due to the lack of an effect or insufficient time to show an effect). Thus in an HTS mode, where minutes are critical, there is an intervening step in this process requiring an interval of time which may be anywhere from 10 minutes to several days, and which cannot be predicted in advance. This is a serious drawback to the use of any proliferation assay for cytotoxicity work, including the ATP-release assay.

Another type of viability assay, also luminescent, is represented by “CytoLite,” a trade name for a mitochondrion-based viability assay (Woods and Clements (2001) Nature Labscene UK March, 2001, 38-39). This method is homogeneous, but requires a 15-minute incubation, and a further 10-minute “dark-adjustment” period before the luminance read; it is therefore too slow for high-efficiency HTS. It is also a viability assay and is subject to all of the drawbacks mentioned above as inherent to viability and proliferation assays.

A cytotoxicity assay based on release of alkaline phosphatase from target cells of killer lymphocytes was described in 1994 (Kasatori et al. (1994) Rinsho Byori 42:1050-1054). This assay method is not suitable for use with other types of cells in general, since most cells do not express alkaline phosphatase in sufficient quantity. Moreover, it involves the use of a substrate whose general effects on cells have not been characterized. It is not a homogeneous or high-throughput assay.

A luminescent cytotoxicity assay described in a 1997 report is based on stable transfection of target cell lines of interest with luciferase or B-galactosidase (Schafer et al. (1997) J. Immunol. Meth. 204:89-98. In terms of sensitivity, this assay represents an advance over conventional release assays; however, the disadvantages of this approach are serious. First, stable transfection itself is a labor-intensive and expensive procedure; yet this must be done for every target cell line of interest if the method of Schafer et al, is to be used. Stable transfection does not always work, and, if it does, may alter the metabolic characteristics of the target cell and thereby severely complicate interpretation of the results of the experiment. The method may not be applicable to cell types outside of these that may be transfected in this manner: expression systems would be different, and the enzymes might be produced in insufficient quantities, in inactive form, or not at all. Moreover, the assay is not homogeneous. Instead the cell culture supernatant must be separated from remaining live cells prior to running the assay. This in itself is a very serious drawback in the high-throughput screening environment, since it adds a complex step to the procedure. Finally, according to the authors, luciferase had a half-life of approximately 30 minutes under the conditions used, and this was found to be inadequate for quantification of cell death in prolonged assays.

Again in 1997, a coupled luminescent method was published (Corey et al. (1997) J. Immunol. Meth. 207:43). This method addressed several of the problems of all of the above methods. This was a release assay, but with important differences from other release assays. G3PDH activity was measured by coupling its cognate glycolytic reaction to the following reaction in glycolysis, which is carried out by phosphoglycerokinase (PGK). The PGK reaction produced ATP, which was then measured by luciferase, which was provided in a separate cocktail, yielding a luminance signal. The limit of detection was <0.1 cell, which was superior to the sensitivity of any other available assay and adequate for almost any application. The assay was relatively fast (˜12-15 minutes). Since it provided a direct readout of cytotoxicity, it suffered from none of the disadvantages of proliferation assays used in cytotoxicity mode. The luminance signal continued to increase with time, a feature which allowed the user to decide when an acceptable signal had been achieved “on the fly.” Nevertheless, the GPL assay had its own disadvantages which prevented it from being commercially viable. It was cumbersome to execute, in that it involved four transfer steps (cocktail to reaction vessel, sample to reaction vessel, luciferase to luminance vessel, aliquot of reaction to luciferase) and two incubations prior to the actual read. Moreover, because the assay cocktail was not compatible with live cells, tests involving bacteria, erythrocytes, or other non-adherent cells or microbes were still more tedious, because the live cells had to be separated from the supernatant by centrifugation prior to the assay. Finally, like all the methods described above, the assay could be used in cytotoxicity mode or in proliferation mode (the latter by killing all the cells prior to the readout), but not both, with a single sample. These features contributed to the unsuitability of the GPL assay for use in high-throughput screening, especially the necessity of several transfers and the separation of the cells from the supernatant. It was also of limited utility for research use because of its complexity of operation.

As mentioned above, an important disadvantage shared by most cytotoxicity and proliferation assays currently available is that they do not permit measurement of both cytotoxicity and proliferation in a single sample. Release assays, such as the GPL assay, permit quantification of cell rupture or damage, but do not reveal the presence or amount of live cells present. On the other hand, proliferation assays, such as the MTT and ATP-release assays, allow quantification of live cells, in either a non-destructive (MTT) or destructive (ATP-release) mode, but yield no direct information about the degree of cell death that may have occurred. Ideally, the worker would prefer to obtain these two independent pieces of information from the same sample.

In summary, the cytotoxicity and proliferation assays currently available are far from ideal. The traditional release assays suffer from poor sensitivity and speed, Metabolism-based assays are slow, inaccurate with respect to actual cell number, and subject to serious artifacts. CFU assays are too slow and tedious for routine use.

ATP-release assays are destructive, one-time assays of moderate sensitivity, and they have numerous important drawbacks as cytotoxicity assays. Although the published coupled luminescent assay (CGPL) is superior to the other cytotoxicity and proliferation assays in many ways, it nevertheless is cumbersome and impractical for use in high-throughput screening or research environments because of the processing, numerous transfer steps, and lack of a dual cytotoxicity/proliferation mode.

Phosphatase Assays: Today's drug-discovery environment involves high-throughput screening of inhibitors or other modulators of enzyme activity. Among the enzymes of great interest are phosphatases, which participate in many vital signaling and metabolic pathways. However, assay methods in current use for phosphatases are burdened with a number of drawbacks, including poor throughput or sensitivity, the use of radioactivity, and difficulty of interpretation due to the use of unnatural substrates and/or reaction conditions. Poor throughput and/or sensitivity are often due to the nature of the assay; for example, assays utilizing antibodies against phosphorylated target molecules generally require extended incubations, assays making use of electrophoretic separations are too slow to allow the throughput desired, and assays using radioactivity are inherently inconvenient and also suffer from poor throughput. In particular, fluorescence polarization (FP) assays are currently under consideration for high-throughput procedures in some cases. However, these assays, which generally make use of antibodies or other ligands directed against phosphorylated target molecules for detection of phosphatase activity, generally require long incubation times for ligand-target association that significantly reduce the value of these assays in high-throughput screening. These assays also typically involve multiple additions of antibodies or other ligands, and/or wash steps, as well as the design, synthesis, and subsequent ongoing cost of fluorophore-containing biomolecules or synthetic compounds. There is also the possibility that a molecule under study as a modulator of phosphatase activity will give a false signal by binding the fluorophore itself, by otherwise quenching or enhancing its fluorescence, or by blocking the target site on the phosphorylated protein. Finally, many FP assays, and other assays which rely on detection of a phosphorylated target molecule, suffer from an additional disadvantage in that the phosphatase activity yields a negative signal, i.e., a decrease in the phosphorylated molecule which is the target of detection. Such a negative signal is generally considered inferior to a positive signal in enzymology. For one thing, several kinds of artifacts can give rise to a negative signal, including protease contamination or unexpected denaturation of a critical protein. Moreover, a negative signal is usually limited in its dynamic range by its very nature.

Another class of phosphatase assay strategies is based on detection of phosphate liberated by the enzymatic activity. One possibility is radiolabeling of the phosphate group, which can then be separated and counted in some manner. Although this method is still in use in research, it is extremely inconvenient, involving the expense of the label itself, the difficulty and expense of creating or' purchasing the labeled compound, a separation step, and the danger and tedium of dealing with the radioactive products. The primary non-radioactive method of detecting phosphate is the use of the malachite green reaction (Mahuren et al. (2001) Anal. Biochem. 298:241), which is quite slow and involves multiple reaction steps, making it unsuitable for high-throughput applications. Another methods of detecting phosphate, which is a coupled luminescent scheme, is useful in devices for environmental or food sampling (Karube, M. (1998) Japanese Patent Application Number 10121688), but involves multiple mixing steps and the use of immobilized enzymes with flow cells in a portable sampling device, making it unsuitable for a high-throughput screening environment. In any case this method has never been shown to be compatible with phosphatase activities. Moreover the oxidizing agents produced in the detection reaction (including hydrogen peroxide) might inactivate a large class of important phosphatases containing active-site thiol groups.

In contrast to the phosphatase assay strategies mentioned above, which can make use of either natural or general peptide/protein substrates, other strategies make use of molecules that are designed more to ease the problem of detection than as ideal substrates for the phosphatase under study. The use of these highly unnatural substrates in high-throughput screening procedures poses a different set of problems, especially problems of interpretation. In most cases the unnatural substrate has quite different kinetic parameters from the actual in vivo substrate. The corollary of this is that when inhibitors or modulators of phosphatase activity are found by such procedures, their characteristics in reactions with the actual in vivo substrate may prove to be very different, especially if competitive inhibition is involved. This is even more likely to be the case if the unnatural substrate has a substantially higher Kₘ (Michaelis constant) for the enzyme than the natural substrate, since competitive inhibitors identified in such a system may successfully compete for the weakly binding unnatural substrate, but may be ineffective against the strongly binding, natural substrate. Similarly, important inhibitors may not be identified by such a system, especially if the substrate is smaller, more labile than, or kinetically distinct from the natural substrate. For example, p-nitrophenylphosphate is a commercially important substrate for alkaline phosphatase, because it is very labile and yields a colorimetric result, but its use in inhibitor screening applications could lead to false rejection of good inhibitors. An inhibitor might be strong enough to exhibit useful inhibition of the natural reaction, but not strong enough to prevent most of this very labile ester from being hydrolyzed. Similarly, the inhibitor might block the active site in such a way that the natural reaction is prevented, but small molecules such as p-nitrophenylphosphate, phenacyl phosphate, luciferin phosphate, or 1,2 dioxetanes (see below) can still enter the active site and be hydrolyzed. This could lead to rejection of valuable “hits” in a screening situation. In short, when the reaction being studied is not the same as the natural reaction that is the desired target, there is a substantial risk that the information gathered will not be biologically useful or relevant.

A luminescent phosphatase assay has been reported that employs a 1,2 dioxetane as a substrate (Adam et al. (1996) Analyst 121:1527; Olesen et al. (2000) Methods Enzymol. 326:175). A related method employs a substrate that leads to generation of a dioxetane in situ (Catalani et al. (1999) Analytica Chimica Acta 402:99). A third method employs phenacyl phosphate as the substrate, followed by reaction with lucigenin (Sasamoto et al, (1995) Anal. Chim. Acta 306:161). These methods work only with alkaline phosphatases, and are not readily extensible to other phosphatases, since a new substrate and/or reaction series might have to be designed and synthesized for each phosphatase. In many or most cases this may be impossible or prohibitively expensive. Alkaline phosphatases typically have very different substrate specificities from the protein phosphatases that are of greatest interest in today's biology, such as protein tyrosine phosphatases and serine/threonine phosphatases. Moreover, the methods are not rapid, homogeneous assays, for example, the assay recently reported by Olesen et al. involves 3-4 transfers and at least 2 separate incubations, over a period of at least 30 minutes. This would make it most inconvenient for a high-throughput setting. Another serious drawback of these approaches, discussed above, is the use of unnatural substrates.

Another molecule that has been used as a substrate in phosphatase assays is luciferin phosphate (Mountfort et al. (1999) Toxicon 37:909; Miska and Geiger (1988) Biol. Chem. Hoppe-Seyler 369:407). The principle of the assay is that generation of free luciferin by hydrolysis of luciferin phosphate (catalyzed by the phosphatase) may lead to light production in a reaction that contains luciferase and ATP, but a limiting amount of luciferin. In the 1988 work alkaline phosphate was used, but in the 1999 work, luciferin phosphate was used in an assay of protein phosphatase 2A. In both cited references the assay was slow (30-60 minutes for the enzymatic-reaction step alone), and non-homogeneous (involving at least one transfer after initiation). While it is interesting that protein phosphatase 2A hydrolyzes this highly unnatural substrate, the rate of hydrolysis was so poor that the detection limit was more than 1000-fold worse than by fluorimetric methods (however, these fluorimetric methods also required one hour, involved multiple steps, and required highly unnatural substrates). While it is unknown whether this work can be transferred to other protein phosphatases, it is clear that such hypothetical methods, if possible, would likely be insensitive, very slow, and non-homogeneous, and would also make use of unnatural substrates, with all the disadvantages discussed above.

Detection of Cyclic AMP: Cyclic adenosine monophosphate (cAMP) is a highly important signaling molecule in many cells. Detection and/or quantification of cAMP is desirable in studies of G-protein coupled receptors, phosphodiesterase-mediated biological effects, and other systems. However, current methods of detecting and/or quantifying cAMP have important drawbacks. Traditional methods involve laborious preparations of extracts and/or radioactive tracers, with many attendant disadvantages. The “Hit-Hunter” kit offered by Applied Biosystems and DiscoverX is sensitive to concentrations of cAMP in the nanomolar range, but the assay system is extremely complicated, involving complementation of a proteolytically cleaved galactosidase enzyme by a cAMP-complexed fragment that is usually bound to a specific antibody, but is released when free cAMP is present. The numerical output of the assay reflects the fact that detection of cAMP is highly indirect in this system. According to the product literature, varying cAMP from 10⁻¹ to 10² pmol/well (the center of the claimed dynamic range), or three orders of magnitude, results in a change in signal of approximately 10-fold, and the variation pattern is highly non-linear. Thus it may be hard to draw quantitative conclusions about cAMP concentration in this system. Moreover, the assay requires at least 3.5 hours, according DiscoverX product literature.

The LANCE system from Perkin Elmer employs an anti-cAMP antibody with a cAMP derivative labeled with allophycocyanin. cAMP in the sample competes with the allophycocyanin derivative, allowing release of the derivative and modulation of the time-resolved fluorescence signal of europium in a homogeneous assay. This method is conceptually and biochemically complex, involving components that are expensive to prepare, and like most techniques involving antibody association or dissociation, it is relatively slow. Moreover, it requires the use of a time-resolved fluorescence instrument, which most research laboratories do not possess.

U.S. Pat. No. 5,891,659 (Murakami et al.) provides a method of measuring cAMP by coupled luminescence. Previous methods (e.g., Methods in Enzymology 38:62-65; 1974) required addition of ATP to a system in which ATP was cyclically regenerated. This led to a considerable background signal. However, Murakami et al. provide a method in which cAMP is converted to AMP, and ATP is then generated by a coupled enzyme system employing pyruvate orthophosphate dikinase, together with the “high-energy” substrate phosphoenol pyruvate. In this system, the ATP concentration, and therefore light production, is directly related to the initial cAMP concentration, without the need for addition of ATP. However, pyruvate orthophosphate dikinase is not commercially available, and is a complex enzyme that is difficult to handle successfully. Purification of the enzyme from natural sources is very laborious, as described in U.S. Pat. No. 5,891,659.

Detection and/or Quantification of Nicotinamide Adenine Dinucleotide (Oxidized Form) and/or Nicotinamide Adenine Dinucleotide Phosphate (Oxidized Form): Most currently used methods of detecting nicotinamide adenine dinucleotide (oxidized form) (NAD⁺) and/or nicotinamide adenine dinucleotide phosphate (oxidized form) (NADP⁺) are photometric, although certain fluorescent methods are available. NAD can be converted to NADH by the reaction of LDH with lactate or a similar redox enzyme system; the NADH is then coupled to diaphorase or N-methylphenazinium, where is reduces resazurin or a similar dye (nonfluorescent) to produce resorufin (fluorescent). This in turn generates NAD, which is cycled back into the system. This system is sensitive because of the amplification loop, in which a small amount of NAD⁺ can lead to larger quantities of fluorescent product, but it is not specific for NAD⁺. NAD⁺ in this system must be converted to NADH before it can be measured, and the system cannot distinguish between the two. A coupled luminescent enzymatic method of detection of NAD⁺ and NADP⁺ would allow harnessing of the very high specificity of enzyme reactions to give rise to a detection event only in response to NAD⁺ and NADP⁺. This process could be coupled with the activity of other enzymes to connect the process to enzymes involved with phosphate metabolism, and eventually to production of ATP and light generation by an ATP-dependent luciferase.

Detection and/or Quantification of Nitrate: Detection of the nitrate ion (NO₃—) and of salts incorporating the nitrate ion (e.g., NaNO₃) is important in many contexts. For example, nitrate is an important component of many fertilizers, and frequently appears as an undesirable contaminant in groundwater or runoff water from agricultural operations. Many streams, lakes, rivers, oceans, and other bodies of water are regularly monitored for nitrate concentration. Moreover, detection of the nitrate ion is of growing importance in medicine. For example, studies of the highly important nitric oxide synthases (NOS) often involve measurements of nitrate concentration. However, current methods of detecting nitrate have important drawbacks. Many, such as Drop-Ex-3 from Meditests/Medimpex and EDK123 from Law Enforcement Associates, yield only qualitative results. Colorimetric and mass-spectroscopy methods generally require returning the sample to a central laboratory, and therefore have excessive turn-around times. Although some personal and portable instruments exist, these are generally expensive, with limited sensitivity, and some are quite heavy (TL-200 from Timberline Instruments for ammonia detection, for example weighs 15 kg). Hach provides the OptiQuant UV Nitrate Analyzer, which is a continuous method, but is of limited sensitivity, and is very expensive. The method is based on the absorbance of nitrate at 210 nm. The best possible sensitivity is about 2 mg/L, and a single-probe instrument lists at $13,125 as of Sep. 6, 2004. The Nico2000 is much more reasonably priced, but has a limit of detection of about 0.3 parts per million, or roughly 5 μM, which is inadequate for many applications. This electrochemical instrument is highly subject to interference from chloride and bicarbonate ions. Nitrate reductase has been used to reduce nitrate to nitrite, followed by calorimetric detection via the Griess reaction; however, for detection of NOS activity, it is necessary to add NADPH, and this interferes with the Griess reaction. Cayman provides a Nitrate/Nitrite Colorimetric Assay Kit, in which the enzyme lactate dehydrogenase is provided to consume excess NADPH, but this kit involves multiple steps, and is still subject to the other disadvantages of the Griess reaction. The Griess method involves the use of dangerous chemicals and requires several steps. Molecular Probes provides fluorescent methods for detection of nitrite, but these methods are not suitable for specific detection of nitrate without at least one additional step. An ideal assay would use a relatively inexpensive, easily portable or disposable device and give a quantitative answer in a few minutes in a one-step reaction, fast enough, for example, to tip the worker off to take further measurements at the same site, in environmental monitoring applications.

Measurement of Enzymatic Activity of Lactate Dehydrogenase: Lactate dehydrogenase (LDH) is an enzyme present in all known living cells, and closely related to the energy-producing glycolytic pathway. Its presence in cell culture fluid has been very frequently used as an indicator of cell lysis, either spontaneous or intentionally produced, since the enzyme is both abundant and evidently universal. Methods of measuring LDH activity are also relatively straightforward, although they have generally been slow and unsuitable for high-throughput applications. The Promega CytoTox-ONE™ Homogeneous Membrane Integrity Assay is an advance over traditional methods involving absorbance of NADH at 340 nm, since CytoTox-ONE is homogeneous and relatively rapid and sensitive. However, faster assay methods and greater sensitivity are still desired for high-throughput applications.

Measurement of Enzymatic Activity of Acetylcholinesterase and Detection of Acetylcholinesterase Inhibitors Acetylcholinesterase (ACHE) is a critical enzyme in neurotransmission. ACHE breaks down acetylcholine, a vital neurotransmitter throughout the body, thereby deactivating it and terminating the signal. If the activity of ACHE is blocked, the body is unable to switch off signals to muscles and other organs, resulting in convulsions and death. This is the mechanism of action of the nerve gases, as well as Alzheimer's drugs such as tacrine, which inhibits ACHE more mildly, and a range of pesticides that possess specificity for the ACHE of insects or other undesirable organisms. Measurement of ACHE inhibition is also important in metabolic evaluation of drug candidates.

Unfortunately, current methods of assessing ACHE activity and detecting ACHE inhibitors have serious drawbacks. The traditional assay for ACHE activity involves colorimetric detection in a reaction employing Ellman's reagent. This assay is too slow and inadequately sensitive for use in high-throughput applications. Real-time systems can be engineered to detect specific molecules or defined sets of molecules (such as nerve gases) by mass spectrometry or other methods based on molecular weight, but these methods are very expensive, require a high degree of expertise to establish, and suffer in any case from the severe limitation that they are limited to detection of particular structures. If a substance not present in the data-base is encountered, the system has no way of detecting it reliably. A biochemical method relying on the biological activity of an acetylcholinesterase inhibitor is inherently superior to these structure-based systems, since virtually any agent exhibiting the biological effect of interest (inhibition of ACHE) is detected. An alternative is mass spectroscopy of the products of the ACHE reaction. This is offered in a so-called high-throughput mode by BioTrove, Inc. (Ozbal et al. (2004) Assay and Drug Development Technologies 2:373-382), but the equipment is very expensive, and the “high throughput” is 4-5 seconds per sample, which cannot compare with the capabilities of coupled luminescent technology-several hundred samples in the cycle time of a luminometer, which can be as little as 2 seconds.

Accordingly, there is a need in the art for assays that are practical for use in high-throughput screening. The present invention fulfills this need and further provides other related advantages.

### SUMMARY OF THE INVENTION

In one aspect, the invention provides methods for measuring the amount of oxidized form nicotinamide adenine dinucleotide (NAD⁺) in a sample. The methods comprise the steps of: (a) contacting a sample with (i) at least one enzyme that results in the generation of adenosine triphosphate (ATP) in the presence of NAD⁺ and (ii) an ATP-dependent luciferase, under conditions wherein luminance emitted from the sample depends on the amount of NAD⁺ in the sample; and (b) measuring the amount of NAD⁺ in the sample by measuring the emitted luminance. For example, these methods may be used for measuring the activity of at least one enzyme that results in the interconversion of NAD⁺ and NADH, for measuring the activity of an inhibitor of at least one enzyme that results in the interconversion of NAD⁺ and NADH in a sample, for measuring cell death or membrane damage in a sample, or for measuring the amount of a component in a sample. In some embodiments, the at least one enzyme that results in the generation of adenosine triphosphate (ATP) in the presence of NAD⁺ comprise glyceraldehyde-3-phosphate dehydrogenase and phosphoglycerokinase. Another exemplary enzyme series that results in the generation of ATP in the presence of NAD⁺ comprises isocitrate dehydrogenase, alpha-ketoglutarate dehydrogenase, succinyl-coenzyme A synthase, and a phosphate transferase, as shown in the scheme below:

1. NAD⁺+isocitrate→NADH+alpha-ketoglutarate+CO2, catalyzed by isocitrate dehydrogenase;

2. Alpha-ketoglutarate+coenzyme A+NAD⁺→NADH+CO2+succinyl-coenzyme A, catalyzed by alpha-ketoglurate dehydrogenase;

3. Succinyl-coenzyme A+GDP+Pi→GTP+coenzyme A+succinate, catalyzed by succinyl-coenzyme A synthase;

4. GTP+ADP→GDP+ATP, catalyzed by various phosphate transferases.

The methods for measuring the activity of at least one enzyme that results in the interconversion of NAD⁺ and NADH comprise the steps of: (a) contacting a sample comprising at least one enzyme that results in the interconversion of NAD⁺ and NADH with NADH or a mixture of NADH and NAD⁺, and (b) measuring the activity of the at least one enzyme that results in the interconversion of NAD⁺ and NADH by measuring the amount of NAD⁺ in the sample. The amount of NAD⁺ in the sample may be measured as described above. In some embodiments, the at least one enzyme that results in the interconversion of NAD⁺ and NADH comprises lactate dehydrogenase (LDH), and NAD⁺ is produced from NADH by LDH-catalyzed reduction of pyruvate to lactate or pyruvic acid to lactic acid. In some embodiments, the at least one enzyme that results in the interconversion of NAD⁺ and NADH comprises acetylcholinesterase and an NADH-dependent enzyme with acetate-reductase activity, and NAD⁺ is produced from NADH by the reduction of the acetate produced in the acetylcholinesterase-catalyzed reaction by the NADH-dependent enzyme with acetate-reductase activity. In some embodiments, the at least one enzyme that results in the interconversion of NAD⁺ and NADH comprises isocitrate dehydrogenase dehydrogenase, and NAD⁺ is produced from NADH by isocitrate dehydrogenase-catalyzed reduction of isocitrate to alpha-ketoglutarate.

Some embodiments provide methods for measuring the amount of one or more inhibitors of acetylcholinesterase in a sample by measuring the interconversion of NAD⁺ and NADH. The methods comprise the step of: (a) contacting a sample with acetylcholinesterase under suitable conditions for producing acetate and choline from acetylcholine; and (b) measuring the amount of one or more inhibitors of acetylcholinesterase in the sample by measuring the activity of acetylcholinesterase using the method described above for measuring the activity of at least one enzyme that results in the interconversion of NAD⁺ and NADH, in which method the at least one enzyme that results in the interconversion of NAD⁺ and NADH comprises acetylcholinesterase and an NADH-dependent enzyme with acetate-reductase activity, and NAD⁺ is produced from NADH by the reduction of the acetate produced in the acetylcholinesterase-catalyzed reaction by the NADH-dependent enzyme with acetate-reductase activity.

Some embodiments provide methods for measuring cell death or membrane damage in a sample by measuring the amount of LDH released from dead or damaged cells. The methods comprise the step of the amount of LDH released from dead or damaged cells in a sample by measuring the activity of LDH using the method described above for measuring the activity of at least one enzyme that results in the interconversion of NAD⁺ and NADH, in which method the at least one enzyme that results in the interconversion of NAD⁺ and NADH comprises LDH, and NAD⁺ is produced from NADH by the LDH-catalyzed reduction of pyruvate to lactate or pyruvic acid to lactic acid.

The methods for measuring the amount of a component in the sample comprise the steps of: (a) contacting a sample with at least one enzyme that results in the generation or consumption of NAD⁺ in the presence of a component in the sample; and (b) measuring the amount of the component in the sample by measuring the amount of NAD⁺ in the sample. The amount of NAD⁺ in the sample may be measured as described above. The component measured in the sample may be an ion or molecule that is the substrate or product of an enzyme reaction. For example, the component may be free nitrate, and the at least one enzyme that results in the generation or consumption of NAD⁺ may comprise NADH-dependent nitrate reductase.

In another aspect, the invention provides methods for measuring the amount of a kinase substrate in a sample. The methods for measuring the amount of a kinase substrate comprise the steps of: (a) contacting a sample comprising a substrate for an ATP-dependent kinase with (i) an ATP-dependent kinase in the presence of ATP to form a phosphorylated substrate and (ii) an ATP-dependent luciferase, under suitable conditions wherein luminance emitted from the sample depends on the amount of kinase substrate in the sample; and (b) measuring the amount of the substrate in the sample by measuring the emitted luminance. In some embodiments, the substrate for the ATP-dependent kinase is acetate and the ATP-dependent kinase is acetate kinase. In some embodiments, the substrate for the ATP-dependent kinase is choline and the ATP-dependent kinase is choline kinase. For example, these methods may be used for measuring the activity of acetylcholinesterase or for measuring the amount of one or more inhibitors of acetylcholinesterase.

The methods for measuring the activity of acetylcholinesterase in a sample comprise the step of measuring the activity of acetylcholinesterase in a sample by measuring the amount of acetate or choline in the sample using the method describe above for measuring the amount of a kinase substrate in a sample, wherein the acetate or choline in the sample is produced in an acetylcholinesterase-catalyzed reaction with acetylcholine. Some embodiments provide methods for measuring the amount of one or more inhibitors of acetylcholinesterase in a sample. The methods comprise the steps of: (a) contacting the sample with an acetylcholinesterase under suitable conditions for producing acetate and choline from acetylcholine; and (b) measuring the amount of one or more inhibitors of acetylcholinesterase in the sample by measuring the activity of the acetylcholinesterase. The activity of acetylcholinesterase may be measured by measuring the amount of acetate or choline in the sample using the method describe above for measuring the amount of a kinase substrate in a sample, wherein the acetate or choline in the sample is produced in an acetylcholinesterase-catalyzed reaction with acetylcholine.

In a further aspect, the invention provides methods for measuring the amount of free inorganic phosphate in a sample. The methods comprise the steps of: (a) contacting a sample with (i) at least one enzyme that results in phosphate-dependent phosphorylation of one or more substrates to form one or more phosphorylated substrates, (ii) at least one enzyme that results in generation of a high-energy molecule from the one or more phosphorylated substrates, and (iii) a luciferase capable of generating light from the high-energy molecule, under suitable conditions wherein luminance emitted from the sample depends on the concentration of free phosphate in the sample; and (b) measuring the concentration of free inorganic phosphate in the sample by measuring the emitted luminance. An exemplary enzyme that results in phosphate-dependent phosphorylation of one or more substrates to form one or more phosphorylated substrates comprises ornithine carbamoyltransferase, and an exemplary enzyme that results in the generation of a high-energy molecule from the one or more phosphorylated substrates comprises carbamoyl-phosphate synthase (glutamine-hydrolyzing), as shown in the scheme below:

1. phosphate+L-citrulline→carbamoyl phosphate+L-ornithine, catalyzed by ornithine carbamoyltransferase (“in reverse”);

2. 2 ADP+phosphate+L-glutamate+carbamoyl phosphate→2 ATP+L-glutamine+CO2+H₂O, catalyzed by carbamoyl-phosphate synthase (glutamine-hydrolyzing), EC 6.3.5.5 (“in reverse”).

For example, these methods may be used for measuring phosphatase activity in a sample. The methods for measuring phosphatase activity in a sample comprise the steps of: (a) contacting a sample with a phosphorylated substrate under suitable conditions for a phosphatase to dephosphorylate the substrate and release phosphate; and (b) measuring the phosphatase activity in the sample by measuring the amount of phosphate released by the phosphatase using the method for measuring the amount of free inorganic phosphate, as described above.

Yet another aspect of the invention provides methods for measuring the amount of cAMP in a sample. The methods comprise the steps of: (a) contacting a sample with (i) a cAMP-dependent enzyme that catalyzes a reaction yielding ATP, and (ii) an ATP-dependent luciferase, under conditions wherein luminance emitted from the sample depends on the amount of cAMP in the sample; and (b) measuring the amount of cAMP by measuring the emitted luminance. In some embodiments, the cAMP-dependent enzyme is adenylate cyclase, and the sample is contacted in the presence of pyrophosphate under conditions suitable for the synthesis of ATP by the adenylate cyclase. In some embodiments, the cAMP-dependent enzyme is a cAMP-dependent protein kinase, and the sample is further contacted with ADP and a phosphorylated substrate for the cAMP-dependent protein kinase under conditions suitable for the synthesis of ATP by the cAMP-dependent protein kinase. In some embodiments, the cAMP-dependent enzyme is a cAMP-dependent phosphodiesterase, and the sample is contacted in the presence of pyrophosphate and light under conditions suitable for the synthesis of ATP by the cAMP-dependent protein kinase, and the luminance emitted is measured in a dark chamber of a luminometer. In some embodiments, the cAMP is generated from the activity of a G-protein coupled receptor.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of a preferred mode of the present invention known as “DeathTRAK”. An important advantage of DeathTRAK is that all of the depicted events and reactions can take place in a single reaction vessel, and that a direct readout of the signal is obtained with no further sample processing (i.e., it is “homogeneous”). Abbreviations used in FIG. 1: NAD+: nicotinamide adenine dinucleotide (oxidized form); G3P: glyceraldehyde-3-phosphate; Pᵢ: phosphate ion; G3PDH: glyceraldehyde-3-phosphate dehydrogenase; NADH: nicotinamide adenine dinucleotide (reduced form), 1,3DPG: 1,3 diphosphoglycerate; ADP: adenosine diphosphate; PGK: phosphoglycerokinase; 3PG: 3-phosphoglycerate; ATP: adenosine triphosphate.

FIG. 2 shows the results of optimizing the DeathTRAK homogeneous cocktail, using the G3PDH test enzyme. Results with the unoptimized and optimized cocktails are shown (along with R² correlation values), using similar ranges of enzyme concentrations. Error bars are displayed but are too small to see.

FIG. 3 shows the results of cytotoxicity measurements of HL-60, compared with visual estimates of cell death.

FIG. 4 shows the dynamic range of the optimized DeathTRAK assay with lysed Raji cells. The response is nearly linear over four orders of magnitude. The graph is a log-log plot. Error bars are displayed but are too small to see, except for one side of the error bar at the lowest point (the other side of the error bar enters negative values and cannot he displayed on a low log plot).

FIG. 5 shows the results of enhancement of complement-mediated killing of the prostate cancer cell line PC-3 by an anti-Factor I antibody, as measured by the DeathTRAK homogeneous assay. The units of the Y axis are Relative Luminance Units/Second; the Y values were obtained by linear fits of the luminance data against the time that each luminance reading was taken (compare FIG. 6).

FIG. 6 shows the results of the same experiment as FIG. 5, using only a single data-point from each reaction for the analysis. The units of the Y axis are therefore Relative Luminance Units, reflecting an absolute luminance at a point 2.6 minutes into the DeathTRAK assay.

FIG. 7 shows the effect of additional adenosine diphosphate (ADP) on the response of the DeathTRAK homogeneous cocktail. ADP was added to three reactions, with various concentrations of PGK. Controls (marked “Original ADP”) had the same concentrations of PGK but received no additional ADP.

FIG. 8 shows an example of the lag phase, due to extended exposure of the reaction cocktail to light in the presence of PGK.

FIG. 9 shows an example of a reaction in which precautions were taken to eliminate the lag phase.

FIG. 10 shows a cytotoxicity assay in which live or dead E. coli cells (strain EV-5) were diluted directly from culture and mixed with the DeathTRAK homogeneous cocktail. Data from duplicate runs are shown.

FIG. 11 shows an example of protection of released G3PDH enzyme by a cocktail containing a combination of dithiothreitol and a mixture of protease inhibitors (available from Sigma as catalog #P-2714).

FIG. 12 shows the results of cytotoxicity/proliferation mode measurements made with the 841 CON cell line, using the detergent Nonidet-P40 as both the toxin and the total-lytic agent.

FIG. 13 shows the results of cytotoxicity mode measurements of the effects of three antibiotics, made with E. coli strain K1.

FIG. 14 shows the results of proliferation mode measurements made with E. coli strain K1, from the same experiment as the data of FIG. 13, following addition of the lytic agent, a polymyxin B/lysozyme combination.

FIG. 15 shows both cytotoxicity and proliferation data from an experiment similar to those shown in FIGS. 13 and 14, using gentamicin with the E. coli K1 strain.

FIG. 16 shows the results of cytotoxicity mode measurements made with Group-A Streptococcus, using three antibiotics as toxins.

FIG. 17 shows the results of proliferation mode measurements made with Group-A Streptococcus, from the same experiment as the data of FIG. 16, following addition of the detergent Nonidet P-40 as the total-lytic agent.

FIG. 18 is a schematic diagram of a preferred mode of the present invention known as “PhosTRAK,” a rapid, homogeneous, luminescent phosphatase assay. Abbreviations used in FIG. 18: NAD+: nicotinamide adenine dinucleotide (oxidized form); G3P: glyceraldehyde-3-phosphate; Pᵢ: phosphate ion; G3PDH: glyceraldehyde-3-phosphate dehydrogenase; NADH: nicotinamide adenine dinucleotide (reduced form); 1,3DPG: 1,3 diphosphoglycerate; ADP: adenosine diphosphate; PGK: phosphoglycerokinase; 3PG: 3-phosphoglycerate; ATP: adenosine triphosphate.

FIG. 19 demonstrates quantification of free phosphate using the PhosTRAK assay.

FIG. 20 demonstrates quantification of free phosphate using the PhosTRAK assay.

FIG. 21 shows a scheme of a representative method of the invention for measuring the catalytic activity of lactate dehydrogenase. Abbreviations used in FIG. 21: NAD⁺ and NADH represent the oxidized and reduced forms of nicotinamide adenine dinucleotide, respectively; G3P is glyceraldehyde-3-phosphate; Pᵢ is PO₄³⁻ or inorganic phosphate; 1,3DPG is 1,3-diphosphoglycerate or 1,3-diphosphoglyceric acid; ADP is adenosine diphosphate; ATP is adenosine triphosphate; 3PG is 3-phosphoglycerate or 3-phosphoglyceric acid; hv is light; LDH is lactate dehydrogenase; G3PDH is glyceraldehyde-3-phosphate dehydrogenase; PGK is phosphoglycerokinase.

FIG. 22 shows a scheme of a representative method of the invention for detecting and/or quantifying nitrate ions. Abbreviations used in FIG. 22: NAD⁺ and NADH represent the oxidized and reduced forms of nicotinamide adenine dinucleotide, respectively; G3P is glyceraldehyde-3-phosphate; Pᵢ is PO₄³⁻ or inorganic phosphate; 1,3DPG is 1,3-diphosphoglycerate or 1,3-diphosphoglyceric acid; ADP is adenosine diphosphate; ATP is adenosine triphosphate; 3PG is 3-phosphoglycerate or 3-phosphoglyceric acid; hv is light; G3PDH is glyceraldehyde-3-phosphate dehydrogenase; PGK is phosphoglycerokinase.

FIG. 23 shows a scheme of a representative method of the invention for measuring the activity of acetylcholinesterase. Abbreviations used in FIG. 23: AO is aldehyde oxidase, or any of many enzymes with NADH-dependent acetate reductase activity; NAD⁺ and NADH represent the oxidized and reduced forms of nicotinamide adenine dinucleotide, respectively; G3P is glyceraldehyde-3-phosphate; Pᵢ is PO₄³⁻ or inorganic phosphate; 1,3DPG is 1,3-diphosphoglycerate or 1,3-diphosphoglyceric acid; ADP is adenosine diphosphate; ATP is adenosine triphosphate; 3PG is 3-phosphoglycerate or 3-phosphoglyceric acid; hv is light; ACHE is acetylcholinesterase; AO is aldehyde oxidase, or an enzyme with NADH-dependent acetate oxidase activity; G3PDH is glyceraldehyde-3-phosphate dehydrogenase; PGK is phosphoglycerokinase.

FIG. 24 shows a scheme of another representative method of the invention for measuring the activity of acetylcholinesterase. Abbreviations used in FIG. 24: ACHE is the enzyme acetylcholinesterase; AK is an acetate kinase; ADP is adenosine diphosphate; ATP is adenosine triphosphate; hv is light.

FIG. 25 shows a scheme of another representative method of the invention for measuring the activity of acetylcholinesterase. Abbreviations used in FIG. 25: ACHE is the enzyme acetylcholinesterase; CK is an choline kinase; ADP is adenosine diphosphate; ATP is adenosine triphosphate; hv is light.

FIG. 26 shows a scheme of a representative method for the detection of cAMP. Abbreviations used in FIG. 26: cAMP is the test reagent cyclic AMP; PPi is pyrophosphoric acid, or a salt of pyrophosphate suitable for reaction with adenylate cyclase; ATP is adenosine triphosphate; hv is light.

FIG. 27 shows a scheme of another representative method for the detection of cAMP. Abbreviations used in FIG. 27: cAMP is the test reagent cyclic AMP; PPi is pyrophosphoric acid, or a salt of pyrophosphate suitable for reaction with adenylate cyclase; ATP is adenosine triphosphate; hv is light.

FIG. 28 shows a scheme of another representative method for the detection of cAMP. Abbreviations used in FIG. 28: PKA is protein kinase A, or another cAMP-dependent protein kinase; cAMP is cyclic AMP; PPi is pyrophosphate; ADP is adenosine diphosphate; ATP is adenosine triphosphate; hv is light.

### DETAILED DESCRIPTION OF THE PREFERRED EMBODIMENT

Unless specifically defined herein, all terms used herein have the same meaning as they would to one skilled in the art of the present invention. In one aspect, the present invention provides methods for measuring the amount or activity of a component in a sample using a luminescent assay. As used herein, the term “measuring” refers to making a single measurement, making multiple measurements, or monitoring by making continuous measurements. In addition, the term “measuring” encompasses both detecting the presence of a component and quantifying the concentration or quantity of a component. The term “amount” refers to either the concentration or the quantity (which may be zero or non-zero) of a component in the sample.

The luminescent assays used in the invention are performed under suitable conditions such that the luminance emitted from the sample depends on the amount of the component to be measured in the sample. As used herein, the term “luminance emitted from the sample depends on” a component refers to a relationship between the emitted luminance and the component such that the emitted luminance changes in response to changes in the amount of that component. For example, the emitted luminance may be proportional or inversely proportional to the amount of the component. Suitable conditions include the presence of appropriate buffer constituents, cofactors, and enzyme substrates. For example suitable conditions for the emitted luminance to depend on the amount of a component are conditions that include substrates, buffer components, and any other molecules necessary for the light-producing reaction of luciferase to occur or undergo a change in response to a change in the amount of a component that is measured by the assay. In some embodiments, the assays are performed under suitable conditions that permit all reactions necessary for production of light in response to the analyte to occur simultaneously. In some embodiments, the assays are performed under suitable conditions that permit the reactions to be carried out in a series of steps, which may include alterations in conditions to enable, enhance, or inhibit the activities of specific enzymes in order to enable or enhance the production of light in response to the analyte. Such altered conditions may include without limitation changes in temperature; pH; redox potential; buffer components; illumination; mechanical agitation; the presence of live or dead cells; or concentrations of specific substrates, enzymes, or other molecules.

In some embodiments, the methods of the invention comprise the use of at least one enzyme. As used herein, the term “at least one enzyme” refers to one or more enzymes and encompasses the use of a series of enzymes, in which at least one reaction product of an enzyme acting earlier in the series serves as a substrate for an enzyme acting later in the series. For example, at least one enzyme that results in the interconversion of NAD⁺ and NADH may comprise acetylcholinesterase and an NADH-dependent enzyme with acetate-reductase activity such as, for example, an NADH-dependent aldehyde oxidase. In this series of enzymes, acetylcholinesterase catalyses the conversion of acetylcholine to acetate and choline, and the NADH-dependent enzyme with acetate-reductase activity catalyses the production of NAD⁺ from NADH by reduction of the acetate produced in the acetylcholinesterase-catalyzed reaction.

Typically, a luciferase enzyme that is dependent on a high-energy molecule is used in the methods of the invention. High-energy molecules that may serve as luciferase substrates include, but are not limited to, ATP and NADH.

The present invention provides a variety of coupled luminescent methods and compositions for use in various assays, including for assaying cytotoxicity, membrane damage, cell proliferation, and enzymatic activity. Luminescent methods have an important advantage over other liquid-phase methods in that the sensitivity of luminescent detection of most phenomena is greater than the sensitivity of any other method. For example, electrochemiluminescent (ECL) analysis of Western blots is now the gold standard in sensitivity, and ECL methods are the most sensitive in enzyme immunoassays. “Coupled luminescent” methods are methods in which the activity of the enzyme or enzymes of interest is “coupled” in some manner to production or consumption of a high-energy molecule, such as ATP or NADH, which is a luminescent substrate for one or more of the biological luciferases. Luciferases are enzymes which produce light as they consume such high-energy molecules. Properly designed coupled luminescent assays are able to combine the advantages of specific assays for enzyme function with the very great sensitivity of luminescent detection methods. In these systems the inherent sensitivity of luciferase detection is enhanced by the “amplification” effect of enzyme turnover, which produces thousands, millions, or billions of high-energy molecules for each molecule of enzyme.

In one embodiment of the present invention, the measurement takes place in a one-step “homogeneous” system; a homogeneous system is one in which the sample is mixed with the reagent cocktail, and no separations or further transfers are required prior to readout. The enzyme or enzymes whose activity is being measured (in enzymatic activity mode) or the enzyme or enzymes released from cells (in cytotoxicity, membrane-damage, proliferation, or combined cytotoxicity/proliferation mode) are coupled in a single reaction vessel to production of ATP, NADH, or another high-energy molecule which is a substrate for a luciferase; the luciferase then produces light from the chemical energy of the high-energy molecule. The increase or decrease in the luminance signal is related to the concentration(s) of the enzyme or enzymes whose activity or activities are of interest. Taking cytotoxicity assays as an example, the reagent cocktail may be added to the cells under test before, after, or simultaneously with the potentially cytotoxic agent, depending on the kind of test being performed. If a quantitative determination of killing rate were desired, the cells could be mixed with the agent first and incubated for a fixed interval, after which the reagent cocktail would be added; this would provide an accurate picture of aggregate cell death over time. For maximum speed, reagent cocktail, cells, and the potentially cytotoxic agent could be mixed simultaneously; depending on the speed of killing, a signal could be obtained within minutes, or possibly even less than one minute. Finally, mixing the reagent cocktail with cells before addition of the potentially cytotoxic agent would allow comparison of the viability before and after treatment. These last two modes would also allow the user to follow the whole toxicity reaction in real time. A calibration standard of cells could be used to obtain absolute quantification. Note that the homogeneous nature of this aspect of the invention distinguishes it, in the case of cytotoxicity, from the GPL method, in which the assay reagents are not added in a single reagent mixture; instead the GPL method requires multiple transfers and incubations, first from the sample being tested to the “GP” cocktail; next, following an incubation, from the GP cocktail to the luciferase cocktail, which must also be aliquoted separately. Moreover, the GPL assay is not compatible with live cells, which must be separated by centrifugation, filtration, or another method before the first transfer. In the present invention, all constituents necessary for the assay are added in a single aliquot to the sample being tested, and there is no need to remove live cells from the supernatant.

In a related aspect, the present invention provides a set of methods for measuring cell proliferation. In this mode, the cells are killed by addition of a lytic agent before, after, or simultaneously with addition of the reagent cocktail. If the reagent cocktail is added before the lytic agent, a readout is obtained both of cells killed by processes under study (before addition of the lytic agent) and total cells present (after addition of the lytic agent). If the reagent cocktail is added after the lytic agent, a consistent increase or decrease in the luminance signal may he obtained, representing the total number of cells. If the lytic agent and reagent cocktail are added simultaneously, maximum throughput may be achieved, and the lytic process may be observed in real time (this is also true when the reagent cocktail is added first). Note that this feature also distinguishes the present invention from the GPL method. In the GPL assay, it is necessary to extract live cells from the sample being tested before addition of the GPL reagents, since in many cases these live cells could be killed by the GPL reagents. Thus failure to remove these live cells would lead to a mixed signal of actual cytotoxicity and a portion of the cells that were still alive prior to addition of the GPL reagents. The present invention does not suffer from this limitation, since the reagent mixture is compatible with all types of live cells that have been tested, including several mammalian cell lines, and Gram positive and Gram negative bacteria.

In a preferred embodiment, the present invention provides a set of methods for measuring cell proliferation and cytotoxicity in the same experiment, in a simple, two-step process which maintains the homogeneous nature of the assay. The reagent cocktail is added to cells before, during, or after initiation of the cytotoxic process. Following an incubation to obtain a luminance increase or decrease to obtain a cytotoxicity readout (typically 0.5 to 10 minutes), the lytic agent is added. The luminance increase or decrease following addition of the lytic agent represents the total biomass, alive and dead, at the time of the assay (proliferation readout). The live biomass (as of the time immediately before the lytic step) may generally be calculated by subtracting the toxicity readout from the total-biomass signal. The option of measuring both cytotoxicity and proliferation (or viability) in the same sample distinguishes the present invention from other available liquid-phase cytotoxicity and proliferation assays.

In another aspect, the present invention provides a set of methods and compositions for killing live cells of various types in a manner consistent with accurate reading of luminance due to enzyme release after the lytic step.

In another aspect, the present invention provides a set of methods and compositions for protecting an oxidation- and/or proteolysis-sensitive enzyme released by dying cells from oxidation and/or proteolysis during an initial incubation period, such that enzyme released by cells that die during the incubation period will be measurable at the end of that period.

In another aspect, the present invention provides a set of methods for detecting membrane damage, with or without associated cytotoxicity. Membrane damage associated with cell death is detected as cytotoxicity as discussed above. Membrane damage can also be detected separately from cell death (i.e., non-fatal damage) by performing assays by one of the specified methods for enzyme release, followed by an optional recovery phase and subsequently by a proliferation or viability assay, such as the CFU assay, a metabolism-based assay, or the proliferation mode of the present invention.

In another aspect, the present invention provides a set of methods of detecting enzymatic activity by coupling the enzymatic activity to production or consumption of a high-energy molecule that is a luciferase substrate.

In another aspect, the present invention provides a set of methods for optimizing a coupled luminescent assay for (1) time linearity, (2) linearity with enzyme or cell number to be measured, (3) compatibility with cells of various types, (4) homogeneous use, and (5) use in high-throughput screening (HTS).

In another aspect, the present invention provides a set of methods for optimizing the storage conditions of reaction cocktails and reaction cocktail ingredients with respect to physical form, storage of mixed or separate ingredients, temperature of storage, and time of storage.

In another aspect, the present invention provides a set of methods for automatic reduction of complex data by linear regression. These methods compute linear fits for all possible time ranges within a given data set and (1) report slopes and/or correlations for all ranges, and/or (2) select the time range or ranges with the highest correlation(s) and report these ranges, correlations, and slopes, and/or (3) select a time range or ranges with certain given fit characteristics from a given sample or set of samples (which could be calibration or other standards) and apply that range or those ranges to all or a subset of the remaining samples, and/or (4) detect and report exceptional aspects of data obtained from a given sample or samples, or from the entire run, such as poor signal strength, linearity, time correlation, or correlation with expected values, and/or (5) evaluate the characteristics of the run, such as linearity and/or signal strength, and either make an automated decision to stop or continue reading the samples or report the run characteristics to the user to allow the user to make that decision.

In another aspect, the present invention provides a set of methods of measuring cytotoxicity, membrane damage, cell proliferation, and enzymatic activity with the use of a stop reagent. The increase or decrease in the luminance signal is wholly or partially stopped by the reagent, allowing the user to treat the ending luminance value as the readout of the assay, such endpoint reading to take place at any time convenient to the user.

In another aspect, the present invention provides a set of methods for HTS of compounds for any of a number of desirable or undesirable characteristics: (1) desirable cytotoxicity against an identified target, which may be a cancer cell type or an infectious microorganism; (2) undesirable cytotoxicity against normal cell types in a drug candidate; (3) growth-affecting characteristics; (4) membrane damage; or (5) inhibitory or rate-enhancing properties in a given enzymatic system. These methods involve preformulation of the reaction cocktail and preloading this cocktail into an injector of a luminometer, followed by homogeneous or non-homogeneous assay of the rate of increase or decrease in the luminance signal and either automated data reduction, non-automated data reduction, or the use of a stop reagent and a single readout. The HTS run may be (1) terminated after a single or fixed number of reads, (2) terminated automatically when certain criteria are achieved, or (3) terminated at the user's discretion.

In another aspect, the present invention provides a set of methods for testing an individual patient's cancer tumor cells or infecting microorganisms for sensitivity or resistance to a potential drug, drug mixture, or panel of drugs or drug mixtures.

In another aspect, the present invention provides a set of methods for detecting and quantifying apoptosis (programmed cell death). This may be accomplished as tinder the description of cytotoxicity measurement, above, or by coupled luminescent detection of the increase levels of nuclear G3PDH associated with apoptosis, or by a combination of these methods.

In another aspect, the present invention provides a set of methods for detecting the presence of live cells in environments that are intended to be sterile or have low bioburdens. This would be accomplished by taking a sample (either a liquid sample or a swabbed sample, which can then be transferred or washed into a liquid sample), using a lytic agent, and performing a coupled luminescent assay as described elsewhere under proliferation assays.

In another aspect, the present invention provides a set of methods for very sensitive detection of environmental toxins. This would be accomplished by mixing an environmental sample, such as an aliquot of seawater or residue from a wash of shellfish or other food samples, with a coupled luminescent reaction cocktail in the presence of a cell type known to be sensitive to the toxin in question, and measuring the resulting cytotoxicity.

In another aspect, the present invention provides a set of methods of detecting and/or quantifying free phosphate by coupling the presence of free phosphate to production of ATP via the activity of G3PDH and PGK, which are both supplied in the reagent mixture. Detection and/or quantification of free phosphate is of importance in biochemistry, enzymology, environmental science, and other areas.

In a second preferred embodiment, the present invention provides a set of methods for detecting the enzymatic activity of a phosphatase by quantifying the phosphate produced by the reaction of the phosphatase, which is accomplished by coupling the presence of free phosphate to production of ATP via the activity of G3PDH and PGK, which are both supplied in the reagent mixture. In this embodiment, the present invention enjoys a number of advantages over other phosphatase assays in current use, including great speed, extreme simplicity of operation, and the ability to use natural substrates, or, when they are unavailable, appropriately chosen phosphorylated peptide or protein substrates, or other phosphorylated molecules as similar as is practicable to the in vivo substrates.

In another aspect, the present invention provides a set of methods for detecting activity of intracellular phosphatases by optionally measuring phosphatase activity by the method described above before lysis, lysing the cells by one of the methods provided in the invention or by another method, and again measuring phosphatase activity. The principle may also be applied to measurement of phosphatase activity inside particular cellular organelles.

In another aspect, the present invention provides a set of methods for measuring activity of specific phosphatases, for which specific substrates are available, against a background of other phosphatases and/or free phosphate by measuring the quantity of phosphate present or the rate of phosphate production by the methods provided, adding the specific substrate or substrates, and again measuring the quantity of phosphate present (after a time interval of the user's choice) or the rate of phosphate production.

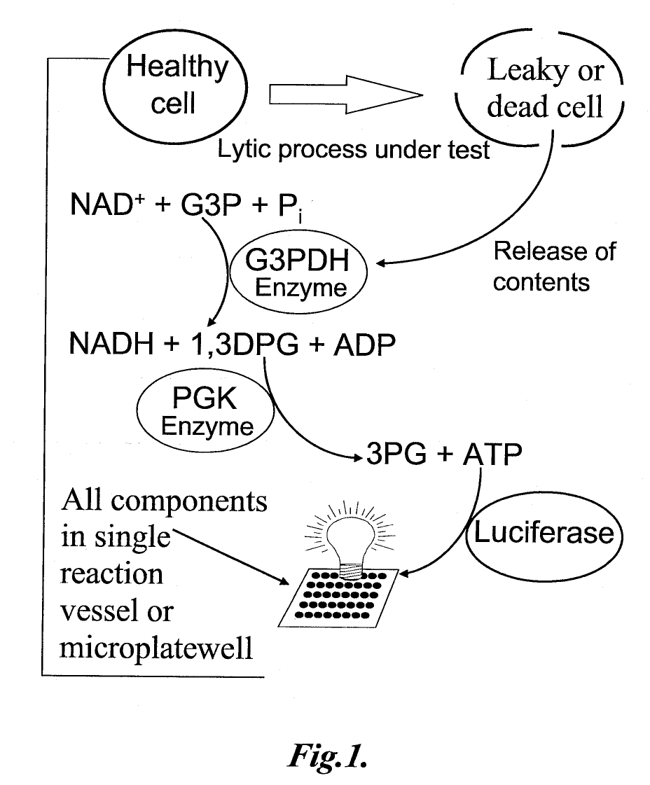

Accordingly, one aspect of the present invention provides methods of measuring cytotoxicity. In a preferred embodiment, cytotoxicity is measured in a homogeneous assay in a microplate luminometer. The luminance signal is produced by firefly luciferase acting on adenosine triphosphate (ATP), which in turn is produced by the coupled reactions of glyceraldehyde-3-phosphate dehydrogenase (G3PDH) and phosphoglycerokinase (PGK), two consecutive enzymes of the glycolytic pathway. G3PDH, a very abundant enzyme in all known cells, is measured to quantify release (and therefore cell death and/or membrane damage), while PGK, which is generally not so abundant in cells, is supplied in the reaction cocktail, along with glyceraldehyde-3-phosphate (G3P), nicotinamide adenine dinucleotide oxidized form (NAD+), inorganic phosphate (Pi), dithiothreitol (DTT), adenosine diphosphate (ADP), the components of the luciferase reaction, and appropriate buffers and salts (see FIG. 1 for a schematic diagram of the assay, and EXAMPLE 1 for additional details of the components). Essential to the invention is the fact that G3PDH is abundant in all living cells; therefore the user can be confident that the invention will be useful in measuring cytotoxicity and/or proliferation of a specific cell type without prior testing. Moreover, G3PDH is a natural component of the cells, and does not need to be introduced into the cells in any manner. This distinguishes the present invention from all methods which require prelabeling of the cells, or transfection, transformation, or other methods of introducing proteins or other molecules into the target cells in order to generate a signal in a later step.

It should be noted that as with any assay method, the methods of the present invention are subject to incorrect results if certain substances which interfere with the assay components are present. As an example, if a user is employing one of the modes herein described for screening a compound library for cytotoxic effects, and one of the compounds in the library happens by chance to he an inhibitor of one of the enzymes essential to operation of the mode in use, an incorrect signal may be obtained. As in all screening studies, it is desirable to follow up screening runs with further experiments using independent methods. However, the range of substances of interest that interfere with DeathTRAK is likely to he far smaller than with the MTT and other metabolic assays, as pointed out above. Likewise, the ATP-release assay is vulnerable to compounds that interfere with luciferase activity, as well as the whole set of agents that affect the ATP charge of living cells.

EXAMPLE 1 shows an assay of the effects of a cytotoxic agent on cells derived from human prostate cancer. In this case the cytotoxic agent is the alternative pathway of complement, but it may be a candidate drug molecule, food additive, environmental sample, or any other substance or mixture in liquid form with the potential to cause cytotoxicity or membrane damage. The experiment was performed to test the effects of a monoclonal antibody directed against complement Factor I (FI) on complement-mediated lysis of the PC-3 cell line.

A preferred mode of the invention involves the simultaneous reaction of three enzymes, while maintaining compatibility with live cells, protecting the G3PDH enzyme from inactivation or denaturation, and allowing individual measurements of both cytotoxicity and proliferation in the same sample. The process of meeting the requirements of the enzymes and cells is described in EXAMPLE 2, while the combined cytotoxicity/proliferation mode is described in EXAMPLE 8, below. Under EXAMPLE 2, the process of finding a buffer that yielded high signal strength and was compatible with live cells was carried out, concentrations of PGK and ADP were optimized; and a comparison of time-linear fits with single-point readouts shows that single data points may be used effectively to report DeathTRAK results, after as little as 2.6 minutes. It was necessary to adjust the concentrations of various reagents in order to obtain satisfactory performance while meeting the various constraints imposed by the system. Examples of such constraints are: (1) the assay cocktail must be homogeneous; i.e., after the cocktail is loaded into the injector, the only mixing step should be the automated injection of cocktail into sample, with no separations needed; (2) the cocktail must not significantly damage live cells in the time-frame of the assay; (3) the cocktail must contain necessary reagents in concentrations adequate to support strong signals and/or extended reactions, without yielding excessive background signals; (4) the time-dependent response of the assay must be as near linear as possible, over as wide a dynamic range as possible; and (5) storage characteristics of the assay cocktail must be satisfactory for widespread use. Examples of constraints encountered within category (3) include: (A) the phosphate ion is necessary for G3PDH activity and must be present at adequate levels, but it is a potential inhibitor of enzymes which metabolize ADP/ATP, and its level must therefore be kept in check; (B) the PGK enzyme is essential for production of ATP, and in many cases it is a limiting reagent (see EXAMPLE 2B); however preparations of PGK are almost always contaminated with small amounts of G3PDH, which adds to the dynamic background signal during the reaction, and also tends to convert G3P to 1,3 DPG during storage, contributing to static background; and (C) ADP is an essential component and is often limiting (see EXAMPLE 2C), but ADP preparations are often contaminated with ATP, which contributes directly to the static background.

EXAMPLE 2A shows the process of developing a cocktail which is compatible with live mammalian cells and shows an adequate response to the test enzyme G3PDH. The IMDM growth medium and phosphate-buffered saline (PBS) are both nearly isotonic with respect to mammalian cells and were chosen for a titration assay to determine the point at which the assay response would be optimal. Since phosphate ion is a potentially exhaustible component, concentrations with greater phosphate concentrations were preferred in cases of similar performance.

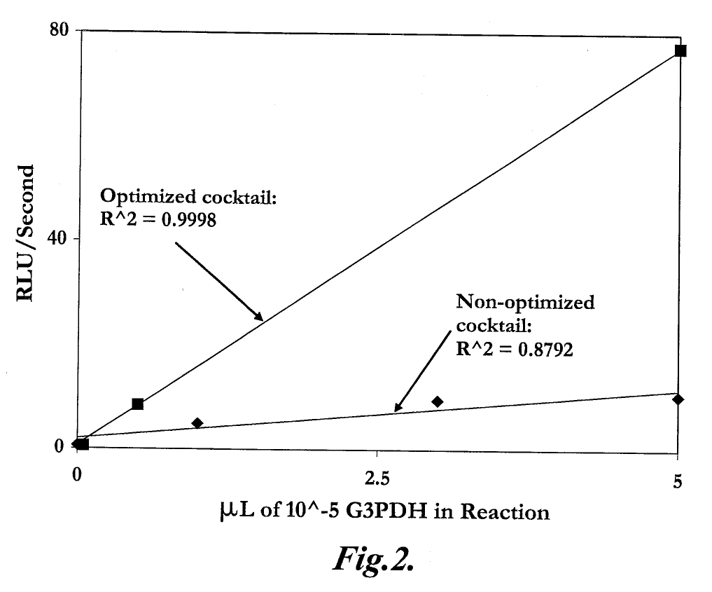

After the optimization process of EXAMPLE 2A the reaction cocktail was compatible with live cells, but exhibited only modest linearity and sensitivity with the test enzyme. FIG. 2 shows a comparison of the linearity and sensitivity before and after optimization steps represented by EXAMPLES 2B and 2C. Errors (standard deviations) are shown in this graph but are too small to be visible. The R2 was improved by optimization from 0.8792 to 0.9998, while the luminance response per unit enzyme was enhanced by over 7-fold. The assay of the optimized cocktail was performed with ten-fold serial dilutions. The significance of this plot is not merely the fact that the linear correlation is vastly improved, but also the fact that the line passes precisely through the origin, indicating an excellent proportionality between the amount of enzyme added and the signal response. EXAMPLE 2B shows the first of these optimization processes. As explained above, PGK is an important component of DeathTRAK, but if it is present in excess, then contaminating G3PDH adds to both static and dynamic background signals. PGK was therefore titrated over two orders of magnitude to determine how much greater a concentration could be present without unacceptable effects on the background and assay linearity.

The results of PGK optimization dramatically improved the response of DeathTRAK, but the improved signal exacerbates the saturation problem that is seen in the unoptimized cocktail even at moderate signal strength. EXAMPLE 2C shows that an increased starting concentration of ADP led to an enhanced signal.

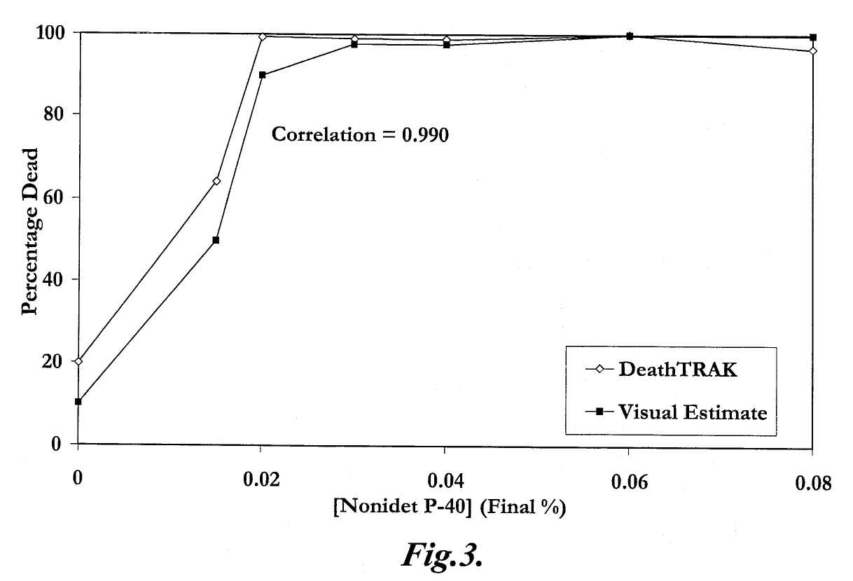

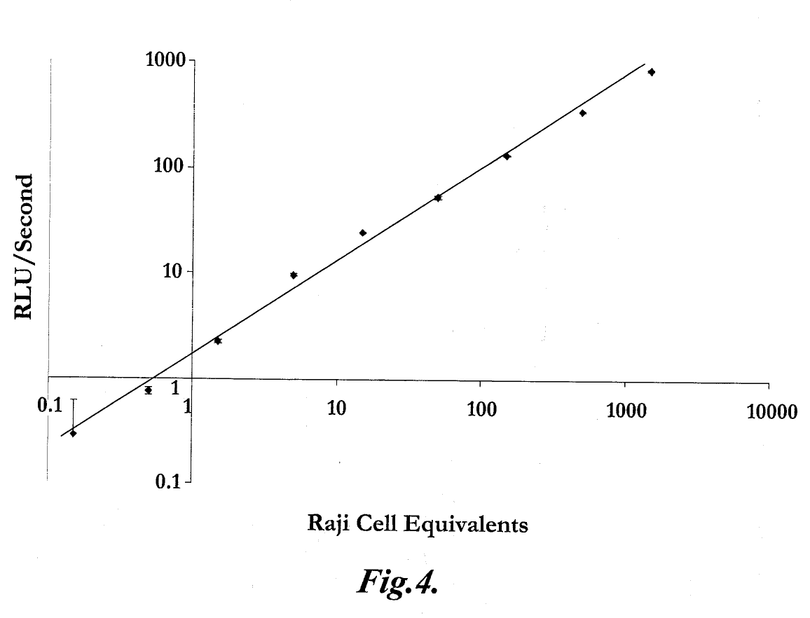

EXAMPLE 2D shows the results of various tests of the optimized cocktail. FIG. 3 shows the results of an experiment in which the DeathTRAK results are compared with another method (visual inspection) of determining cytotoxicity, yielding a correlation coefficient between the two methods of 0.990. This high degree of correlation between the results of measuring cytotoxicity by the methods of the present invention and the very distinct method of direct visualization adds to the confidence level and value attached to the invention. FIG. 4 shows the data obtained from Raji cells that were intentionally lysed prior to the assay, demonstrating a good response over four orders of magnitude. A highly reproducible signal was also obtained with the G3PDH test enzyme.

In another aspect, the present invention provides methods and compositions for optimizing stability during storage of a reaction cocktail for use in a coupled luminescent assay. EXAMPLE 2E shows the effects of lyophilization vs. freezing of the components in various combinations. This shows that freezing is a superior means of storage. EXAMPLE 2F is a test of the effect of frozen storage vs. storage at 4° C., including especially influence on the static background signal. Again, freezing proved to be a much better method of storage.

EXAMPLE 2G shows the lag phase that was experienced when the homogeneous assay was first used and provides methods for overcoming this problem.

In another aspect, the present invention provides methods and compositions for a single-timepoint coupled luminescent assay, making use of a stop reagent to end the production of the high-energy molecule that is the luciferase substrate, while allowing the luciferase reaction to continue. In a preferred embodiment of this concept, an inhibitor of G3PDH or PGK is used to stop production of ATP in the DeathTRAK reactions of EXAMPLES 1 and 2. This results in a fairly constant luminance signal and permits the user to read a single number at the end of a fixed interval, rather than having to deal with data reduction of a time-dependent signal. It is also possible to perform a single read without a stop reagent, but the stop reagent allows the read to be done at a time convenient to the user, multiple times, or for an extended period. EXAMPLE 2H demonstrates the use of a stop reagent with the DeathTRAK assay. Other candidate stop reagents are the synthetic peptide MEELQDDYEDMMEEN-NH2, which was derived from the N-terminus of human erythrocyte anion transporter, band 3 (Eisenmesser E Z and Post C B, Biochemisty 1998 Jan. 20; 37(3):867-77); vanadate ion (Crans D C, Simone C M, Biochemistry 1991 Jul. 9; 30(27):6734-41); iodoacetic acid (Baker M S, Bolis S, Lowther D A, Agents Actions 1991 March; 32(3-4)299-304; Rego A C, Areias F M, Santos M S, Oliveira C R, Neurochem Res 1999 March; 24(3).351-8), pentalenolactone (Ikeda M, Fukuda A, Takagi M, Morita M, Shimada Y, Eur J Pharmacol 2001 May; 411(1-2):45-53); acrylamide (Anuradha B, Varalakshmi P, J Appl Toxicol 1999 November-December; 19(6).405-9); 3-chloro-1-hydroxypropanone (Jones A R, Reprod Fertil Dev 1997; 9(6):577-81); koningic acid (Nakazawa M, Uehara T, Nomura Y, J Neurochem 1997 June; 68(6):2493-9); (S)-3-chlorolactaldehyde (Jones A R, Porter L M, Reprod Fertil Dev 1995; 7(5):1089-94); 3-bromo-1-hydroxypropanone (Porter L M, Jones A R, Reprod Fertil Dev 1995; 7(1):107-11); various phosphorylated epoxides and alphaenones (Willson M, Lauth N, Perie J, Callens M, Opperdoes F R, Biochemistry 1994 Jan. 11; 33(1).214-20); various phosphonates (Li Y K, Byers L D, Biochim Biophys Acta 1993 Jun. 24; 1164(1):17-21; however these compounds might also inhibit luciferase); or other compounds in the literature, some of which were developed as potential therapeutic agents for trypanosomiasis. Any molecule which inhibits G3PDH and/or PGK, but inhibits luciferase to a lesser or insignificant degree, might be used.

In another aspect, the present invention provides automated methods for analyzing data obtained from coupled luminescent reactions. Since these reactions are due to continuous enzyme activity, the luminance signal continues to increase with time during the reaction, unless a stop reagent is used. Thus there are several methods of reducing the luminance data to a single value, which may represent either a rate (change in luminance per second, commonly reported as Relative Luminance Units or RLU/second) or an absolute luminance level, read after a precise length of time, and/or with the use of a stop reagent. The case in which the readout is a single, absolute luminance level requires little additional analysis (although some aspects of the present invention could be used as a quality-assurance procedure even in these cases). The calculation of rates from time-dependent luminance data is described in more detail. Ordinarily, it will he possible and optimal to use the data from 0-3 or 1-3 minutes after reaction initiation for linear regression, since little or no saturation is usually evident in this time range unless the signal is extremely strong. However, when the user is dealing with samples about which very little is known, which may contain very large numbers of cells or an unexpectedly large proportion of dead cells, it is possible that the linear range of the assay will be exceeded even in this timeframe. One method of dealing with this potential problem is to select a useful time range of data and perform linear regression only within that time interval, but the selection of an appropriate range may be problematic, time-consuming, or subjective. Software may be used to provide a solution to this problem by analyzing every possible sequence of four or more consecutive data points within the data-set and selecting the time range with the highest coefficient of correlation. The program may report the linear fit for that optimal time range, the correlation obtained for that fit, and the actual timepoints that were used. It will be evident to one skilled in the art how to extend this program to a data-set larger than ten timepoints. Simple modifications to this program would allow the user to perform manipulations over all samples at once. For example, the program could be modified to choose the most linear range from a particular standard or calibration sample (or the average of a subset of the samples) and calculate the rate using that same time range for all the samples. Alternatively, it will be evident to one skilled in the art that a closely related procedure could find a time range which yielded the best minimum correlation over the entire sample set and applied that time range to all samples. These latter methods have the advantage that the same time range is used for all samples. Moreover, various warnings could be added to the code, such as “no good fit exists,” “data non-monotonic,” “slope outside expected range” (especially for standards and calibrators), “lag phase encountered,” or “saturation reached.”

In another aspect, the present invention provides methods and compositions for measuring bacteriolysis. This is shown in EXAMPLE 4. The combined cytotoxicity/proliferation mode is shown in use with bacteria under EXAMPLE 8.

In another aspect, the present invention provides methods and compositions for measuring cytotoxicity and/or proliferation by means of combinations of enzymes other than those shown in FIG. 1. As an example, the Aldolase-DeathTRAK reaction is similar to the DeathTRAK reaction, but glyceraldehyde-3-phosphate is generated in situ, by the action of aldolase on fructose-1,6-bisphosphate, rather than being provided in the cocktail as it is in DeathTRAK. EXAMPLE 5 demonstrates the use of this alternative assay and shows the general applicability of the coupled-luminescent concept to various enzyme combinations, as discussed further under EXAMPLE 14.

In another aspect, the present invention provides methods and compositions for measuring cytotoxicity of a compound, mixture of compounds, cell or cell fragment, virus or viral fragment, organism or organismal fragment, radiation, physical or mechanical stress, or any other substance, process, or combination of these. In general the cytotoxic or damaging effects of substances or processes that are compatible with a liquid phase can be measured in the same manner as in EXAMPLE 1. The DeathTRAK assay, Aldolase-DeathTRAK assay, and other coupled luminescent systems as exemplified below are compatible with a wide range of substances, buffers, lytic agents, and cell types. EXAMPLE 6 describes the general case of measurement of cytotoxicity or membrane damage induced by a “cytotoxic agent,” which may he any of the entities listed in this paragraph.

In another aspect, the present invention provides methods and compositions for measurement of cell proliferation (see EXAMPLE 7). These methods involve either killing of all the cells or introduction of a substance or process which induces release of G3PDH from the cells, accompanied by a DeathTRAK assay or one of the other coupled luminescent assay types described under EXAMPLE 14.

In a preferred mode of use, the present invention provides methods and compositions for measuring both cytotoxicity and proliferation (or viability) of a single sample. This involves a combination of the methods described under EXAMPLES 6 and 7. The combined method is described in EXAMPLE 8, including experiments with both mammalian cells and bacteria.

In another aspect, the present invention provides methods and compositions for high-throughput screening for cytotoxicity and/or membrane damage and/or proliferation (EXAMPLE 9). The cytotoxicity/membrane damage may be desirable (as in screening for drug candidates with activity against a given cell type, such as a cancer cell or infectious organism) or undesirable (as in screening lead compounds or libraries for undesirable effects).

In another aspect, the present invention provides methods and compositions for screening for drug sensitivity and drug resistance (EXAMPLE 10). These methods may be used, for example, to aid decisions as to treatment strategy for a patient who is suffering from cancer or an infectious disease.

In another aspect, the present invention provides methods and compositions for research into and/or measurement of apoptosis (EXAMPLE 11).

In another aspect, the present invention provides methods and compositions for testing for the presence of live cells in cases where sterility or a low bioburden is desirable (EXAMPLE 12).

In another aspect, the present invention provides methods and compositions for environmental toxicity testing (EXAMPLE 13).

In another aspect, the present invention provides methods and compositions for extension of coupled luminescent assays to other enzyme systems (EXAMPLE 14).

In another aspect, the present invention provides methods and compositions for extension of coupled luminescent assays to applications other than cytotoxicity and proliferation (EXAMPLE 15).

In another aspect, the present invention provides methods and compositions for quantifying free phosphate. In a second preferred mode, the total quantity, total change, or rate of change in the amount of free phosphate is used as an indication of the activity of a phosphatase or phosphatases, and may be used in screening for inhibitors or other modulators of phosphatase activity (EXAMPLE 16).

In another aspect, the present invention provides alternative applications for measurement of free phosphate as described in EXAMPLE 16. These alternative applications are described in EXAMPLE 17.

In the examples described further below, assays with specific parameters are exemplified. However, the present invention provides a set of methods and compositions for coupled luminescent assays using various concentration ranges of the chemical and biochemical components specified for the DeathTRAK assay described herein, such that the assay functions with the following concentrations:
Dithiothreitol (DTT): 0-20 mM final concentration;
Adenosine diphosphate (ADP): 0-1 mM final concentration, or alternatively, ultrapurified ADP: 0-1 mM final concentration;
Nicotinamide adenine dinucleotide, oxidized form (NAD+): 0.1-50 mM final concentration;
Glyceraldehyde-3-phosphate: 1 mM-100 mM final concentration;
Triethanolamine: 0-1M final concentration;
Sodium phosphate: 0.1 mM to 1 M final concentration;
Ethylamine diamine tetraacetic acid: 0-50 mM final concentration;
Bovine serum albumin: 0-20 mg/mL final concentration;
ATP assay cocktail: 1-85% final concentration;
ATP assay diluent: 0-90% final concentration;
Phosphoglycerokinase (PGK): 1 in 10¹¹ parts to 1 in 10⁵ parts final concentration (beginning with stock solution at approximately 5000 units per mL), or alternatively, ultrapurified PGK: 1 in 10¹¹ parts to 1 in 10³ parts final concentration (beginning with stock solution at approximately 5000 units per mL);
IMDM-0-80% of the final reaction volume;
PBS-0-80% of the final reaction volume.

As explained herein, data may be obtained from the DeathTRAK assays and other assays based on the coupled luminescent principle at a single timepoint, at multiple individual timepoints, or as a time-linear fit of luminance data. The readout may be taken as soon as 1-2 seconds after injection, or as long as 24 hours after injection. DeathTRAK and other assays based on the coupled luminescent principle may be run at any temperature from just above freezing (0° C.) to approximately 60° C. Reaction cocktails and other components of DeathTRAK and other assays based on the coupled luminescent principle may be stored under a variety of conditions. In some cases the user may decide to use given storage conditions for convenience with full knowledge that part of the activity may be lost, since the sensitivity of the assay methods is so great that the remaining activity may be sufficient for many uses. The DeathTRAK reaction cocktail may be stored at room temperature for up to 12 hours, at 4° C. for up to 7 days, or at −15° C. or lower temperatures for up to five years. If the luciferase (ATP assay cocktail) component is kept lyophilized at −15° C. or colder and the PGK component is stored separately at 4° C., the reaction cocktail may be stored at 4° C. for up to one year.

The user has the option of using various types of microplates for obtaining luminance readouts. For example, standard luminance plates (black, white, mixtures of colors, or clear multi-purpose plates), tissue-culture plates, fluorescence plates, or EIA plates may be used. In contrast to methods that do not yield strong signals, the sensitivity of the coupled luminescent assay methods described herein is such that the signal obtained from all of these types of plates will be sufficiently strong for many uses. In particular, the cytotoxicity/proliferation dual mode experiments shown in EXAMPLE 8 were carried out in standard white luminance plates. The cells were seeded directly into the plates, and no further processing was needed prior to addition of the toxins under study the following day.

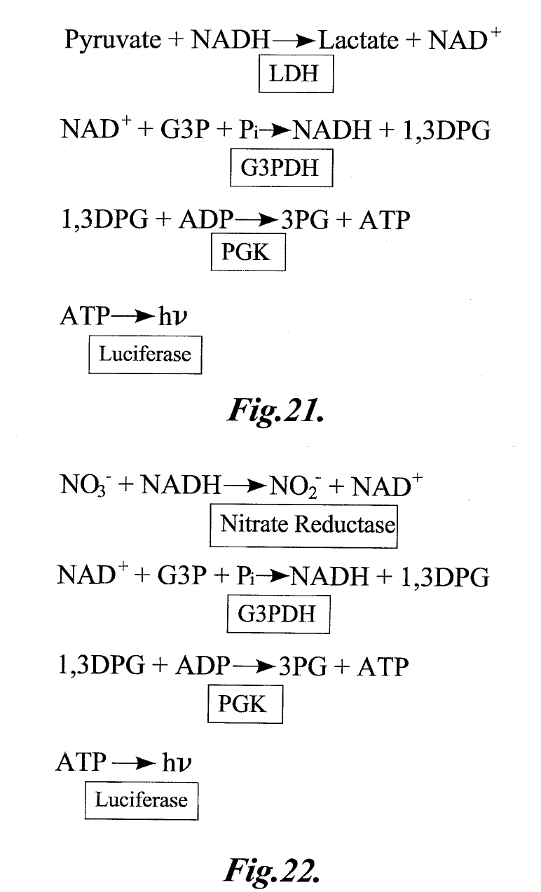

In another aspect, the present invention provides a set of methods of detecting and/or quantifying the enzymatic cofactor NAD⁺ (nicotinamide adenine dinucleotide, oxidized form) by coupling the presence of NAD⁺ to production of ATP via the activity of G3PDH and PGK, which are both supplied in the reagent mixture. The reaction scheme is very similar to that depicted in FIG. 1, but for detection of NAD⁺, G3PDH becomes a supplied reagent, while NAD⁺ is omitted from the reaction, so that NAD⁺ becomes a limiting reagent, and the light output is therefore sensitive to the concentration of this limiting reagent. The schemes in FIGS. 21 and 22 give examples of applications of NAD⁺ detection in assays of the enzymatic activity of lactate dehydrogenase and detection of the nitrate ion, respectively. Detection of NAD⁺ is generally of importance in biochemistry, enzymology, medicine, and other areas.

In another aspect, the present invention provides a set of methods of detecting and/or quantifying the enzymatic cofactor NAD⁺ (nicotinamide adenine dinucleotide, oxidized form) or NADP⁺ (nicotinamide adenine dinucleotide phosphate, oxidized form) by coupling the presence of NAD⁺ or NADP⁺ to production of ATP via the activity of an enzymatic reaction or series of enzymatic reactions, such that the production of ATP is dependent on the amount of NAD⁺ present in the reaction, and by then quantifying the ATP thus produced by measuring the light production of a luciferase. The schemes in FIGS. 21 and 22 are examples of applications of NAD⁺ detection by this method to in assays of the enzymatic activity of lactate dehydrogenase and detection of the nitrate ion, respectively.

In another preferred embodiment, the present invention provides a set of methods of measuring the catalytic activity of an enzymes or combination of enzymes that modulates the concentration or quantity of NAD⁺ or NADP⁺ by contacting the enzyme or enzymes with one or more of the reagent mixtures described above for detecting and/or quantifying NAD⁺ or NADP⁺. This modulation may be positive (i.e., the enzymatic activity increases the concentration of NAD⁺ or NADP⁺) or negative. The latter may be observed and/or measured by following the decrease in light output from the luciferase reaction as NAD⁺ or NADP⁺ is consumed. The schemes in FIGS. 21 and 22 are examples of measurement of the enzymatic activity of lactate dehydrogenase by this method and of quantification of nitrate by this method, respectively. It will be evident to one skilled in the art that the reaction series of the scheme in FIG. 22 may also be used to measure the activity of nitrate reductase, and therefore may serve as a separate example of the measurement of enzyme activity by this method. Enzymes which modulate the concentration or quantity of NAD⁺ or NADP⁺ are extremely numerous and many are of great biological and/or scientific importance. Such enzymes include many oxidases, dehydrogenases and reductases, epimerases and other isomerases, enzymes involved in energy production, and others.

In another aspect, the present invention provides a set of methods of measuring the catalytic activity of the enzyme lactate dehydrogenase (LDH) by supplying NADH (nicotinamide adenine dinucleotide, reduced form) and pyruvate or pyruvic acid in the reaction mixture, along with G3PDH, PGK, luciferase, and appropriate substrates for each with the exception of NAD⁺ and ATP (which are limiting reagents and are omitted), and measuring light emission by luciferase. This reaction series is depicted in the scheme shown in FIG. 21. In this reaction series, pyruvate represents pyruvic acid, sodium pyruvate, potassium pyruvate, or another salt of pyruvate suitable for reaction with LDH, or a combination of the acid and such a salt. Pyruvate may also be supplied by an enzymatic reaction or series of enzymatic reactions, either within the same reaction vessel or in a prior reaction step. Similarly, lactate represents lactic acid, or a salt of lactic acid, or a combination of the acid and a salt. NAD⁺ and NADH represent the oxidized and reduced forms of nicotinamide adenine dinucleotide, respectively. G3P is glyceraldehyde-3-phosphate. Pᵢ is PO₄³⁻ or inorganic phosphate, supplied as sodium phosphate, phosphoric acid, or another suitable salt of phosphate. 1,3DPG is 1,3-diphosphoglycerate or 1,3-diphosphoglyceric acid. ADP is adenosine diphosphate. ATP is adenosine triphosphate. 3PG is 3-phosphoglycerate or 3-phosphoglyceric acid. hv is light. The enzymes employed are specified within boxes below the reactions they catalyze, respectively. LDH is lactate dehydrogenase. G3PDH is glyceraldehyde-3-phosphate dehydrogenase. PGK is phosphoglycerokinase. Luciferase is firefly luciferase, beetle luciferase, or another ATP-dependent luciferase. In a reaction series intended to measure the quantity and/or activity of LDH, the reagents LDH, NAD⁺, and ATP are omitted or supplied in limiting quantities, while pyruvate, NADH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate. In a reaction series intended to measure the quantity of pyruvate, the reagents pyruvate, NAD⁺, and ATP are omitted or supplied in limiting quantities, while LDH, NADH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate. In a reaction series intended to measure the combined quantities of NAD⁺ and NADH, the reagents NAD⁺, NADH, and ATP are omitted or supplied in limiting quantities, while LDH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate.

In another aspect, the present invention provides as set of methods of measuring the catalytic activity of the enzyme lactate dehydrogenase (LDH) by coupling the presence of NAD⁺ or NADP⁺ to production of ATP via the activity of an enzymatic reaction or series of enzymatic reactions, such that the production of ATP is dependent on the amount of NAD⁺ or NADP⁺ present in the reaction, and by then quantifying the ATP thus produced by measuring the light production of a luciferase.

In another aspect, the present invention provides a set of methods of measuring cell lysis and/or membrane rupture or damage by measuring activity of LDH released by cells with damaged membranes by one or more of the methods described above.

In another preferred embodiment, the present invention provides a set of methods of measuring cell lysis and/or membrane rupture or damage by measuring the concentration or quantity of NAD⁺ generated as the LDH released by cells with damaged membranes is allowed to react in the reaction series described in the scheme shown in FIG. 21. Such a release assay may be used to measure membrane damage brought about by a process under test, to count cells after intentional cell lysis, or to measure both membrane-damaged cells and the total viable cell count.

In another aspect, the present invention provides a set of methods of measuring cell lysis and/or membrane rupture or damage by measuring the concentration or quantity of NAD⁺, NADP⁺, NADH, or NADPH released by cells with damaged membranes, using one of the methods described above for quantification of NAD⁺, NADP⁺, NADH, or NADPH.

In another aspect, the present invention provides a set of methods of detecting and/or quantifying the nitrate ion (NO₃⁻) by coupling the presence of nitrate to production of ATP via the activity of an enzymatic reaction or series of enzymatic reactions, such that the production of ATP is dependent on the amount of nitrate present in the reaction, and by then quantifying the ATP thus produced by measuring the light production of a luciferase.

In another preferred embodiment, the present invention provides a set of methods of detecting and/or quantifying the nitrate ion (NO₃⁻) by a method related to the method depicted in FIG. 1, but in which NAD⁺ is omitted from the reaction cocktail, nitrate reductase and NADH are added to the cocktail, and the concentration and/or quantity of NAD⁺ generated from NADH by nitrate reductase is monitored by measuring light production by luciferase. This reaction series is described in the scheme shown in FIG. 22. NO₃⁻ is an important contaminant of groundwater, especially from agricultural runoff; a potential marker for explosives; and possible biochemical marker for nictric oxide synthase (NOS) activity. This assay method may be applied to any or all of these needs. In this reaction series, pyruvate represents pyruvic acid, sodium pyruvate, potassium pyruvate, or another salt of pyruvate suitable for reaction with LDH, or a combination of the acid and such a salt. Pyruvate may also be supplied by an enzymatic reaction or series of enzymatic reactions, either within the same reaction vessel or in a prior reaction step. Similarly, lactate represents lactic acid, or a salt of lactic acid, or a combination of the acid and a salt. NAD⁺ and NADH represent the oxidized and reduced forms of nicotinamide adenine dinucleotide, respectively. G3P is glyceraldehyde-3-phosphate. Pᵢ is PO₄³⁻ or inorganic phosphate, supplied as sodium phosphate, phosphoric acid, or another suitable salt of phosphate. 1,3DPG is 1,3-diphosphoglycerate or 1,3-diphosphoglyceric acid. ADP is adenosine diphosphate. ATP is adenosine triphosphate. 3PG is 3-phosphoglycerate or 3-phosphoglyceric acid. hv is light. The enzymes employed are specified within boxes below the reactions they catalyze, respectively. G3PDH is glyceraldehyde-3-phosphate dehydrogenase. PGK is phosphoglycerokinase. Luciferase is firefly luciferase, beetle luciferase, or another ATP-dependent luciferase. In a reaction series intended to measure the quantity and/or activity of LDH, the reagents LDH, NAD⁺, and ATP are omitted or supplied in limiting quantities, while pyruvate, NADH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate. In a reaction series intended to measure the quantity of pyruvate, the reagents pyruvate, NAD⁺, and ATP are omitted or supplied in limiting quantities, while LDH, NADH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate. In a reaction series intended to measure the combined quantities of NAD⁺ and NADH, the reagents NAD⁺, NADH, and ATP are omitted or supplied in limiting quantities, while LDH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate.

In another aspect, the present invention provides a set of methods for measuring activity of the enzyme lactate dehydrogenase (LDH) by monitoring the concentration and/or quantity of NAD⁺ generated from NADH by the action of lactate dehydrogenase in reducing pyruvate to lactate, as shown in FIG. 21. The activity of LDH may be used to assess cell death or membrane damage, or it may be used as a marker or reporter enzyme, or for other purposes.

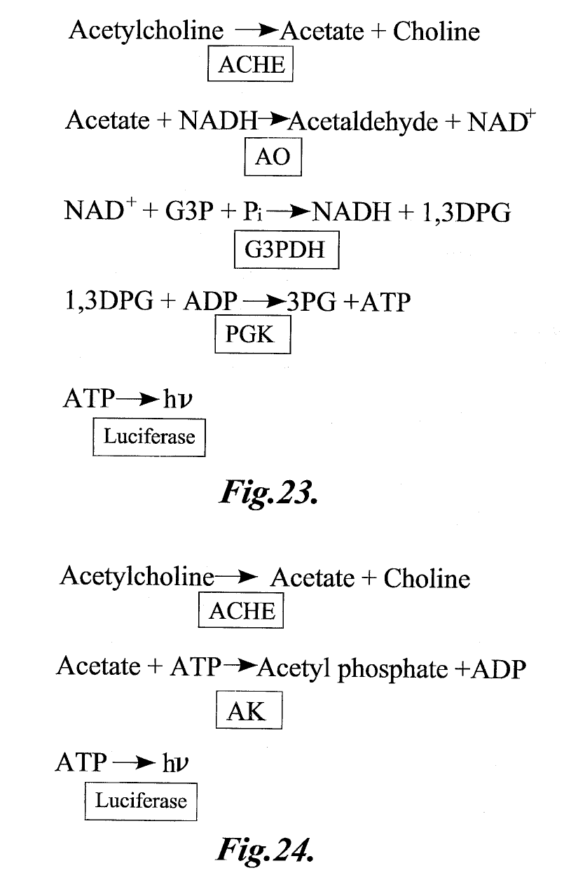

In another aspect, the present invention provides a set of methods for measuring activity of the enzyme acetylcholinesterase (ACHE) by monitoring the concentration and/or quantity of NAD⁺ generated from NADH by the action of an acetate reductase or aldehyde dehydrogenase or oxidase in reducing acetate to acetaldehyde, following cleavage of acetylcholine to acetate and choline, as shown in the scheme in FIG. 23. In FIG. 23, ACHE is the enzyme acetylcholinesterase, acetate represents acetic acid, sodium acetate, potassium acetate, or another salt of acetate suitable for reaction with LDH, or a combination of the acid and such a salt. Choline is a byproduct which plays no further role in these methods. AO is aldehyde oxidase, or any of many enzymes with NADH-dependent acetate reductase activity. NAD⁺ and NADH represent the oxidized and reduced forms of nicotinamide adenine dinucleotide, respectively. G3P is glyceraldehyde-3-phosphate. Pᵢ is PO₄³⁻ or inorganic phosphate, supplied as sodium phosphate, phosphoric acid, or another suitable salt of phosphate. 1,3DPG is 1,3-diphosphoglycerate or 1,3-diphosphoglyceric acid. ADP is adenosine diphosphate. ATP is adenosine triphosphate. 3PG is 3-phosphoglycerate or 3-phosphoglyceric acid. hv is light. The enzymes employed are specified within boxes below the reactions they catalyze, respectively. ACHE is acetylcholinesterase. AO is aldehyde oxidase, or an enzyme with NADH-dependent acetate oxidase activity. G3PDH is glyceraldehyde-3-phosphate dehydrogenase. PGK is phosphoglycerokinase. Luciferase is firefly luciferase, beetle luciferase, or another ATP-dependent luciferase. In a reaction series intended to measure the quantity and/or activity of ACHE, or determine the level of inhibition of ACHE, the reagents acetate, NAD⁺, and ATP are omitted or supplied in limiting quantities, while acetylcholine, NADH, G3P, Pᵢ, ADP, AO, G3PDH, PGK, luciferase, substrates and cofactors needed for the luciferase reaction, and other buffer constituents are supplied reagents, choline and 3PG are by-products that play no role, and 1,3DPG is a reaction intermediate. If the activity of ACHE is being measured, then ACHE is omitted from the cocktail and the light emission is related to the activity of ACHE in the sample. If inhibition of ACHE is being measured, or an inhibitor of ACHE is being detected, then ACHE is supplied, optionally in a fixed amount, and the light emission is inversely related to the degree of inhibition of ACHE. In a reaction series intended to measure the quantity of acetylcholine, the reagents acetylcholinesterase, acetate, NAD⁺, and ATP are omitted or supplied in limiting quantities, while ACHE, AO, NADH, G3P, Pᵢ, ADP, G3PDH, PGK, luciferase, substrates and cofactors needed for the luciferase reaction, and other buffer constituents are supplied reagents, choline and 3PG are by-products that play no role, and 1,3DPG is a reaction intermediate. In a reaction series intended to measure the quantity and/or concentration of acetate, ACHE and acetylcholine are unnecessary and are omitted, and the reagents acetate, NAD⁺, and ATP are omitted or supplied in limiting quantities, while AO, NADH, G3P, Pᵢ, ADP, G3PDH, PGK, luciferase, substrates and cofactors needed for the luciferase reaction, and other buffer constituents are supplied reagents, 3PG is a by-product that plays no role, and 1,3DPG is a reaction intermediate. Acetate may be supplied as the result of a separate enzymatic reaction or reaction series.

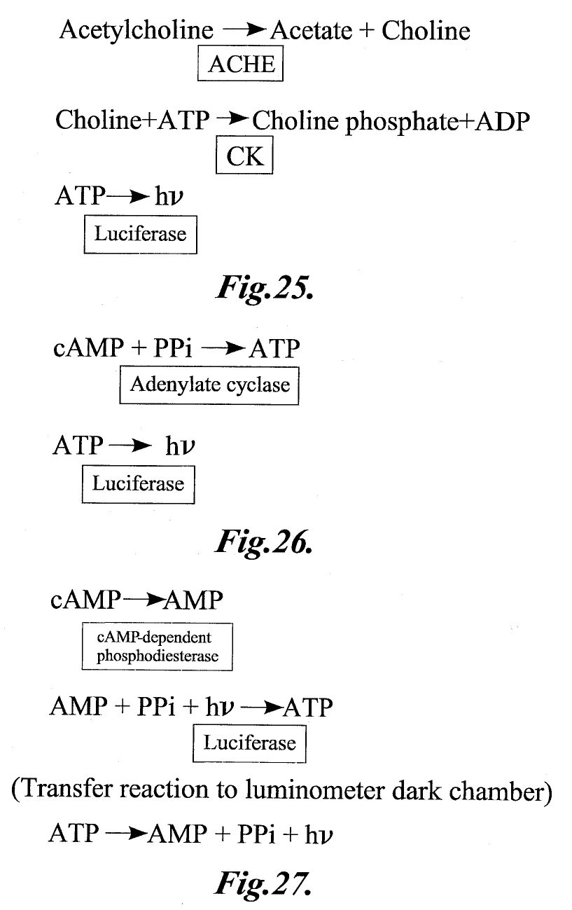

In another aspect, the present invention provides a set of methods for measuring activity of the enzyme acetylcholinesterase by a method that is independent of NAD⁺. Subsequent to production of acetate and choline by cleavage of acetylcholine catalyzed by acetylcholinesterase, either the enzyme acetate kinase or the enzyme choline kinase is used to phosphorylate acetate or choline, respectively, from the terminal phosphate group of ATP, and the reduction in ATP concentration is related to the activity of acetylcholinesterase (FIGS. 24 and 25). Here ACHE is the enzyme acetylcholinesterase. In FIG. 24, choline is a byproduct which plays no further role, and AK is an acetate kinase. In FIG. 25, acetate is a byproduct which plays no further role, and CK is a choline kinase. ADP is adenosine diphosphate. ATP is adenosine triphosphate. hv is light. The enzymes employed are specified within boxes below the reactions they catalyze, respectively. ACHE is acetylcholinesterase. AK is an acetate kinase. CK is a choline kinase. Luciferase is firefly luciferase, beetle luciferase, or another ATP-dependent luciferase. In a reaction series intended to measure the quantity and/or activity of ACHE, or determine the level of inhibition of ACHE, the reagent acetate (FIG. 24) or choline (FIG. 25) is omitted or supplied in limiting quantity, and ATP is supplied in a substantially determined quantity that may optionally be optimized by prior experiment for the particular application or anticipated degree of ACHE activity or inhibition, while acetylcholine, AK (FIG. 24) or CK (FIG. 25), luciferase, substrates and cofactors needed for the luciferase reaction, and other buffer constituents are supplied reagents, and choline (FIG. 24) or acetate (FIG. 25) and ADP are by-products that play no role. If the activity of ACHE is being measured, then ACHE is omitted from the cocktail and the light emission is inversely related to the activity of ACHE in the sample. If inhibition of ACHE is being measured, or an inhibitor of ACHE is being detected, then ACHE is supplied, optionally in a fixed amount, and the light emission is directly related to the degree of inhibition of ACHE. In a reaction series intended to measure the quantity of acetylcholine, the reagents acetylcholinesterase, acetate (FIG. 24) or choline (FIG. 25), and ATP are omitted or supplied in limiting quantities, while ACHE, AK (FIG. 24) or CK (FIG. 25), ATP, luciferase, substrates and cofactors needed for the luciferase reaction, and other buffer constituents are supplied reagents, and choline (FIG. 24) or acetate (FIG. 25) and ADP are by-products that play no role. In a reaction series based on FIG. 24 but intended to measure the quantity and/or concentration of acetate, ACHE, choline, and acetylcholine are unnecessary and are omitted, while AK, ATP, luciferase, substrates and cofactors needed for the luciferase reaction, and other buffer constituents are supplied reagents, and ADP is a by-product that plays no role. Acetate may be supplied as the result of a separate enzymatic reaction or reaction series. In a reaction series based on FIG. 24 but intended to measure the quantity and/or concentration of choline, ACHE, acetate, and acetylcholine are unnecessary and are omitted, while CK, ATP, luciferase, substrates and cofactors needed for the luciferase reaction, and other buffer constituents are supplied reagents, and ADP is a by-product that plays no role. Choline may be supplied as the result of a separate enzymatic reaction or reaction series.

These methods, which yield a negative light signal (i.e., the amount of light generated is reduced by the action of the test enzyme, acetylcholinesterase, or one or more of the test reagents acetate, choline, or acetylcholine), has important potential advantages in specific applications over a method with a positive signal. In a military, bioterrorism, or environmental setting, if an inhibitor of acetylcholinesterase is present, such as nerve gas or certain pesticides, light will be present, due to the failure of acetylcholinesterase to produce acetate or choline, and the consequent inability of the respective kinase to degrade ATP. Thus danger is represented by a positive light signal. Conversely, if the dangerous agent is not present, acetylcholinesterase will produce the kinase substrate, and the ATP will be consumed. The presence of a positive light signal for danger, and its absence representing safety, may be more acceptable to the operator than relying on the absence of a light signal to indicate danger. This reaction series and the above NAD⁺-dependent reaction series may be adapted to military use, or use in civilian anti-terrorism applications. Among the possibilities are adaptation of the methods to detection of acetylcholinesterase inhibitors in an air-collection device for clearance of a threat area in military operations, adaptation to a water-sampling device for military or environmental purposes, development of a stand-off air-sampling or water-sampling device for safe military and civilian sampling of highly hazardous areas, and development of a continuous-flow device for monitoring of air or water. Such a device could be connected to an alert or alarm mechanism if a concentration of an acetylcholinesterase inhibitor higher than a threshold value were detected.

In another aspect, the present invention provides a general set of methods for measuring activity of the enzyme acetylcholinesterase by coupling the presence of a product of acetylcholinesterase catalysis (which may be acetate, choline, or acetylcholine, the latter if acetylcholinesterase is working in the so-called “reverse” or synthetic direction) to production of ATP by means of an enzymatic reaction or series of enzymatic reactions, such that the amount of ATP produced is dependent on the activity of acetylcholinesterase, and by then quantifying the ATP thus produced by measuring the light production of a luciferase.

It will be evident to one skilled in the art that the various assays described herein may be adapted to the form of a kit; a portable or fixed microfluidics device; a single-use or multiple-use disposable device; a diagnostic assay for clinical use, home use, or use in a doctor's office; or a flow cell with continuous or long-term operation. A flow cell incorporating elements of the reaction series described could involve immobilized enzymes, such that a continuous reading is obtained.

It will be evident to one skilled in the art that the various assays described herein may be adapted to a format in which all reagents needed for analyte-dependent generation of light may be combined in a single reagent mixture (a “one-step reaction”). However, the reagents and processes may also be divided into multiple reactions. Such a strategy might be desirable, for example, if a given sample were kept at a different pH, such that one or more enzymatic reactions would be carried out at a pH that is different from that of the light-generation reaction or reactions; the sample resulting from this preliminary reaction could then be transferred or aliquoted into the light-generation reaction or reactions. Another example is a reaction that requires high temperatures. Such a reaction could be carried out in the absence of the light-generating reaction or reactions, followed by addition of the light-generating reagents and performance of one or more steps at lower temperatures. In general, reactions that require or occur preferentially under conditions that are incompatible with the light-generating reaction or reactions may be carried out under those conditions, whereupon all or part of the resultant sample may be transferred or aliquoted to a separate reaction vessel, where the light-generating reaction or reactions may be carried out.

In the examples described further below, assays with specific parameters are exemplified. However, the present invention provides a set of methods and compositions for coupled luminescent assays using various concentration ranges of the chemical and biochemical components specified for the methods described in EXAMPLES 18, 19, 20, and 22 herein, such that the assays function with the following concentrations:
Dithiothreitol (DTT): 0.00001 to 1M final concentration;
Adenosine diphosphate (ADP): 10⁻¹⁰ to 10⁻² M final concentration, or alternatively, ultrapurified ADP: 10⁻¹⁰ to 10⁻² M final concentration;
Nicotinamide adenine dinucleotide, oxidized form (NAD⁺): 10⁻¹⁵ to 10⁻¹ M final concentration, or zero to 10⁻¹ M starting concentration before initiation;
Glyceraldehyde-3-phosphate: 10 nM-100 mM final concentration;
Triethanolamine: 0-1M final concentration;
Sodium phosphate: 0.01 mM to 1 M final concentration;
Ethylamine diamine tetraacetic acid: 0-5 mM final concentration, or higher if appropriate multivalent cations are present;
Bovine serum albumin: 0-20 mg/mL final concentration;
ATP assay cocktail: 1-85% final concentration;
ATP assay diluent: 0-90% final concentration;
Phosphoglycerokinase (PGK): 1 in 10¹¹ parts to 1 in 10³ parts final concentration (assuming stock solution at approximately 5000 units per mL), or alternatively, ultrapurified PGK: 1 in 10¹¹ parts to 1 in 10² parts final concentration (assuming stock solution at approximately 5000 units per mL);
Glyceraldehyde-3-phosphate dehydrogenase: 1 in 10¹¹ parts to 1 in 10 parts final concentration (assuming stock solution at approximately 4300 units per mL);
IMDM—0-80% of the final reaction volume;
PBS—0-80% of the final reaction volume.

Moreover, in certain specific examples below, in combination with the concentration ranges listed above, the following concentrations may be used.

In EXAMPLE 19:
Tris-buffered saline, pH 7.4—zero to 99.9% of final volume;
Pyruvate salts and/or pyruvic acid, 10⁻¹⁰ to 10⁻¹ M final concentration;
LDH, 0 to 10¹⁰ units per liter, as a limiting reagent provided in the sample or standard.

In EXAMPLE 22:
Nitrate reductase, 10⁻¹² to 10⁹ units/liter final concentration, or, if nitrate reductase is a test enzyme, 0 to 10⁹ units/liter final concentration;
MOPS: 0-3 M final concentration;
Glycerol: 0-50% final concentration;
Tomato Plant Food or another nitrate source, supplying nitrate in the test sample and/or as a standard and/or in the cocktail for assay of a separate enzymatic activity: 0-1 M final concentration of nitrate.

Moreover, in EXAMPLE 21, an assay with specific parameters is exemplified. However, the present invention provides a set of methods and compositions for coupled luminescent assays using various concentration ranges of the chemical and biochemical components specified for the methods described in EXAMPLE 21, such that the assay functions with the following concentrations:
Acetylcholine: 10⁻¹⁸ M to 1 M final concentration;
ATP Assay Diluent: 0-90% of final reaction volume;
ATP Assay Mix: 0-85% of final reaction volume;
ATP: 10⁻¹² M to 1 M final concentration;
PBS: 0-80% of final reaction volume;
Acetate kinase: 10⁻¹⁵-10⁹ units per liter final concentration;
Triethanolamine: 0-1 M final concentration;
Tris: 0-3 M final concentration;
Acetylcholinesterase, measured in sample or supplied in cocktail or as standard: 0-10¹² units/liter final concentration.

The following examples are offered by way of illustration and not by way of limitation.

Chemicals and biochemicals were purchased from Sigma-Aldrich (St. Louis, Mo.). Growth medium (IMDM) was purchased from Irvine Scientific Corporation (Santa Ana, Calif.).

### Example 1

### Measurement of a Cytolytic Process by DeathTRAK

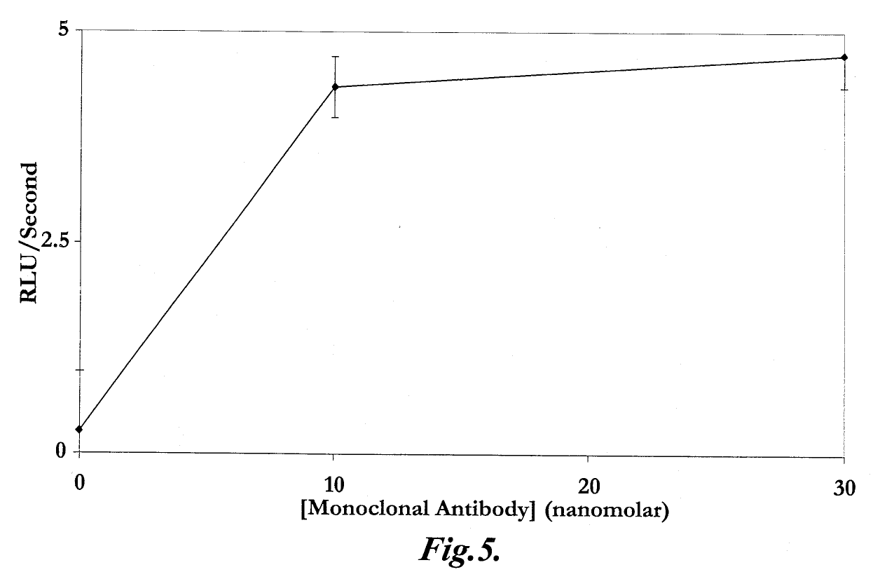

The unoptimized DeathTRAK assay cocktail was used to measure the effect of an anti-Factor I antibody on complement-mediated lysis of the PC-3 prostate-cancer cell line. Cells were grown in Iscove's Modified Dulbecco's Medium (IMDM) with 10% fetal bovine serum, then treated with 0.25% trypsin/EDTA to allow removal from the growth flask, and subsequently washed with IMDM to remove trypsin and EDTA. Assays were performed in triplicate. Since complement requires some time to act against its target, the cells were incubated with complement serum and other components (see composition below) for 100 minutes at 37° C. in a covered Costar low-binding plate (Cat. #3596) before the data in FIG. 5 were taken. A 0.00.5-mL aliquot of each complement reaction was then transferred to wells of a microtiter plate, along with a control using complement that had been heat-inactivated at 60° C. for two hours. Because the DeathTRAK cocktail is compatible with live cells, it was not necessary to remove the cells or otherwise treat the reaction mixture prior to the cytotoxicity assay. The microtiter plate was transferred to the luminometer. Each well was injected with 0.045 mL of reaction cocktail (composition below). The no-complement rate has been subtracted from each data-point on both charts. The averages of three runs are shown.

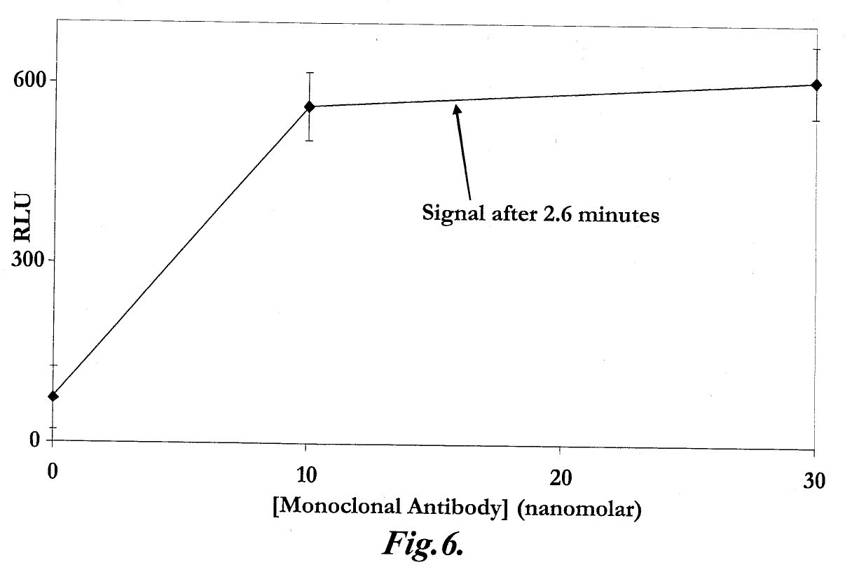

Several conclusions are evident from the data. First, in spite of the difference in the scales and the fact that FIG. 5 reports a rate of change of luminance, while FIG. 6 reports an absolute luminance, the two figures appear to show almost identical phenomena. This means that almost all of the accuracy and information of obtaining linear fits from the 20-minute run is captured in a 2.6-minute run with a single readout. This is a general phenomenon, and single-point readouts after an interval of 1-3 minutes are very useful, as long as automated injection is used. However, if the reagent cocktail is loaded manually, then the interval between the initiation and the luminance read will not be the same for each sample, and absolute luminance readouts will not be useful. Instead, the user would take a linear fit, typically of the data from approximately 1-3 minutes of the reaction. This yields a rate of increase of the luminance signal, which eliminates the effect of the variations in intervals between initiation and readout among the samples. In general, in the high-throughput setting, the reagent cocktail is injected automatically, and single-point reads will yield excellent results. The standard deviations of the triplicate runs are small in each case and are actually smaller in the single-point data. Finally, both runs show the anticipated effects, including the fact that complement alone has a small effect without antibody (zero-antibody point), but the antibody greatly enhances complement-mediated killing at 10-30 nM.

TABLE I
Complement Lysis Mixture Composition

| Material | Percentage |
|---|---|
| IMDM | 9.6% |
| Mg-EGTA | 3% |
| Human complement serum | 40% |
| PC-3 cells (100,000/mL) in IMDM | 45% |
| PBS, with or without anti-Factor I monoclonal R65 | 2.4% |

The Mg-EGTA component was made up as follows:

300 mM magnesium chloride and 200 mM ethylene glycol tetraacetic acid in H₂O, brought to pH 7.5 with NaOH and pass through a 0.22-micron filter.

0.005 mL of the lysis reactions was removed and mixed with 0.045 mL unoptimized DeathTRAK cocktail (0.25 mL 4×GP cocktail, 0.125 mL ATP assay reagent, 1.125 mL ATP assay diluent, 2.3 mL IMDM containing 10% fetal bovine serum, 0.0025 mL 1:1,000,000 PGK in PGK diluent). Thus this assay was not performed in homogeneous mode. Example 2, below, explains how the reagent cocktail was formulated and optimized for homogeneous mode. The wells were read for luminance for 1 second immediately after injection, and subsequently every 150 seconds for a total of 20.1 minutes (1205 seconds). The timepoints from 305 to 1205 seconds were taken for data reduction by linear regression, using Microsoft Excel (FIG. 5). In addition, single timepoints after 155 seconds of incubation were taken as endpoints for comparison (FIG. 6).

The 4×GP cocktail was made as follows:
10 mL 5×PGK diluent
0.05 mL 1M DTT
0.00295 mL 100 mM ADP
0.5 mL 100 mM NAD+
0.52 mL glyceraldehyde-3-phosphate (49 mg/mL as purchased)
1.425 mL dH₂O

The 5×PGK diluent was made as follows:
3.73 g Triethanolamine (TEA)=25 mmol
1.5 g NaH₂PO₄
1.295 mL 193 mM Ethylene Diamine Tetraacetic Acid (EDTA) pH 8.0
25 mg Bovine Serum Albumin (BSA) Fraction V
Titrated to Ph 7.0 with concentrated HCl and made up to a final volume of 50 mL.

PGK diluent (1×) was made up by diluting one part of 5×PGK diluent with four parts deionized H₂O.

### Example 2

### Improved Measurement of G3PDH Activity and/or Cytolysis and/or Membrane Damage by Optimized DeathTRAK

This Example demonstrates the process of developing the method into a homogeneous assay suitable for use in high-throughput screening. This includes: Example 2A, in which the cocktail is optimized for signal strength while maintaining compatibility with live cells; EXAMPLES 2B and 2C, in which the PGK and ADP concentrations, respectively, are optimized; EXAMPLE 2D, in which the optimized cocktail is tested for linearity and dynamic range; EXAMPLES 2E and 2F, in which the storage conditions are tested and optimized; EXAMPLE 2G, which shows the advantages of protecting the DeathTRAK cocktail from light or adding the PGK component shortly before reaction initiation; and EXAMPLE 2H, in which the use of a stop reagent is demonstrated.

### Example 2A

### Titration for Optimum Ratio of IMDM to PBS at Low Signal Strength

In this Example, the concentrations of PBS and IMDM, both of which are cell-compatible buffers, were varied inversely in order to determine the optimum composition for cell compatibility and high signal strength. A cocktail was made consisting of 0.114 mL 4×GP cocktail, 0.057 mL ATP assay cocktail, 0.513 mL ATP assay diluent, 0.0011 mL 1:1,000,000 PGK, and 0.00057 mL DTT. 0.0229 mL of this cocktail was distributed to each of 24 wells of a luminescent microtiter plate. 16 wells also received 0.005 mL of 1:100,000-diluted G3PDH, while the other 8 wells received only 0.005 mL G3PDH dilution buffer. The 16+enzyme wells and the eight-enzyme wells then received amounts of IMDM and PBS varying from 0-100% of the 0.0221 mL remaining in the 0.05-mL reaction. The plate was then read for luminance for one second each 60 seconds for 10 minutes. The last seven timepoints were analyzed by linear regression and the duplicate rates (+enzyme only) were averaged. The results showed a broad maximum in activity from 60-100% IMDM and 40-0% PBS (the final concentration range after addition of the other cocktail components and the sample was 26.5-44.2% IMDM) and 17.7-0% PBS). Any concentration ratio in this range may he used.

Composition of the G3PDH dilution buffer was:
1000 parts PGK diluent
1 part 1M dithiothreitol

### Example 2B

### Optimization of PGK Concentration

The assay suffered from poor linearity, especially with [G3PDH]. It was hypothesized that a deficit of phosphoglycerokinase (PGK) was causing this problem. There are both upper and lower constraints on the concentration of this enzyme, because the commercial preparation typically comes with some contaminating G3PDH, which causes dynamic background. The following experiment was used to optimize the PGK concentration for use in the rapid, homogeneous format.

0.483 mL IMDM

0.2535 mL PBS

0.127 mL 4×GP cocktail

0.0633 mL ATP assay cocktail

0.5703 mL ATP assay diluent

0.0006 mL 1M DTT

0.2488 mL of this cocktail was aliquoted into each of five reaction vessels, which received 0.00125 mL of varying dilutions of PGK:

| Vessel | 1 | 2 | 3 | 4 | 5 |
|---|---|---|---|---|---|
| PGK | 3 × 10⁻⁶ | 1 × 10⁻⁵ | 3 × 10⁻⁵ | 1 × 10⁻⁴ | 3 × 10⁻⁴ |
| Dilution | | | | | |

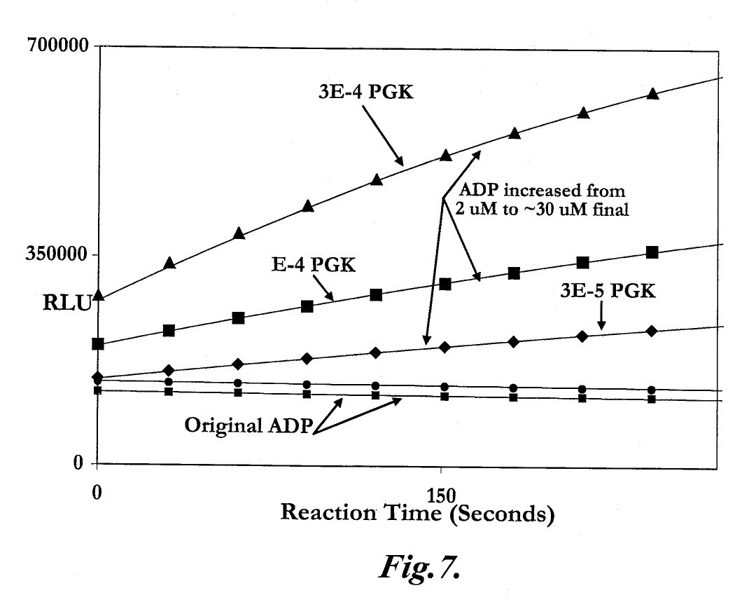

The contents of each reaction vessel were aliquoted in duplicate onto a microtiter plate. A fixed amount of 0.005 mL of 1:10,000-diluted G3PDH was added in duplicate to each PGK dilution and the reactions were read for luminance. The results showed that PGK diluted 1×10⁻⁴ from the purchased reagent yielded an excellent signal, although saturation was seen, which proved to be due to exhaustion of ADP. Still higher concentrations of PGK led to sublinear behavior even after the ADP concentration was adjusted (see EXAMPLE 2C, FIG. 7). This concentration of PGK (1×10⁻⁴) was therefore selected for the optimized cocktail. However, since the level of G3PDH contamination in a different lot of PGK could be higher, it may be necessary to test each lot for this problem when in commercial production. If the G3PDH contamination is unacceptably high, another source can be found, or the PGK enzyme can be purified away from G3PDH, or labile, irreversible inhibitors of G3PDH such as iodoacetic acid can be used to inactivate the contaminant.

### Example 2C

### Adjustment of ADP Concentration

The saturation seen after PGK optimization was likely to be due to exhaustion of a consumable component from the reaction. ADP was a candidate component because the concentration of ADP that can be used is limited by the fact that commercial ADP preparations are contaminated with ATP, which increases the static background.

In this experiment, ADP was increased from 2 μM (original) to 30 μM after the reactions described in Example 2B had been running for 2.4 hours, and luminance was read. ADP was clearly limiting in all three reactions, and the addition of ADP to the reactions with optimized PGK led to a rate of over 1600 RLU/sec with almost no loss in linearity (R2>0.999) over the first 150 seconds (FIG. 7). Before addition of ADP, the reactions were reaching saturation at ˜160,000 RLU, but in the experiment depicted in FIG. 7, there was little deviation from linearity, even at 600,000 RLU. The composition of the new optimized cocktail was (2.9974 mL final volume):

TABLE II
Composition of Optimized DeathTRAK Cockail

| | Amount (for 2.9974 mL |
|---|---|
| Material | Final Volume) |
| IMDM | 0.936 mL |
| PBS | 0.507 mL |
| 10⁻⁴-diluted PGK | 0.0025 mL |
| 4XGP cocktail | 0.2535 mL |
| ATP assay cocktail (freshly dissolved) | 0.1266 mL |
| ATP assay diluent | 1.1406 mL |
| 1M dithiothreitol | 0.0012 mL |
| 2.8 mM adenosine diphosphate | 0.03 mL |

### Example 2D

### Tests of the Optimized Cocktail

The optimized cocktail was also tested with the G3PDH test enzyme by dilution over two orders of magnitude, yielding a linear correlation of >0.9998, with coefficients of variation of individual points ranging from 3-6%.

The optimized cocktail was also tested against another method of determining cytotoxicity. FIG. 3 shows a comparison of DeathTRAK results with an independent, “blinded” estimate of killing by a cell-culture technician visualizing the cells through a microscope. The close correlation of 0.990 demonstrates, first, that the maximum DeathTRAK signal corresponds to death of all the cells, and second, that the DeathTRAK method agrees well with another technique. Of course direct visualization is too labor-intensive to use on a regular basis. The methods used were the same as those used for the 841CON and PC-3 cell lines in the experiments reported under Example 8, except that the total-lysis step was not performed.

The optimized DeathTRAK cocktail was also used to test sensitivity and linear response to dead Raji cells. Non-adherent Raji cells were harvested, resuspended at the same concentration (300,000/mL) in Lysis Buffer B (Lys.B, Phosphate buffered saline plus 1% Nonidet P-40), and incubated for 10 minutes at room temperature to kill them. They were then serially diluted with Lys.B to yield the indicated numbers of cell equivalents per mL. 0.005 mL of each dilution in triplicate was mixed with 0.045 mL of optimized DeathTRAK cocktail and luminance was read for 1 second every 60 seconds for ˜720 seconds. The dose-response curve of the assay over four orders of magnitude is shown in FIG. 4.

### Example 2E

### Optimization of Storage Conditions

### Full and Partial Cocktails, Lyophilization Vs. Freezing

In this experiment, the unoptimized cocktail (see EXAMPLE 1) was made up with or without various components and frozen at −80° C. or lyophilized; the aliquots were then thawed or reconstituted and tested with PC-3 cells. Tube 1 contained the full cocktail; tube 2 contained everything except PGK; tube 3 contained everything except PGK, ATP assay cocktail, and ATP assay diluent; tube 4 contained the 4×GP cocktail only. Each of these 4 tubes contained enough constituents to make up 3 mL final of the cocktail. The contents of each of the 4 tubes were aliquoted into 5 storage tubes each (containing enough constituents for 0.5 mL final of the cocktail). The 5 tubes of each set were treated as follows: aliquot 1 was lyophilized and stored frozen (−20° C.) for 1 day; aliquot 2 was lyophilized and stored at room temperature for 1 day; aliquot 3 was lyophilized and stored frozen (−20° C.) for 4 days; aliquot 4 was frozen immediately (−80° C.) and stored for 1 day; aliquot 5 was frozen immediately (−80° C.) and stored for 4 days. Because the final cocktail is approximately 33.8 mM in TEA, this amount of TEA was added to the lyophilized aliquots for reconstitution.

The aliquots were tested in duplicate (0.045 mL each) with 0.005 mL of PC-3 cells killed by diluting 1:100 into Lys.B. (final 3000 cells/mL). Linear fits were taken of the luminance reaction. After 4 day's storage, room temperature had completely killed the reactions with ATP assay cocktail present and destroyed most of the activity even with the ATP assay cocktail stored separately. Lyophilization was also clearly inferior to freezing. Subsequently tests with the optimized cocktail showed that the best and most convenient storage method was to make the non-labile cocktail described below under Example 8 and store it separately at −20 C or −80 C, adding the ADP (stored at −20 C or −80 C), PGK (stored at +4 C), and ATP Assay (stored at −20 C or −80 C) components either on the day of use or immediately before use.

### Example 2F

### Stability of the Full Cocktail

### Effects on Static Background of Freezing Vs. 4° C.

In formulating a homogeneous assay it was necessary to determine not only how well the assay activity would survive storage, but also how the static background would he affected (the dynamic background is due to enzyme activity and would not be expected to increase upon storage). The full non-optimized cocktail with or without PGK present was subjected to storage at 4° C. or −80° C. The aliquots were then checked with 0.005 mL Lys.B (containing no cells) for initial luminance value. Storage of the cocktail with or without PGK present made essentially no difference, but storage at −80° C. caused an increase of <30% in the static background, compared to an increase of >1000% at 4° C. This confirmed the benefits of freezing the cocktail components (other than PGK), as mentioned under EXAMPLE 2E.

### Example 2G

### Elimination of Lag Phase by Protecting Cocktail from Light after Addition of PGK

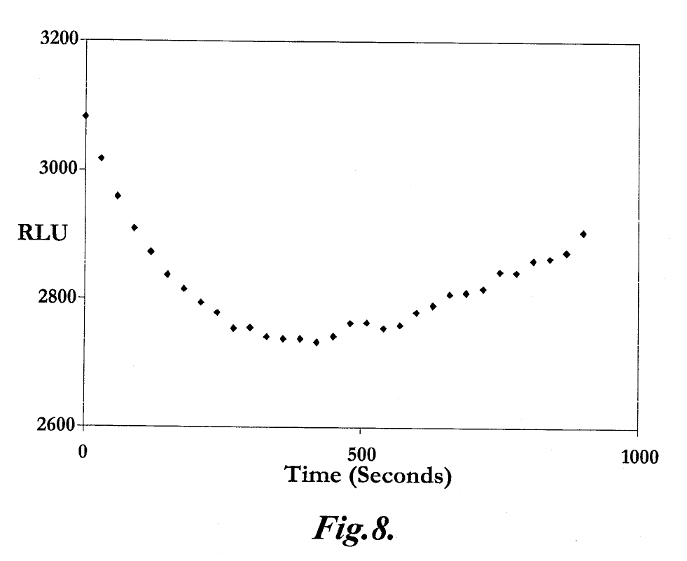

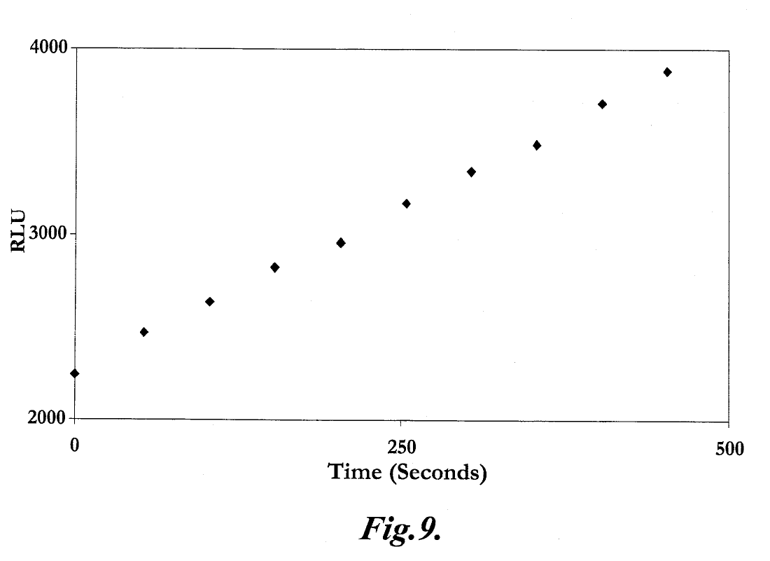

FIG. 8 shows an example of a reaction in which the cocktail was not protected from light for a substantial period of time after addition of PCTK. If the assay is run within a few minutes after addition of PGK, the lag phase is not seen, but if a significant amount of time elapses after addition of PGK., then the small amount of G3PDH enzyme contaminating the PGK preparation causes a slow accumulation of ATP. This ATP reacts with luciferase to generate light, adenosine monophosphate (AMP), and inorganic pyrophosphate (PPᵢ), but in the presence of light, the backward luciferase reaction is also possible, i.e., AMP, PPᵢ, and light can be combined by luciferase to make ATP. Because of these reactions, a steady-state level of ATP is achieved. When the plate is then transferred to the interior of the luminometer, which is completely dark, the backward reaction becomes impossible. As a result, the extra ATP present is rapidly broken down by luciferase, leading to a rapidly declining signal during the first 5-10 minutes of the reaction. Eventually, the extra ATP is exhausted, and the normal, linear signal due to the G3PDH in the test sample is revealed. To avoid this problem, the user needs to prevent the ATP level in the cocktail from rising. This is accomplished either by withholding the PGK component until shortly before the reaction is initiated, or by protecting the cocktail from light. The latter method is more suitable for a high-throughput screening environment, in which a timed addition of a reagent to the cocktail prior to each run is inconvenient. Under these circumstances the reagents can be mixed and kept in an opaque or dark-glass bottle. Even if the cocktail was exposed to light during the process, the steady-state level of ATP will decline to an acceptable value after the cocktail is shielded from light. FIG. 9 shows the results of a run in which the cocktail was protected from light after addition of PGK. There are no substantial deviations from linearity in the run.

### Example 2H

### Use of a Stop Reagent

To demonstrate the use of a stop reagent, DeathTRAK reaction cocktail was made as for EXAMPLE 1. To a 0.5-mL aliquot of this cocktail, 0.0016 mL of 1:1,000,000-diluted PGK were added (termed “+PGK” cocktail). Two 0.045-mL aliquots of the standard unoptimized cocktail and two 0.045-mL aliquots of the “+PGK” cocktail were measured into a luminescent microtiter plate. 0.005 mL of 1:100,000-diluted G3PDH was transferred to each of these four aliquots of cocktail. After 21 minutes' incubation at room temperature, 0.02 mL of 20 mM bromopyruvic acid (BPV) dissolved in ATP assay diluent was added to one reaction with the unoptimized cocktail and one reaction with the “+PGK” cocktail 0.020 mL, of ATP assay diluent alone was added to the other two reactions. This quantity of BPV (˜3 mM final) stopped the increase in luminance in the reactions both with and without added PGK. In fact there is a small negative rate of change of luminance in the stopped reactions, but this is likely to be due merely to exhaustion of ATP by luciferase. Use of this or an alternative stop reagent allows the user to delay reading the plate, while maintaining the relative signal strengths of the samples.

### Example 3

### Software for Analysis of DeathTRAK Data

An Excel macro was written which seeks the best linear fit of four or more consecutive timepoints for each well and reports the rate calculated from the fit, the correlation coefficient, and the identity of the time range that yielded the best fit. Currently the macro also outputs all of the fit rates and correlation coefficients onto the spreadsheet, but this could easily be switched off. At present the macro has a limitation of 10 timepoints for each well, but it would be evident to one skilled in the art that this can be increased.

### Example 4

### Measurement of Bacteriolysis by DeathTRAK

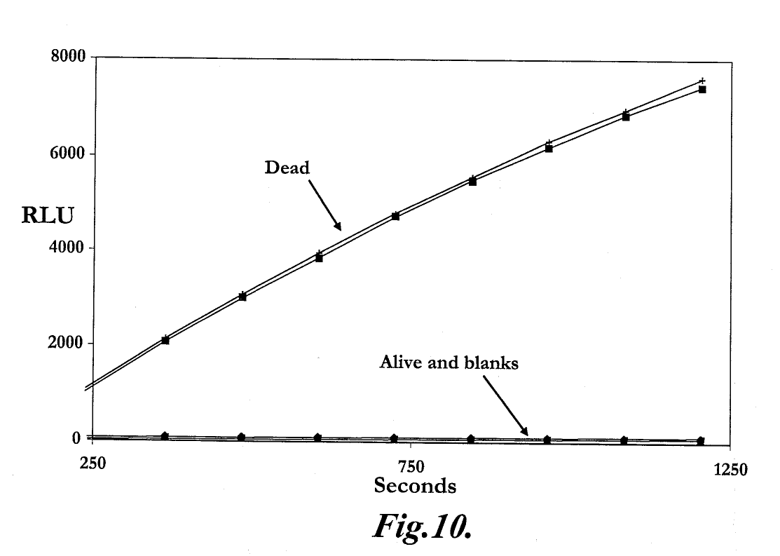

E. coli strain EV-5 was lysed by resuspension in Somatic Cell ATP Releasing Reagent (SCARR, from Sigma-Aldrich). Bacteria grown overnight in LB were washed twice with PBS and resuspended in the same volume of PBS. Cells were then diluted 1:100 into SCARR and incubated for ten minutes at room temperature. Cells were either used from this mixture or further diluted 1:100 into PBS. Live cells were diluted directly into PBS. The quantity of dead cells was measured by adding 0.005 mL of the suspension or a 1:100 dilution of the suspension in PBS to 0.045 mL reaction cocktail (composition below) and reading the luminance for two seconds every two minutes for 20 minutes. FIG. 10 shows progress curves taken with duplicate 1:100 dilutions of the dead EV-5 cells vs. the same dilutions of live cells and blanks with no cells. The signal associated with the dead cells is very strong, linear, and highly reproducible (both runs are shown). Live cells gave a very faint signal: a 1:10,000 dilution of dead cells gives a very similar signal to a 1:100 dilution of live cells, indicating that leakage from live cells yields about 1% of the signal of dead cells.

Composition of Reaction Cocktail:
0.09 mL 4×GP
0.0009 mL 1:1,000,000-diluted PGK
0.07 mL PBS
0.3325 mL ATP assay diluent
0.0175 mL ATP assay cocktail

### Example 5

### Aldolase-DeathTRAK

In an alternative embodiment of the invention, the G3P component is omitted from the cocktail and two reagents are substituted fructose-1,6-bisphosphate (FBP), and aldolase (the enzyme which cleaves FBP to G3P, which is converted by G3PDH to the substrate for PGJK, and dihydroxyacetone phosphate, which plays no role). The formulation of the Aldolase-DeathTRAK cocktail is as follows (5 mL)
0.609 mL 2 mg/mL FBP
0.05 mL 0.1 M DTT
0.00077 mL 38.39 mL ADP
0.005 mL 1:100,000-diluted PGK
0.004 mL 1:1000-diluted aldolase (in PGK diluent)
0.5 mL 10×PGK diluent
3.83 mL dH₂O

E. coli strain EV-5 at A₆₀₀ of 0.703 were diluted 1:10 into LB and washed 3× with an equal volume of PBS. Cells were lysed by complement in a reaction of the same composition as that used in EXAMPLE 1 except that PBS was used instead of IMDM, 0.15 mL of cells were added to 0.15 mL of the reactions containing either active complement (run in quadruplicate) or complement that had been inactivated at 60° C. for two hours (in duplicate). Lysis was measured by removing 0.03 mL, of the lysis reaction, centrifuging for 3 minutes at ˜1500×g, and transferring 0.01 mL of the supernatant to a luminescent microtiter plate. A mixture of 0.04 mL Aldolase-DeathTRAK cocktail (above) and 0.15 mL ATP assay cocktail diluted 1:20 into ATP assay diluent was then added to each sample. The luminance was read after 23 minutes. Results of duplicate reactions were: +complement, 1.793±0.173 RLU/Sec; −complement, −0.229±0.037 RLU/Sec (p<0.004). The Aldolase-DeathTRAK reaction easily distinguished the effects of active from inactive complement against the E. coli cells.

### Example 6

### Use of DeathTRAK or Another Coupled Luminescent Assay to Measure Effects of a Cytotoxic or Membrane-Damaging Entity

The use of DeathTRAK, or another coupled luminescent assay as described below under EXAMPLE 14, to measure the cytotoxicity of a compound or drug candidate would be similar to its use with complement (EXAMPLE 1). The DeathTRAK cocktail may be introduced before, during, or after the potentially cytotoxic agent was mixed with the cells, depending on the kind of test being performed. If a quantitative estimate of killing rate were desired, the cells could be mixed with the potentially cytotoxic agent first and incubated for a fixed interval, after which the DeathTRAK cocktail would be added; this would provide an accurate picture of aggregate cell death over time. For maximum speed, DeathTRAK, cells, and the potentially cytotoxic agent could be mixed simultaneously; depending on the speed of killing, a signal could be obtained within minutes, or possibly even less than one minute Finally, mixing DeathTRAK with cells before addition of the potentially cytotoxic agent would allow comparison of the viability before and after treatment. These last two modes would also allow the user to follow the whole toxicity reaction in real time. A calibration standard of cells could be used to obtain absolute quantification.

### Example 7

### Measurement of Cell Proliferation

For a number of uses, it is preferable to measure live rather than dead cells. By doing this, the user can measure effects such as cytostatic and growth-inhibitory behavior, in addition to cytotoxicity. This is often done either with a viability assay that directly measures metabolism (such as Alamar Blue, MTT, or WST) or a method that involves killing all the cells and immediately measuring release of a substance (usually ATP). The problem with the first type of method is that viability assays do not measure the number of live cells at an instant in time, but rather an integral of metabolism over an interval. Also, some of the reagents (such as MTT) have been shown to interfere with metabolism, and/or to be sensitive to redox-active chemicals such as antioxidants. The ATP-release method is destructive but is quite rapid and sensitive. DeathTRAK or another coupled luminescent assay as described under Example 14 is also useful in this mode. The DeathTRAK cocktail may again be added before, after, or simultaneously with the lytic reagent. The luminance readout after lysis and addition of DeathTRAK would correspond with the total cell number. A calibration standard could be used as under EXAMPLE 6. Examples of lytic agents for use with various cell types are provided in EXAMPLE 8. The same lytic agents would be useful if the user desires only a proliferation/viability readout. G3PDH, the enzyme which provides the DeathTRAK readout, is not subject to the same types of metabolic fluctuations as ATP; thus the viability readout of DeathTRAK will often be more closely correlated with cell number than that of the ATP-release assay. A further advantage of DeathTRAK and other methods of the present invention in this mode is that it allows a continuous readout, so that the user can decide to allow the signal to increase further for a later read if desired sensitivity has not yet been achieved. This is not possible with the ATP-release assay.

### Example 8

### Combined Cytotoxicity/Proliferation Mode

In the preferred mode of DeathTRAK use, this Example shows how the information available under both EXAMPLES 6 and 7 may be gathered in a single experiment. DeathTRAK or another coupled luminescent assay as described below under EXAMPLE 14 can be used to measure both live and dead cells in a single reaction vessel.

The cocktail is added to the cytotoxicity reaction before lysis and the luminance rate (or a single timepoint) is measured; this represents cells killed by the process under test. A lytic agent compatible with DeathTRAK activity is then added. The rate (or single timepoint) observed after lysis represents the total cell number present (live plus dead). To obtain the number of live cells present before lysis, the signal before lysis is subtracted from the signal after lysis. A calibration standard of cells can be used as under EXAMPLE 6.

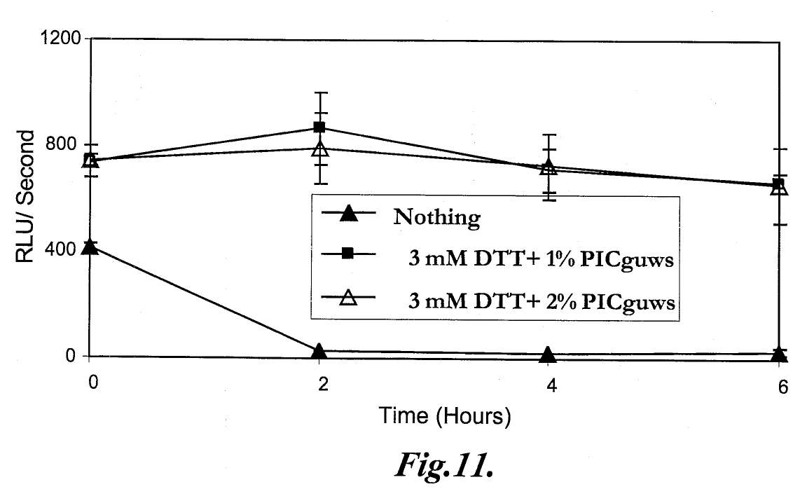

If the user wishes to measure the cytotoxic effects of a given compound, mixture, or biological entity, it may be desirable to incubate the target cells with the potential toxin prior to performance of the assay. While DeathTRAK itself is very rapid, in some cases toxic effects require some time to result in increased release of cellular contents, and/or reductions in cell viability and/or proliferation. During this time, it is possible for some of the possible release enzymes, such as G3PDH in the case of DeathTRAK, to be altered or attacked by the cell environment, or by the aerobic medium in which the cells are growing. FIG. 11 shows that it is possible to protect G3PDH from most or all of the effects of the cellular/growth medium environment by using a judicious mixture of protective agents. In this case the protective agents were 3 mM (final) dithiothreitol, as a reductant, and 1% PICguws, a protease inhibitor cocktail available from Sigma-Aldrich as catalog number P-2714. In these experiments, PC-3 cells were grown to near confluence, trypsinized to resuspend them, and diluted to 20,000 cells/mL in IMDM, with or without 3 mM dithiothreitol and 1% or 2% PICguws. 50-μL aliquots of this cell mixture were transferred to a luminescent microtiter plate and incubated for the lengths of time indicated in FIG. 11. At the timepoints, 45 μL of DeathTRAK cocktail was added to triplicate wells and the luminance was measured. Without the protective reagents the G3PDH activity rapidly declines to near zero, but the protective combination leads to very little loss in activity over five hours. In certain cases, one or both of the components of this protective cocktail may be found to interfere with the activity of one or more molecules under test, in which case (1) the protective cocktail may be adjusted or changed, (2) the interference may be measured and accounted for, (3) the length of the incubation prior to addition of the DeathTRAK cocktail may be reduced, and/or (4) the loss of signal due to degradation and/or inactivation of G3PDH may be measured and taken into account. However, most of the small molecules and other agents of interest to high-throughput screening groups would not be significantly affected by exposure to such low levels of a reducing agent. Protease inhibitors would not be likely to have any effect on such compounds.

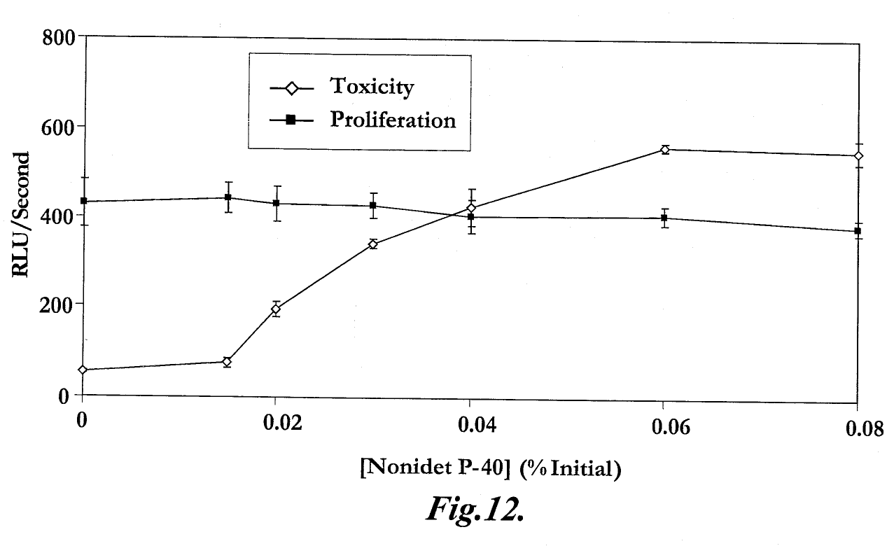

FIG. 12 illustrates the use of cytotoxicity/proliferation mode to measure both the cytotoxic effects and the total cell number after addition of the detergent Nonidet P-40 to the mammalian cell line 841CON. Similar results have been obtained with the PC-3 prostate-cancer and T24 bladder-cancer cell lines. In these experiments, the detergent is used as both the toxin and the final lytic agent. Thus the cytotoxicity signal in FIG. 12 represents the signal obtained after the indicated quantities of detergent were added to the cells, and the proliferation signal in the same figure represents the signals obtained after an additional 0.2% Nonidet P-40 was added to all the cells. Nonidet P-40 is a detergent that has an effect on the DeathTRAK signal—i.e., it reduces the signal by an amount which varies with the concentration up to approximately 45% inhibition at 0.1% Nonidet P-40, but changes very little above approximately 0.1%. Thus Nonidet P-40 can be used as the universal lytic agent for measuring proliferation of mammalian cells, provided that if it is desired to compare the cytotoxicity and proliferation signals, the final signal must be corrected for the inhibitory effect of the detergent. If the final detergent concentration used is above approximately 0.1%, then this correction will consist essentially of multiplication by a constant. However, if proliferation signals alone are to be compared, this correction is not necessary. Note that in FIG. 12, the proliferation signal is fairly constant. This is because each experiment began with the same number of cells seeded into each well. Thus the signal due to addition of the initial aliquot of detergent, as specified on the X-axis (cytotoxicity signal), added to the signal caused by the lytic aliquot of detergent (0.2%), yields a constant which is proportional to the cell number in the well at the beginning of the experiment.

Methods and compositions for the experiment illustrated in FIG. 12, and similar experiments with PC-3 and T24 cells, were as follows:

Cells were grown as for EXAMPLE 1 and plated at a density of 1000 cells in 50 μL into individual wells of a 96-well white luminance microtiter plate, and then grown overnight. The volume in the morning was measured as approximately 40 μL per well. Since DeathTRAK is fully compatible with cell-culture media and growing cells, no washes were performed before initiation of the DeathTRAK assay. The toxins (which were simply the detergent Nonidet P-40 in this case, but could be drug candidate molecules or members of a chemical library) were added in 4.4 μL. (This detergent acts very quickly, and no further incubation was necessary; however, if an incubation were desired in order to give potential toxins time to act, then the user has the option of using the protective cocktail described above containing dithiothreitol and PICguws. This cocktail could be either added separately when the toxins are added, combined with the toxins in solution, or added to the original cell suspension; in the latter case, overnight growth would not be recommended, since the dithiothreitol would probably be oxidized during the overnight incubation.) Following addition of the toxin/detergent, 40 μL of the DeathTRAK cocktail was added. The cocktail composition was as follows:

TABLE III
Preferred Composition of DeathTRAK Cocktail for
Cytotoxicity Measurement

| | Volume |
|---|---|
| Material | (for 40-μL final volume) |
| Non-Labile Cocktail (explained below) | 37.75 μL |
| Reconstituted ATP Assay | 1.68 μL |
| H₂O | 0.44 μL |
| Phosphoglycerokinase stock (undiluted) | 0.008 μL |
| 100 mM ADP | 0.113 μL |

Since some of these quantities are difficult to measure, and for reproducibility purposes, the cocktail is generally made up for multiple wells in a single vessel; for example, in the current experiment, enough cocktail was made for 45 wells, as follows:
1.704 mL Non-Labile Cocktail
76 μL reconstituted ATP assay
20 μL H₂O
0.36 μL phosphoglycerokinase
5.1 μL 100 mM ADP

Following addition of the DeathTRAK cocktail, luminance of the samples was read for 5 minutes (841CON) or 2.5 minutes (PC-3). In general this step may be carried out for 0.1-10 minutes, depending primarily on the cycle speed of the luminometer being used. The lytic agent was then added: 0.9 μL of 10% Nonidet P-40 in H₂O. If automated injection is being used, this volume may be scaled up to an appropriate volume for automated injection (5 μL or more), using a correspondingly lower concentration of the detergent, without appreciable effect on the assay. Following addition of the lytic agent, the luminance was read again for the proliferation readout. The data were reduced by linear regression.

Composition of the Non-Labile Cocktail (NLC) was as follows:

TABLE IV
Composition of Non-Labile DeathTRAK Cocktail for Cytotoxicity,
Proliferation/Viability, or Combined Mode Measurements

| Material | Volume |
|---|---|
| IMDM growth medium | 4.68 mL |
| PBS | 2.535 mL |
| 4XGP mixture, described above under | 1.2675 mL |
| EXAMPLE 1 | |
| ATP Assay Diluent | 5.703 mL |
| 1M dithiothreitol | 0.006 mL |
| 100 mM ADP | 0.0045 mL |

Note that the ADP is an optional ingredient in the NLC. In some experiments, as in this Example, additional ADP is provided in the final cocktail. In general, the ADP may be provided in the NLC for convenience, or added as the final cocktail is made up so as to control the final concentration precisely and protect the ADP from any degradation caused by components of the NLC. The NLC may be stored for several months at −80 C with little change in activity if the ADP is stored separately and added on the day of use, or approximately 1-3 weeks if the ADP is included.

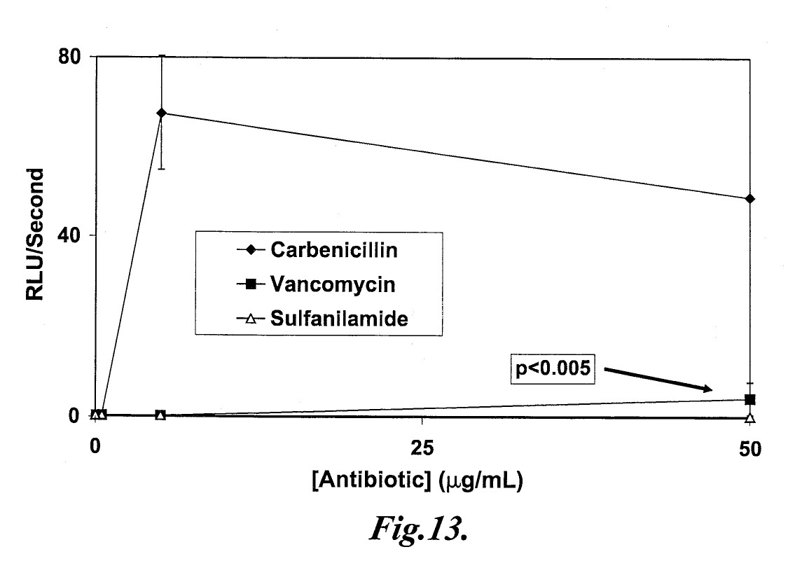

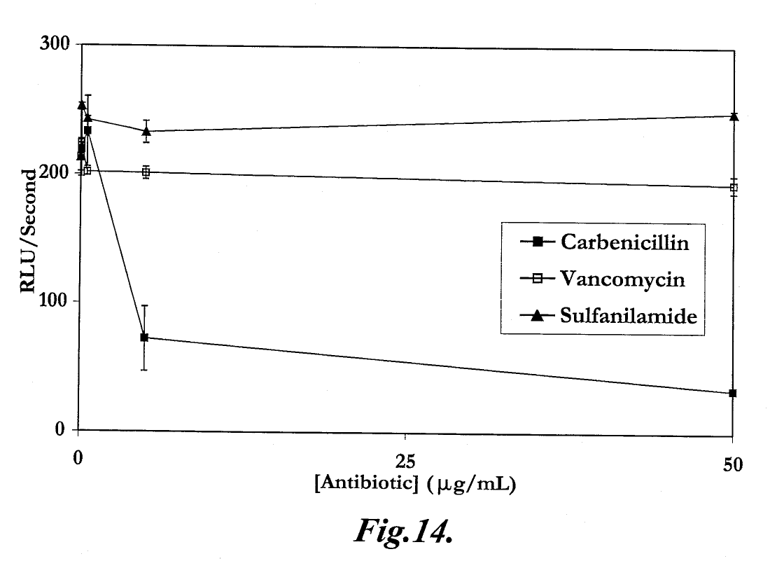

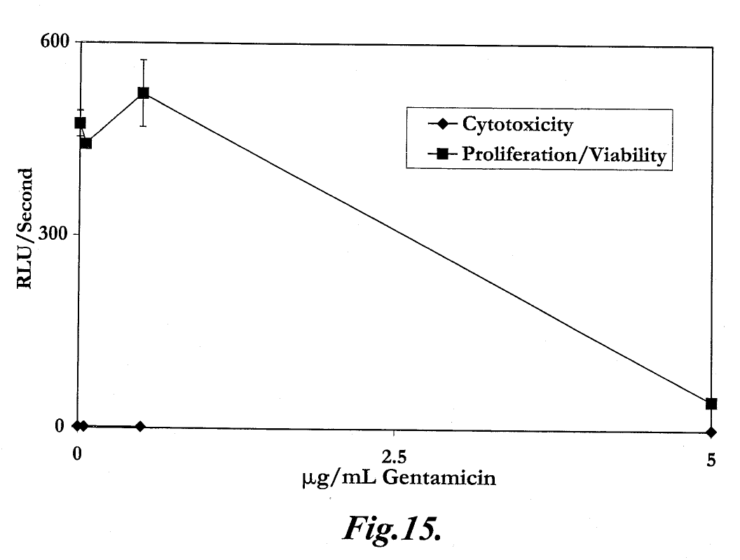

FIGS. 13-15 illustrate the use of cytotoxicity/proliferation mode to characterize the effects of various antibiotics on E. coli cells. First, the cytotoxicity signal was obtained by adding the DeathTRAK reagent cocktail directly to the toxicity reaction, three hours after the antibiotics were added to the E. coli culture. The luminance was measured and recorded (FIG. 13), whereupon the lytic agent was added, and the luminance was immediately measured again (FIG. 14). As is apparent from the figures, carbenicillin exhibited both strong cytotoxicity and a strong inhibitory effect on proliferation/viability. Vancomycin exhibited slight but statistically significant toxicity, and slight inhibition of proliferation/viability which is not significant by t-test but passes a rank-sum test. Sulfanilamide exhibited no toxicity or effect on proliferation/viability. Colony-forming unit (CFU) tests, in which the cultures were plated at various dilutions, confirmed that both carbenicillin and vancomycin were toxic, while sulfanilamide exhibited no toxicity by CFU assay at these concentrations. FIG. 15 shows the results of a similar experiment with gentamicin, which exhibited a strong effect on proliferation/viability, but no cytotoxicity in 90 minutes. In each case, the results of DeathTRAK are predictive of the known mechanisms of the respective antibiotics, as illustrated by the following table, where “+” indicates a cytotoxic or antiproliferative effect:

TABLE V
Antibiotic Effects on E. coli Measured and Mechanistic
Information Obtained Using DeathTRAK

| | | Proliferation/ | |
|---|---|---|---|
| Antibiotic | Cytotoxicity | Viability | Mechanism |
| Carbenicillin | + | + | Inhibits cell-wall synthesis |
| Vancomycin | + | + | Interferes with cell-wall |
| | | | cross-linking |
| Gentamicin | − | + | Inhibits protein synthesis, |
| | | | intracellular |
| Sulfanilamide | − | − | Not toxic at these |
| | | | concentrations |

Thus a clear advantage of the DeathTRAK method is that not only may effective antibacterial compounds be identified, but mechanistic information about the candidate antibiotics may also be collected at the same time, in an assay rapid enough for use in high-throughput screening.

Compositions for Bacterial DeathTRAK

Total Lysis of Gram Negatives

The Gram negative bacterium E. coli was killed in the total-lysis step with a mixture of polymyxin B and chicken lysozyme. Both components were necessary for the lysis to occur, and titration experiments established the optimal concentration of polymyxin B as ˜300 units/mL and the optimal concentration of lysozyme as ˜2.5% final. It will be evident to one skilled in the art that other pore-forming agents and other enzymes may be successfully substituted for polymyxin B and lysozyme, respectively, in this system.

Total-Lysis Experiments

E. coli were grown overnight in LB from refrigerated cultures, washed in LB, and resuspended to a final A₆₀₀ of 2.18. Lytic agents (polymyxin B, 30200 units/mL in PBS, lysozyme, 5% in PBS) were added (5 μL each to 45 μL of culture in luminance microtiter plate), whereupon 45 μL of DeathTRAK cocktail made up as for cytotoxicity/proliferation experiments was added and the luminance was read.

Preparation of E. coli

E. coli (strain K1, obtained from Dr. Craig Rubens of Children's Hospital and Regional Medical Center, Seattle, Wash.) were inoculated from a frozen permanent into 1-2 mL of LB, grown overnight, diluted 1:20 into LB, grown a further 106 minutes at 37 C with 240 rpm shaking, harvested by centrifugation, washed twice with LB, and resuspended to a final A₆₀₀ Of 1.549. It was determined by colony-forming unit assays that an A₆₀₀ of 2.18 corresponds to ˜3.02×10⁸ cells/mL. This was diluted to 200,000/mL and a 10% volume of 10 mM dithiothreitol and 1% protease inhibitor cocktail in LB was added (yielding 0.1% protease inhibitor cocktail after mixing). 55 μL of this mixture was distributed to each test well of a 96-well white luminance microtiter plate, whereupon 5 μL of antibiotic or PBS (vehicle) was added. The plate was shaken at 240 rpm for 3 hours at 37 C. 40 μL of DeathTRAK cocktail was then added and luminance was read for ˜8 minutes. The lytic agent was then made up as equal parts 6000 units/mL polymyxin B and 50% lysozyme, both in PBS. 10 μL of this lytic agent was added to each well and the second luminance readout (proliferation/viability) was taken.

Results of Measurement of Cytotoxicity/Proliferation of Gram Positive Bacteria with DeathTRAK

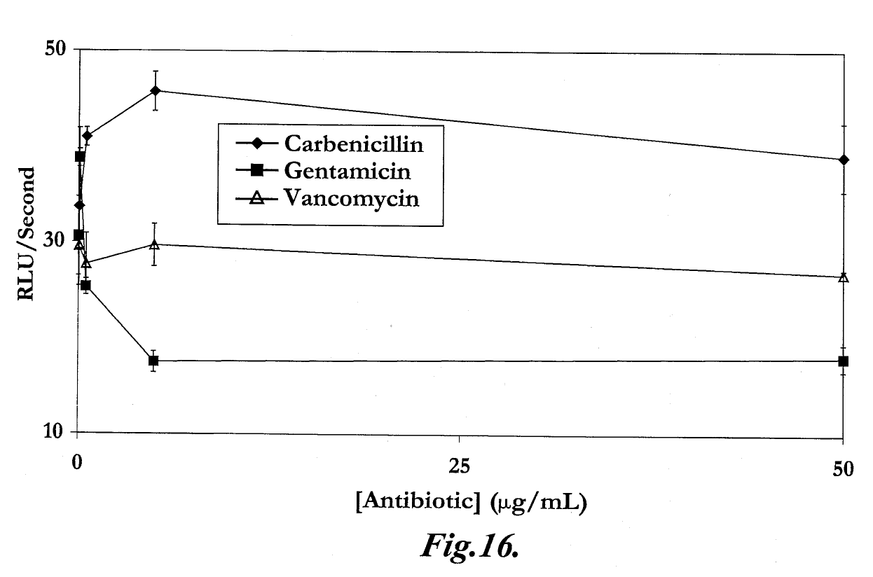

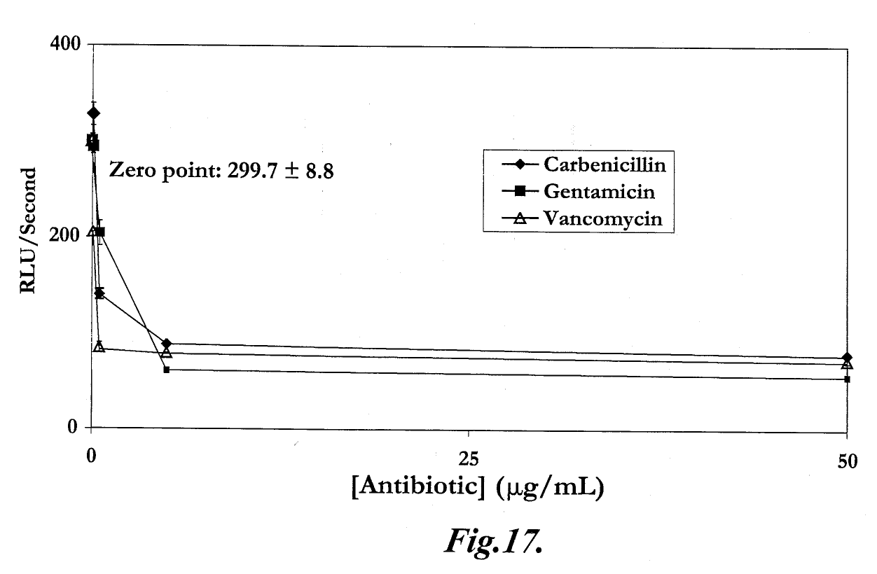

FIGS. 16 and 17 illustrate the use of cytotoxicity/proliferation mode to characterize the effects of various antibiotics on Group-A streptococci. These are Gram positive organisms. Since they lack an outer membrane and have much thicker cell walls, the effects of the antibiotics are, as expected, different from those observed with E. coli. However, both cytotoxicity and proliferation effects are seen. Thus carbenicillin is identified as a lytic agent, as it is with Gram negatives. Vancomycin exhibited little or no direct cytotoxicity, but yielded a very strong reduction in the proliferation/viability signal. This interesting result may indicate either that vancomycin has other effects on Gram positives, in addition to effects on cell wall cross-linking, or that with the very thick cell walls of Gram positives, the degree of inhibition required to kill the cell is lower than that needed to cause overt lysis. Finally, the result with gentamicin is distinctive. This compound yielded a negative toxicity signal, relative to the no-antibiotic signal. The explanation is that these Gram positives slowly leak G3PDH. Thus the cytotoxicity signals seen with the Group A streptococci represent the sum of G3PDH released by lysed cells and G3PDH leaking from live cells. In the case of gentamicin, which causes little lysis but strongly inhibits growth, there is no appreciable cytotoxicity, and there are also fewer cells present to leak enzyme; thus the apparent cytotoxicity signal is lower than that seen without antibiotic. However, these effects are clarified by the proliferation/viability data, which clearly show that gentamicin is strongly toxic. Thus in the dual mode, it is very unlikely that any useful non-lytic toxin would be missed due to the leakiness (because it would be identified in the viability wing), while the only compound known to cause rapid lysis of Group A streptococci (carbenicillin) was correctly identified in the cytotoxicity wing of this experiment. It should be noted that E. coli do not exhibit this leakiness (see FIG. 10).

Methods and Compositions for DeathTRAK Cytotoxicity/Proliferation Mode (Gram Positive Bacteria)

Group-A Streptococci obtained from Dr. Craig Rubens of Children's Hospital and Regional Medical Center, Seattle, Wash. were inoculated from a frozen permanent and grown overnight in THY medium, then diluted 1:10 into THY and grown an additional 136 minutes, harvested, and washed twice into 50% of the growth volume. The A₆₀₀ was 0.356. They were diluted to 4,000,000/mL on the basis of the A₆₀₀/cell count relationship determined above for Gram negatives; however, these Gram positives are somewhat larger and therefore yield correspondingly fewer cells per A₆₀₀ unit. After washing the cells were grown for 90 minutes at 37 C with 240 rpm shaking. Antibiotics or PBS (vehicle) were then added in 5 μL, and the protective cocktail containing dithiothreitol and protease inhibitor cocktail was added in a further 5 μL. The cells were incubated at 37 C with 240 rpm shaking for a further 90 minutes, whereupon the DeathTRAK cocktail was added and the cytotoxicity read as described above for Gram negatives. The total-lysis agent, 10 μL of 2% Nonidet P-40, was then added, and the proliferation/viability signal was read as above for Gram negatives.

### Example 9

### High-Throughput Screening for Cytotoxicity and/or Membrane Damage and/or Proliferation

The DeathTRAK assay or another coupled luminescent assay as described below under EXAMPLE 14 in high-throughput screening is a completely homogeneous assay, in that (1) only a single injection of the cocktail would be necessary, and (2) no manipulation by humans would be required after the assay cocktail was loaded for injection and the plate was placed in the luminometer. DeathTRAK can also be used with scintillation counters, spectrophotometers, and fluorometers. Again, the cocktail may be injected before, during, or after introduction of the potentially cytotoxic reagent. In the most rapid possible mode, the potentially cytotoxic reagent may be added simultaneously with the cocktail and the luminance readout could be taken within minutes, or possibly even in less than one minute. The proliferation mode as described in EXAMPLE 7 may also be useful in an HTS environment, especially since the sensitivity is greater than that of ATP-release assays. As described above, the readout of DeathTRAK and related assays may be analyzed by performing linear fits to determine the rate of increase of the luminance signal with time, or, more conveniently for high-throughput assays, by taking a single read at a constant, predetermined time after injection of the reagent cocktail and using this as the luminance readout.

### Example 10

### Screening for Drug Resistance/Sensitivity

Samples of a patient's cells, or a culture of an infectious agent from a patient, may be screened against various drugs using DeathTRAK or another coupled luminescent assay as described below under EXAMPLE 14 to determine sensitivity and/or resistance to the drugs. This may be accomplished in cytotoxicity, proliferation, or combined mode.

### Example 11

### Measurement of Apoptosis

Determination of apoptosis may be carried out just as other forms of cytotoxicity are measured, using DeathTRAK or another coupled luminescent assay as described below under EXAMPLE 14. Alternatively it may be possible to take advantage of the fact that apoptosis is associated with increased levels of G3PDH in the nucleus (Carlile et al., Mol. Pharmacol. 57:2-12). This may be done, for example, by lysing equal numbers of cells and comparing levels of G3PDH activity via DeathTRAK. In another embodiment, apoptosis could be distinguished from necrosis by use of a simultaneous assay which is specific for apoptosis, such as the TUNEL assay. Since apoptosis is largely an internal cellular process, membrane rupture is a relatively late event in apoptosis compared with DNA fragmentation, caspase activation, and other associated events. TUNEL and caspase assays would therefore be expected to respond to apoptotic events on a different timescale from release assays, while necrosis would likely lead to a signal in a coupled-luminescent release assay, but not in an apoptotic assay. Comparison of the time-dependence of the signals from the two assays should therefore allow the user to separate apoptotic from necrotic cell death.

### Example 12

### Monitoring for

### Sterility or Bioburden

Use of DeathTRAK or another coupled luminescent assay as described below under EXAMPLE 14 for sterility or bioburden monitoring would be similar to use in proliferation mode. Liquid samples or swab samples may be tested by addition of a lytic reagent followed by the coupled luminescent assay. This method is much more sensitive than other liquid-phase methods in current use.

### Example 13

### Monitoring of Environmental Toxins

In this mode, DeathTRAK or another coupled luminescent assay as described below under EXAMPLE 14 typically would be used in combination with one or more standard cell lines or cell types Testing for shellfish toxins, for example, might involve the use of neuroblastoma cells in combination with reagents to distinguish sodium-channel blockers from enhancers, as previously described (Manger et al., J AOAC Int. 78:521-7, 1995). The MTT assay used in that work would be replaced by DeathTRAK or another coupled luminescent assay as described below under EXAMPLE 14.

### Example 14

### Extension to Other Coupled Luminescent Enzyme Systems

Coupled luminescent enzyme assays, as described herein, are extensible to other combinations of enzymes (EXAMPLE 5: Aldolase-DeathTRAK). Other systems in which coupled luminescent assays of cytotoxicity are possible include:

(1) “Reverse” DeathTRAK in which G3PDH is supplied in the cocktail, but PGK is omitted and is the reagent under test.

(2) Measurement of release of pyruvate kinase, Like G3PDH, this enzyme makes ATP by phosphorylating ADP. The substrate of pyruvate kinase, phosphoenol pyruvate, is quite unstable but could be supplied, or generated in situ by the action of enolase on 2-phosphoglycerate.

(3) Measurement of release of lactate dehydrogenase. This enzyme interconverts pyruvate and lactate, simultaneously interconverting the reduced and oxidized forms of nicotinamide adenine dinucleotide. The reduced form, abbreviated NADH, is a substrate for certain bacterial luciferases. Some of these luciferases react very rapidly with NADH and may work better in a glow luminescence reaction than in a flash reaction. Both kinds of reactions are compatible with the coupled luminescent assay methods discussed herein. Either pyruvate or lactate may be supplied in the cocktail, and either disappearance or appearance of NADH would be measured by coupling with bacterial luciferase, respectively. This system may also be coupled to enzymes that generate pyruvate or lactate, or enzymes of the tricarboxylic acid cycle involved in NADH metabolism.

(4) Measurement of G3PDH release by measurement of NADH in a similar manner. Again, this system may be coupled to enzymes that generate G3P or 1,3 diphosphoglycerate (the product of G3PDH oxidative phosphorylation of G3P).

(5) Measurement of release of any kinase. Such kinases consume ATP, which would result in a detectable decrease in a luminance signal. However these enzymes can also be run “backwards” if the appropriate phosphorylated substrate is supplied, thus generating ATP.

(6) Measurement of release of isocitrate dehydrogenase by observing appearance/disappearance of NADH by flash luminescence.

(7) Measurement of release of succinyl-CoA synthase by coupling of ATP (or GTP) appearance or disappearance with luciferase luminescence.

(8) Measurement of phosphatases released by lysed cells, or phosphatases retained in the membranes of lysed cells, by the method described in EXAMPLE 16. Note that the PhosTRAK components, like DeathTRAK, are compatible with the presence of live cells. In an alternative embodiment, the free phosphate released by lysed cells could be measured as in EXAMPLES 16 and 17 to quantify those cells.

(9) In general, measurement of release of any enzyme which produces and/or destroys ATP, NADH, or another molecule may be used as a luminescent substrate by luciferases.

### Example 15

### Extension of Coupled Luminescent Assays to Uses Other than Measurement of Cytotoxicity, Membrane Damage, and Proliferation

Cytotoxicity is not the only possible target of coupled luminescent assays. For example, specific kinases are of great interest in cancer research. Current specific kinase assays are mostly laborious, involving radioactively labeled substrates and physical separation of the phosphorylated target from the label, followed by scintillation counting. Instead, the DeathTRAK invention may be used to assay for specific kinase activity in at least one of two ways:

(1) ATP, ATP assay cocktail, and the target of the specific kinase are supplied in a master mix. If the specific kinase is present, the luminance signal will decrease with time as ATP is exhausted.

(2) If a positive signal is desired, the assay can be run in the reverse direction. This requires prior synthesis of the phosphorylated target, which will be problematic in some cases. The phosphorylated target, ADP, and ATP assay cocktail would be supplied. If the specific kinase is present, ATP will be created by the reverse action of the kinase and the luminance will increase with time.

Other uses of the DeathTRAK invention are:

(3) A coupled luminescent assay can be used for ultrasensitive detection of specific free amino acids. The corresponding aminoacyl-tRNA synthetases would be provided, possibly with a mixture of tRNAs (some of these enzymes do not require tRNA for the charging step). ATP would be consumed by charging if the specific amino acid were present, causing a decrease in the luminance signal.

(4) Clinical laboratories often require ultrasensitive assays for enzymes such as lactate dehydrogenase and isocitrate dehydrogenase. These can be coupled to production or consumption of ATP by methods described above to give a luminescent readout. This method would probably be more sensitive than EIA methods.

(5) A number of types of ATPases have been characterized, including sodium/potassium-dependent, F₀/F₁, and proton-pump ATPases, all of which are of great biological importance in many organisms. In some cases, coupling of these ATP-destroying and/or creating activities to luminescent detection of ATP could represent an improved method of assaying these ATPases.

(6) One of the major battles in the struggle to defeat the trypanosome is the effort to find specific inhibitors of the glycolytic enzymes of these organisms, which differ significantly from the corresponding mammalian enzymes. Since DeathTRAK is really a G3PDH assay (and can be used as a PGK assay), it could be useful in high-throughput screening for differential inhibition of these enzymes (e.g., Bressi J C, Choe J, Hough M T, Buckner F S, Van Voorhis W C, Verlinde C L, Hol W G, Gelb M B, J Med Chem 2000 Nov. 2; 43(22), 4135-50).

### Example 16

### Measurement of Phosphatase Activity by PhosTRAK

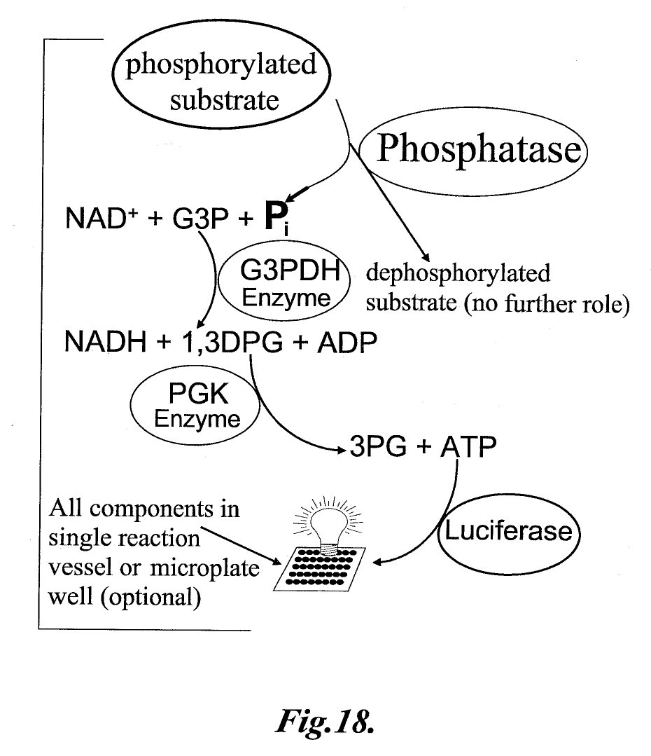

PhosTRAK is an assay for free phosphate, and therefore for the activity of phosphatases, enzymes which liberate free phosphate. The reaction scheme of PhosTRAK is nearly identical with that of DeathTRAK as can be seen from FIG. 18, which is similar to FIG. 1, the schematic representation of DeathTRAK. The critical difference is that in PhosTRAK, the reagent being measured is free phosphate, which must therefore be the limiting reagent. This implies that G3PDH, which is the limiting reagent in DeathTRAK, must be present in PhosTRAK, and it is therefore supplied in the cocktail. Conversely, the reagents used for PhosTRAK (other than the test sample) should be made as free of phosphate as is practicable, in order to reduce the background signal from endogenous phosphate in the cocktail; however, even if substantial phosphate contamination is present, it is still possible to perform PhosTRAK by subtracting the constant background signal due to endogenous phosphate or phosphate contaminating the phosphatase preparation (or other substances added by the user) from the time-dependent increase in luminance due to release of phosphate by the phosphatase.

The buffer used for phosphatase assays may be identical or similar to that used for the DeathTRAK cytotoxicity assay, with the following exceptions:

(A) Buffers, enzymes, and other components should be rendered as free of inorganic phosphate ion as is practicable. Instead of the sodium phosphate buffer used for DeathTRAK, the PhosTRAK buffer is a Tris-based buffer, described below as “reduced-phosphate cocktail.”

(B) G3PDH is supplied in the PhosTRAK cocktail.

(C) Buffer components, small molecules, cofactors, and other elements essential to measurement of the activity of the phosphatase under study, made as free of phosphate as is practicable, are added to the reaction mixture.

A phosphatase assay performed in this manner may be used for the purposes of identifying inhibitors, enhancers, or other modulators of phosphatase activity (for example, molecules which change the pH profile or substrate-response profile of a phosphatase, or alter the manner in which the phosphatase responds to another regulatory molecule). Such a phosphatase assay may be used in conjunction with high-throughput screening methods, such as robotics, with or without automated injection and transfers, for example, to screen or test chemical libraries for inhibiting, enhancing, or modulatory activities against phosphatases.

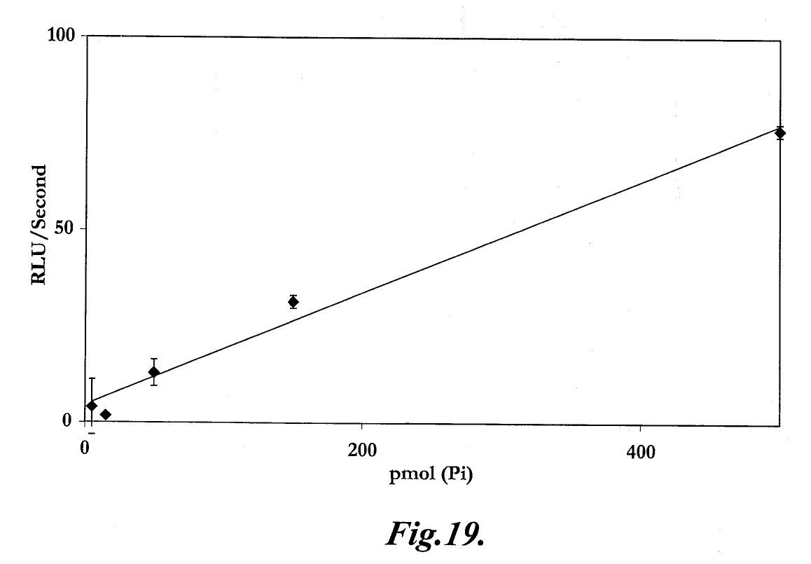

FIG. 19 shows the results of an experiment in which free phosphate was detected using PhosTRAK. A reduced-phosphate cocktail was made up as follows:
75 μL 1 M Tri s-HCL pH 7.4
647 μL H₂O
1.25 μL PGK diluted 1:10,000 with PGK diluent
570 μL ATP Assay diluent
63 μL ATP Assay
0.6 μL 1M dithiothreitol
0.75 μL G3PDH, Sigma catalog #G-9263, diluted 1:100 with G3PDH diluent
121 μL of special 4×GP cocktail made up without free phosphate G3PDH diluent was made up as follows:
1000 parts phosphate-free PGK diluent
1 part 1M dithiothreitol

Composition of Special 4×GP cocktail without free phosphate was as follows:
2 mL 5× phosphate-free PGK diluent
10 μL IM dithiothreitol
100 μL 100 mM NAD+
286 μL H₂O

Composition of 5× phosphate-free PGK diluent
666 μL triethanolamine
2.5 mL 1 M Tris pH 7.4
259 μL 193 mM EDTA pH 8.0
5 mg five-times recrystallized BSA
Titrated to pH 7.3 with HCL, made up to 10 mL with H₂O

The reduced-phosphate cocktail was supplemented with 0.9 μL of 10 mM ADP and was made 3 μM in glyceraldehyde-3-phosphate (Sigma catalog #G5251). 45 μL of the reduced cocktail was distributed into each test well of a luminescent microtiter plate. 5 μL of phosphate solution or H₂O was added to yield the total amounts of free phosphate indicated in the X-axis of FIG. 19, and the luminance was read for six minutes. The reactions were done in triplicate. Data from the first 160 seconds of the run were taken for analysis. The background level due to free phosphate present in the cocktail was subtracted from the data. Measurements of the signal-to-background ratio at various concentrations of glyceraldehyde-3-phosphate showed that this background is largely due to free phosphate present in the glyceraldehyde-3-phosphate preparation. The use of purified glyceraldehyde-3-phosphate would therefore ameliorate this background problem. (As an alternative, making glyceraldehyde-3-phosphate from glyceraldehyde-3-phosphate diethyl acetal barium salt, a new product from Sigma (catalog #G-5376), should produce glyceraldehyde-3-phosphate with a lower concentration of contaminating free phosphate.) In these experiments the signal-to-background ratio increased with decreasing glyceraldehyde-3-phosphate down to 3 μM. The size of the background signal indicated that 300-500 nM free phosphate was added with 3 μM glyceraldehyde-3-phosphate, for a contamination level of 10-15%. It should be possible to reduce this by at least five- to ten-fold by purifying the glyceraldehyde-3-phosphate prior to use in the assay.

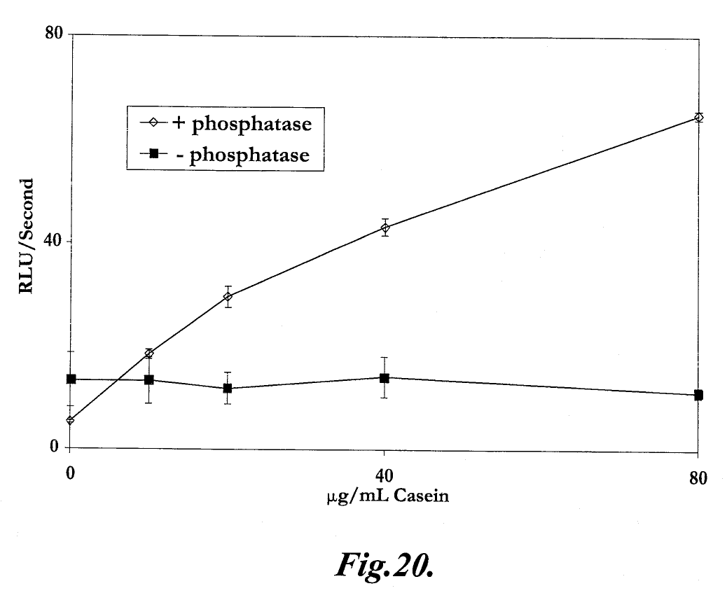

FIG. 20 shows the results of an experiment in which the activity of λ-phosphatase was detected with PhosTRAK. For this experiment the following cocktail was made:
75 μL 1M Tris-HCL pH 7.4
1.25 μL PGK diluted 1:10,000 with PGK diluent
121 μL special 4×GP phosphate-free cocktail
570 μL ATP Assay diluent
63 μL ATP Assay
3 μL 1M dithiothreitol
0.75 μL G3PDH diluted 1:100 with G3PDH diluent
0.9 μL 10 mM ADP
1.5 μL 2.88 mM glyceraldehyde-3-phosphate
6 μL 500 mM MnCl2
507.6 μL H₂O

To 600 μL of this cocktail was added 1 μL of λ-phosphatase (Sigma catalog #P-9614). Another 600-μL aliquot did not receive phosphatase. 45 μL of each of these respective aliquots was transferred in duplicate to wells of a luminance microtiter plate, and the indicated final concentrations of purified α-casein were achieved by addition of 5 μL of appropriate dilutions of casein in H₂O.

The activity of the phosphatase calcineurin was also detected with PhosTRAK. A phosphatase non-labile cocktail was made up as follows:
225 μL 1M Tris-HCl pH 7.4
363 μL special 4×GP, as used for assaying λ-phosphatase
1.71 mL ATP Assay diluent
9 μL 1 M dithiothreitol
2.7 μL 100 mM ADP
4.5 μL 2.8 mM glyceraldehyde-3-phosphate
1,543 mL H₂O

The final reaction cocktail was made up as follows:
1.285 mL above-described non-labile cocktail
63 μL ATP Assay
1.25 μL PGK diluted 1:10,000 with PGK diluent
0.75 μL G3PDH diluted 1:100 with G3PDH diluent

Calmodulin was made up to 2.50 nM in a reaction buffer supplied with a kit from Calbiochem (catalog #207005). 50 μL of this mixture was distributed to wells of a luminance microtiter plate. A 1% volume of calcineurin (8 units/μL) from the same kit was then added to the calmodulin solution and 50 μL of this mixture were transferred to positive wells. Negative wells did not contain calcineurin. Purified a-casein in PCK diluent was added to 40 μg/mL (final concentration) in 5 μL, whereupon the reactions were incubated for 30 minutes at room temperature. After the incubation, the final reaction cocktail was added (45 μL) to each well and the luminance was read.

The results of the assay were: +calcineurin, 2.99±0.47 RLU/Sec; −calcineurin, 2.30±0.06 RLU/Sec. The results of the assay show that a signal due to calcineurin is detected, but the background signal due to endogenous free phosphate is high. The use of purified glyceraldehyde-3-phosphate would improve the signal-to-background ratio in the detection of calcineurin activity.

### Example 17

### Other Applications of Phosphate and Phosphatase Detection by PhosTRAK

Detection of free phosphate by the methods described under EXAMPLE 16 would be useful in a number of areas. Apart from measurement of phosphatase activity, detection of free phosphate could have applications in measurement or detection of other enzymatic, chemical, or biochemical reactions, including (A) pyrophosphatase activity, (B) spontaneous or catalyzed breakdown of nucleotide triphosphates, phosphorylated proteins, or other phosphate esters or diesters. PhosTRAK can be coupled to other systems in which free phosphate is a substrate, intermediate, or product to help in monitoring such reactions, or could be coupled to the action of a pyrophosphatase to allow quantification of any activity involving release of pyrophosphate. However, care must be taken in quantification of pyrophosphate, since the PhosTRAK system itself involves production of pyrophosphate, which could represent a background signal for which a correction would be made. Detection of free phosphate could also be useful in environmental monitoring. Eutrophication of bodies of water is often accompanied by a rise in the phosphate concentration. The presence of phosphate is frequently representative of the release into ground water of certain detergents and/or other compounds.

In a further embodiment of the present invention, the coupled luminescent detection system of PhosTRAK could be used to detect the activity of a phosphatase coupled or conjugated to other proteins or other molecules in ELISA, PCR, RT-PCR, Westerns, immunohistochemistry, in situ hybridization, or other techniques involving detection of a target or event by enzymatic labeling.

In a further embodiment of the present invention, the coupled luminescent detection of PhosTRAK could be used to detect the presence of inhibitors of phosphatases in the environment, in food samples, in research situations, or in other cases in which phosphatase inhibitors are of importance. Such an approach might be used, for example, to detect the toxin okadaic acid in shellfish extracts.

### Example 18

### Detection of NAD⁺ by Coupled Luminescence

NAD⁺ may be detected by a reaction scheme which is substantially similar to the DeathTRAK and PhosTRAK schemes depicted in FIGS. 1 and 18, respectively. For detection of NAD⁺, the NAD⁺ that is supplied in the DeathTRAK and PhosTRAK schemes is instead omitted, and both G3PDH and inorganic phosphate are supplied reagents. Thus light production via the same pathway depends critically on the generation or presence of NAD⁺ from another source, which can be direct addition of NAD⁺, a medical, environmental, or other sample containing NAD⁺, or generation of NAD⁺ by the action of an enzyme or enzymes in oxidizing NADH, usually with simultaneous reduction of a substrate molecule.

An NAD⁺ assay may be assembled and used as follows:
4×LGP Cocktail (for approximately 0.63 mL):
   0.5 mL 5×PGK diluent;
   0.00625 mL 1 M dithiothreitol;
   0.025 mL 1 mM NADH;
   0.026 mL glyceraldehyde-3-phosphate (50 mg/mL);
   0.0725 mL dH₂O.
G-diluent (for approximately 10 mL):
   2 mL 5×PGK diluent;
   8 mL dH₂O;
   0.01 mL 1M dithiothreitol.
LT Cocktail (for approximately 2 mL):
   0.624 mL Tris-buffered saline;
   0.338 mL phosphate-buffered saline;
   0.169 mL 4×LGP cocktail (above);
   0.76 mL ATP Assay Diluent (Sigma FLAAB);
   0.0024 mL 1M dithiothreitol;
   0.005 mL 100 mM ADP;
   0.084 mL ATP Assay Mix (Sigma FLAAM) (add last).
Enzyme Cocktail:
   0.08 mL phosphoglycerokinase, diluted 1:100 with G-diluent;
   0.001 mL glyceraldehyde-3-phosphate dehydrogenase, diluted 1:1000 with G-diluent.
Final Reaction Cocktail:
   0.4 mL LT cocktail (above)
   0.064 mL enzyme cocktail (above)

Assay Procedure: 0.001 mL of 10 μM NAD⁺ was distributed to three wells of a white luminance microtiter plate, while three control wells received no addition. Subsequently 0.05 mL of the Final Reaction Cocktail was added to all six wells, the plate was gently tapped 15-20 times within a few seconds to mix the components, and the plate was transferred to an LKB96P luminometer. The results are shown in Table VI. The 1 nanomole of NAD⁺ present in the positive wells is very easily distinguished from the background signal. Moreover, the background signal is static, or slightly negative with time, whereas the NAD⁺ signal is strongly increasing with time. It is clear that 1 nanomole is far higher than the actual detection limit for NAD⁺ in this system. The coefficients of variation are very small, in the range of 2-5%.

TABLE VI
NAD⁺ Detection by Coupled Luminescent Assay

| Observed Luminance: | Initiation | 177 Seconds | 298 Seconds |
|---|---|---|---|
| +1 nmol NAD⁺ | 47272 ± 1432 | 90955 ± 3181 | 105348 ± 4208 |
| No NAD⁺ | 19012 ± 482 | 18824 ± 486 | 18892 ± 345 |

### Example 19

### Detection and/or Measurement of Lactate Dehydrogenase Enzymatic Activity by Coupled Luminescence

Lactate dehydrogenase (LDH) catalyzes the simultaneous reduction of pyruvate (or pyruvic acid) and oxidation of NADH. Although this is the reverse of the canonical reaction sequence for which the enzyme is named, it proceeds efficiently. This reaction generates NAD⁺, which may be detected by the scheme given in EXAMPLE 18. This example employs an enhanced enzyme cocktail, which may also be used in other procedures requiring detection and/or quantification of NAD⁺. The reaction scheme requires pyruvate (or pyruvic acid) as a substrate for LDH. This example is an embodiment of the reaction series presented in FIG. 21.

LT Cocktail was made as in EXAMPLE 18. The enhanced enzyme cocktail was as follows:

Enhanced Enzyme Cocktail:
0.08 mL 1:100 phosphoglycerokinase (diluted in G-diluent);
0.005 mL 1:1000 glyceraldehyde-3-phosphate dehydrogenase (diluted in G-diluent).

LDH Buffer:
1% Bovine Serum Albumin (purified Fraction V) dissolved in Tris-Buffered Saline, pH. 7.4.

The final reaction cocktail was as follows:
0.5 mL LT cocktail plus 0.085 mL enhanced enzyme cocktail.

Procedure: Four wells of a luminance microtiter plate each received 0.001 mL 300 mM sodium pyruvate (LDH substrate), while four control wells received no addition. Two of the control wells and two of the substrate wells received 0.005 mL LDH Buffer, while the other two control wells and the other two substrate wells each received 0.005 mL LDH, previously diluted in LDH Buffer (Tris-buffered saline, pH 7.4, with 1% Fraction V BSA) to 12.6 units/mL. The plate was gently tapped 15-20 times for mixing and placed in the LKB96P luminometer for reading. The results are shown in Table VII. Wells without pyruvate, and wells containing pyruvate but no LDH, yield very small signals, while wells containing both LDH and pyruvate exhibit large, time-dependent luminance changes. The controls without pyruvate indicate that the signal observed is very likely due to LDH activity, rather than a contaminating enzyme, since LDH is one of the few enzymes that oxidizes NADH to NAD⁺ in the presence of pyruvate or pyruvic acid.

TABLE VII
Measurement of Lactate Dehydrogenase Activity by
Coupled Luminescence

| | Initiation | 57 Sec | 126 Sec | 199 Sec |
|---|---|---|---|---|
| +LDH, +Pyruvate | 37241 | 69768 | 103568 | 136677 |
| +LDH, −Pyruvate | 20689 | 20738 | 21088 | 21775 |
| −LDH, +Pyruvate | 16824 | 15683 | 14585 | 14060 |
| −LDH, −Pyruvate | 16986 | 15954 | 14807 | 14154 |

### Example 20

### Use of the LDH/NAD⁺ Reaction to Detect and/or Quantify Cell Death and/or Membrane Damage, and/or Count Live Cells, and/or Separately Quantify Cell Death and/or Membrane Damage and Count Live Cells in the Same Reagent Mixture

Cells with damaged membranes generally release enzymes, including LDH, into the surrounding liquid. Thus the number of dead cells and/or the degree of membrane damage may be assessed by measuring the activity of LDH in the surrounding liquid, using either the methods of EXAMPLE 20 or other coupled luminescent methods. Moreover, coupled luminescent reaction series may be used to count live cells as well, by intentionally causing lysis of the live cells and performing the assay. The lytic agent and reaction cocktail may optionally be supplied in the same reagent aliquot, or added sequentially in either order. Moreover, in the “dual mode”, the assay is performed with an unmodified sample to determine cell death and/or membrane damage, a lytic agent is added, the assay is performed again to determine a total cell count, and the live count is determined by subtracting the “dead” count from the total count. This procedure may optionally be modified to include a mathematical adjustment of the total cell count quantification for the volume change due to addition of the lytic agent and/or any changes the lytic agent may exert on the reaction velocity, which may be predetermined in separate experiments. Standards may optionally be run separately or in parallel to aid in determining absolute cell numbers, the standards consisting of samples of cells in known quantities and/or dilution series of the test enzyme. Any or all of the methods under this example may be used for high-throughput screening of drug candidate molecules in a primary, secondary, or other screening mode; testing patient cells for sensitivity to chemotherapeutic or other drugs; studies of the effects of drugs, drug candidates, molecules with partially or wholly unknown properties, antibodies, proteins, enzymes, enzyme inhibitors, channel proteins, channel blockers, pore formers, detergents, other lytic agents, complement, sera, cytotoxic T lymphocytes, NK cells, NKT cells, macrophages, antibiotics, Tₕ cells, lytic and other viruses, or combinations of these agents; or other purposes related to cell death, membrane damage, and/or live cell number. The method is applicable to all eukaryotic, prokaryotic, and archaebacterial cells or combinations of these.

### Example 21

### Detection and/or Measurement of the Activity of Acetylcholinesterase by Coupled Luminescence Involving Phosphorylation of Acetate by Acetate Kinase or Phosphorylation of Choline by Choline Kinase

Acetylcholinesterase (ACHE) is a critical enzyme involved in neurotransmission, and it is inhibited by nerve gases, certain Alzheimer's drugs, and various pesticides of agricultural importance. Thus methods for detecting and measuring the activity of ACHE have potentially broad applications. It should be noted here that different forms of ACHE may be most useful for different applications. For example, if an insecticide that has been developed as a specific inhibitor of an insect ACHE is the detection target, then the most appropriate choice of ACHE enzyme for that application is likely to be an ACHE purified (or cloned and expressed) from the same or a related species, or possibly from another species entirely with homologous or functionally similar ACHE. In the work reported here the electric-eel enzyme was used. This enzyme is inexpensive and easy to use, and its specificity is very similar to that of the human enzyme, as shown by the tacrine inhibition in this example (tacrine is an Alzheimer's drug).

In the assay mode described here, the activity of ACHE leads via the activity of acetate kinase to a reduction in the ATP concentration, and therefore a decrease in light output. If ACHE is inhibited, more light is detected. This example is an embodiment of the reaction series presented in FIG. 24.

Cocktail:
0.14 mL 100 mM acetylcholine;
0.126 mL ATP Assay Diluent;
0.014 mL 1 mM ATP;
0.91 mL PBS;
0.014 mL ATP Assay Mix;
0.014 mL ATP Assay Mix;
0.014 mL 25 unit/mL acetate kinase (diluted in Acetate Kinase Buffer, 100 mM triethanolamine, pH 7.4).

Procedure: Tacrine or H₂O was distributed to wells of a luminance microtiter plate, as indicated in the chart below. Wells B7-B12 were duplicates of A7-A12, respectively.

The cocktail as above, with ACHE omitted, was distributed (0.087 mL) to A12 and B12. ACHE (0.096 mL at 25 units/mL, dissolved in 20 mM Tris, pH 7.4) was then added to the cocktail, and 0.095 mL of the cocktail was added to all other wells, A7-A11 and B7-B11. Measurement was then initiated. Table VIII indicates the levels of the tacrine inhibitor that were used. Concentrations listed are concentrations of the tacrine stock solutions (dissolved in H₂O) used. Volumes of the final reactions were 100 μL, except 92 μL in A12 and B12. For example, 0.005 mL of 0.6 μM tacrine used in wells A8 and B8 represents 3 picomoles of tacrine or 30 nM tacrine (final concentration).

TABLE VIII
Levels of Tacrine Inhibitor Used

| Well: | A7 | A8 | A9 | A10 | A11 | A12 |
|---|---|---|---|---|---|---|
| Tacrine | 0 | 0.6 μM | 2.0 μM | 6.0 μM | 20 μM | 0 |
| (all 0.005 | | | | | | |
| mL) | | | | | | |
| H₂O | 0.005 mL | 0 | 0 | 0 | 0 | 0.005 mL |
| Cocktail | +ACHE | +ACHE | +ACHE | +ACHE | +ACHE | −ACHE |

Wells B7-B12 were duplicates of A7-A12, respectively.

The results of this experiment are depicted Table IX. ACHE activity is very clearly distinguished from background, and inhibition by tacrine is detectable down to the lowest quantity used (˜3 picomoles).

TABLE IX
Detection of Acetylcholinesterase Activity and Tacrine Inhibition
by Coupled Luminescent Assay

| | Final Concentration of Tacrine | | | | | No |
|---|---|---|---|---|---|---|
| | 0 | 30 nM | 100 nM | 300 nM | 1 μM | Enzyme |
| Initiation | 14456 | 14975 | 15450 | 15397 | 14894 | 27215 |
| 57 sec | 8367 | 9501 | 10927 | 11985 | 12301 | 25911 |
| 128 sec | 4437 | 5103 | 7305 | 9189 | 10320 | 25121 |
| 376 sec | 805 | 1222 | 2287 | 4564 | 7062 | 23258 |

It will be evident to one skilled in the art that a similar reaction scheme in which a choline kinase is employed (as in FIG. 25) in place of the acetate kinase (FIG. 24) is also possible.

### Example 22

### Detection of the Nitrate Ion by Coupled Luminescence

The nitrate ion may be detected by using the enzyme nitrate reductase to couple the presence of nitrate to generation of NAD⁺, which is then detected as above in EXAMPLES 18, 19, and 20. In the given example, the source of nitrate chosen was a commercial fertilizer preparation, to demonstrate the ability of the method to quantify nitrate in a matrix containing various chemicals representative of potential environmental contaminants. The preparation chosen was Miracle-Gro Tomato Plant Food (hereafter TPF), whose analysis includes “2.6% nitrate nitrogen,” as well as urea, ammonia, and various other non-nitrogenous substances.

This example is an embodiment of the reaction series presented in FIG. 23.

Enhanced 4×LGP cocktail:
0.5 mL 5×PGK Diluent;
0.00625 mL 1M dithiothreitol;
0.0025 mL 100 mM NADH;
0.026 mL glyceraldehyde-3-phosphate (50 mg/mL);
0.09 mL H₂O.

The LT cocktail for the nitrate-detection reaction was made as in EXAMPLE 18, except that the new, higher-NADH 4×LGP cocktail described above was used in place of the 4×LGP cocktail described in EXAMPLE 18. The new LT cocktail was designated “LTC3.”

No separate enzyme cocktail was made; the enzymes were mixed with the LT cocktail as follows to make the reaction cocktail:
0.7 mL LTC3;
0.112 mL phosphoglycerokinase diluted 1:100 with G-diluent;
0.007 mL glyceraldehyde-3-phosphate dehydrogenase diluted 1:1000 with G-diluent;
0.021 mL nitrate reductase, 7 units/mL in 10 mM EDTA, 50 mM MOPS (pH 7.0), 50% glycerol, 1% BSA.

TPF was dissolved at 50 mg/mL in H₂O and filtered to remove particulates. This solution was further diluted 1:100, 1:1000, and 1:10,000 for testing.

TPF solutions and water controls were distributed as follows (all wells contained 0.005 mL):
Well F6: H₂O;
Well F7: 1:10,000 TPF;
Well F8: 1:1000 TPF;
Well F9: 1:100 TPF;
Wells G6-G9 and H6-H9 were replicates of wells F6-F9, respectively.

After the TPF was distributed, 0.06 mL of reaction cocktail was added to all twelve wells (F6-H9) and measurement was initiated. The results are depicted in Table X. There is some scatter, probably due to the nature of the substrate, but the method clearly allows detection of minute amounts of nitrate.

TABLE X
Detection of Nitrate by Coupled Luminescence

| Dilution of TPF | | |
|---|---|---|
| (Final) | RLU | Std. Dev. |
| 0.00E+00 | 6371.33 | 1225.22 |
| 7.69E−06 | 7783.00 | 2320.58 |
| 7.69E−05 | 7969.33 | 1653.87 |
| 7.69E−04 | 11735.00 | 1174.61 |

R² for RLU vs. TPF concentration is ˜0.932.

### Example 23

### Detection of Phosphate by Coupled Luminescence

As shown above, inorganic phosphate (hereafter, Pi) may be assayed by coupling its presence to light emission through the action of glyceraldehyde-3-phosphate dehydrogenase, phosphoglycerokinase, and luciferase, in the presence of appropriate substrates and buffer components. Other reaction schemes can also be used to generate ATP from Pi. For example, the following reaction is part of the tricarboxylic acid cycle of higher eukaryotes:
GDP+Pi+succinyl-CoA→succinate+GTP+CoA(free) (succinyl-CoA synthetase or SCoAS)

Production of GTP by this system is coupled to production of ATP, and subsequently to light emission by luciferase, by a commercially available nucleoside phosphotransferase enzyme such as nucleoside 5′-diphosphate kinase from baker's yeast (available from Sigma-Aldrich as catalog #N-0379). Alternatively, SCoAS from Trypanosome brucei may be employed; this enzyme generates ATP from ADP and Pi in the analogous step of the tricarboxylic acid cycle.

The existence of multiple reaction schemes of this nature demonstrates the general possibility of utilizing either substrate-level phosphorylation or electron-transport phosphorylation (the later characterized by its sensitivity to the cyanide ion) to consume Pi in a process that leads to ATP and subsequently to light emission by luciferase. The Pi may be transferred either directly to a nucleotide diphosphate to yield a nucleotide triphosphate, as in the above scheme, or to another molecule (such as glyceraldehyde-3-phosphate in the reaction scheme described in FIG. 1), for subsequent transfer to a nucleotide diphosphate by another enzyme (such as phosphoglycerokinase in FIG. 1). As a further refinement, it may be possible and desirable to adjust the reaction conditions such that all reactions necessary for the light emission take place simultaneously in a single reaction vessel.

### Example 24

### Detection of cAMP by Coupled Luminescence

Cyclic adenosine monophosphate (cAMP) may be detected by coupled luminescence technology in a number of ways. These methods are described in the schemes shown in FIGS. 26, 27, and 28.

In FIG. 26, cAMP is the test reagent cyclic AMP, PPi is pyrophosphoric acid, or a salt of pyrophosphate suitable for reaction with adenylate cyclase, ATP is adenosine triphosphate, adenylate cyclase is an enzyme capable of carrying out the indicated reaction, and hv is light. cAMP is omitted or provided in limiting quantity, PPi is supplied optionally in excess to drive the reaction in the direction of ATP synthesis, adenylate cyclase (or adenylyl cyclase) and luciferase are supplied enzymes, and other substrates, cofactors, and buffer components for the indicated enzymes are also supplied reagents.

In FIG. 27, the cAMP-dependent phosphodiesterase (PDE) is a supplied enzyme, PPi is pyrophosphoric acid, or a salt of pyrophosphate suitable for reaction with adenylate cyclase, ATP is adenosine triphosphate, adenylate cyclase is an enzyme capable of carrying out the indicated reaction, and hv is light. cAMP is omitted or provided in limiting quantity, PPi is supplied optionally in excess to drive the reaction in the direction of ATP synthesis, adenylate cyclase (or adenylyl cyclase) and luciferase are supplied enzymes, and other substrates, cofactors, and buffer components for the indicated enzymes are also supplied reagents. The first two reactions in the series may optionally be carried out simultaneously, optionally with a single initiation step, whereupon the reactions are transferred to the dark chamber of a luminometer or other suitable measuring device. Depriving the reactions of light will lead to breakdown of ATP by luciferase, with generation of light related to the initial cAMP. This reaction is distinguished from U.S. Pat. No. 5,891,659 (Murakami et al.) in the following ways: (1) In the scheme shown in FIG. 27, light is absolutely required, in place of the high-energy molecule exemplified by phosphoenol pyruvate in Muramaki et al. The Muramaki patent makes no mention of incubation of any reaction in light, and, indeed, incubation in light is unnecessary and could interfere with the Muramaki schemes. The present system employs light to avoid the difficult problem of obtaining and handling pyruvate orthophosphate dikinase or any similar ATP-regenerating enzyme. (2) In Murakami et al. it is clear that the possibility of using luciferase as the ATP-regenerating enzyme, as well as the light-producing enzyme, is not envisioned. Indeed, the backward reaction of luciferase, in which ATP is formed from AMP and pyrophosphate in the presence of light, is not mentioned at all in Murakami et al. (3) The term “regenerating enzyme” implies that what is envisioned in Murakami et al. is a repetitive or cyclic synthesis of ATP. This is neither possible nor necessary in the scheme shown in FIG. 27, in which ATP is generated once in a manner which depends on the amount of AMP present, and the ATP is then quantified by luminance measurements in a dark chamber, where no significant degree of regeneration is possible in our system.

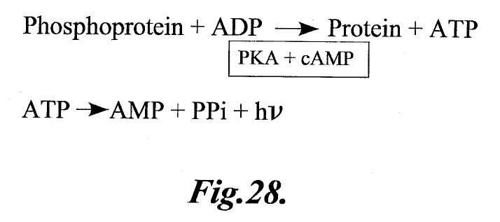

In the scheme shown in FIG. 28, the phosphoprotein is any suitable phosphorylated protein substrate for the reverse kinase reaction, such as purified alpha-casein; ADP is adenosine diphosphate; the Protein is the partially or completely dephosphorylated product of the reverse kinase reaction; ATP is adenoside triphosphate; PKA is protein kinase A, or another cAMP-dependent protein kinase; cAMP is cyclic adenosine monophosphate; AMP is adenosine monophosphate; PPi is pyrophosphate; and hv is light. In this scheme, cAMP acts as an activator of PKA and is not consumed in the course of the reaction. cAMP is the test reagent; PKA and luciferase are supplied enzymes; ADP, phosphoprotein, and other substrates, cofactors, and buffer components for the indicated enzymes are supplied reagents; and Protein, AMP, and PPi are byproducts that play no role. cAMP may be supplied by a separate enzymatic reaction or series of enzymatic reactions.

All publications and patent applications mentioned in this specification are herein incorporated by reference to the same extent as if each individual publication or patent application was specifically and individually incorporated by reference.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for measuring the amount of a kinase substrate in a sample, comprising the steps of:
(a) contacting a sample comprising a substrate for an ATP-dependent kinase with (i) an ATP-dependent kinase that phosphorylates the substrate in the presence of ATP and (ii) an ATP-dependent luciferase, under conditions wherein luminance emitted from the sample depends on the initial amount of the substrate in the sample; and
(b) measuring the amount of the substrate in the sample by measuring the emitted luminance.

2. The method of claim 1, where the substrate is acetate and the kinase is acetate kinase or the substrate is choline and the kinase is choline kinase.

3. A method for measuring the activity of acetylcholinesterase in a sample, comprising the step of measuring the activity of acetylcholinesterase in a sample by measuring the amount of acetate or choline in the sample using the method of claim 2, wherein the acetate or choline in the sample is produced in an acetylcholinesterase-catalyzed reaction with acetylcholine.

4. A method for measuring the amount of an inhibitor of acetylcholinesterase in a sample, comprising the steps of:
(a) contacting the sample with an acetylcholinesterase under suitable conditions for producing acetate and choline from acetylcholine; and
(b) measuring the amount of one or more inhibitors of acetylcholinesterase in the sample by measuring the activity of the acetylcholinesterase using the method of claim 3.
